(19) United States
(12) Patent Application Publication
(10) Pub. No.: US 2024/0415012 A1
(43) Pub. Date: Dec. 12, 2024

(54) PHOTOELECTRIC CONVERSION DEVICE MATERIAL AND DISPLAY DEVICE

(71) Applicant: SEMICONDUCTOR ENERGY LABORATORY CO., LTD., Atsugi-shi, Kanagawa-ken (JP)

(72) Inventors: Daisuke KUBOTA, Atsugi, Kanagawa (JP); Kazuya SUGIMOTO, Hadano, Kanagawa (JP); Akio YAMASHITA, Atsugi, Kanagawa (JP); Yasuhiro NIIKURA, Komae, Tokyo (JP); Sachiko KAWAKAMI, Atsugi, Kanagawa (JP); Anna TADA, Ishara, Kanagawa (JP)

(21) Appl. No.: 18/690,357

(22) PCT Filed: Sep. 2, 2022
(86) PCT No.: PCT/IB2022/058240

§ 371 Date: Mar. 8, 2024

(30) Foreign Application Priority Data
Sep. 17, 2021 (JP) 2021-152526

(51) Int. Cl. H10K 85/60; C07C 211/61; H10K 39/00

(52) U.S. Cl. CPC H10K 85/633; C07C 211/61; H10K 39/601; H10K 85/615

(57) ABSTRACT

A novel and highly convenient, useful, or reliable material for a photoelectric conversion device is provided. The photoelectric conversion device includes a first electrode, a second electrode, a first layer, a second layer, and a third layer. The first layer is held between the first electrode and the second electrode; the second layer is held between the second electrode and the first layer; the third layer is held between the second electrode and the second layer; and the third layer has higher electron mobility than the first layer. The material is used in the second layer, the material contains an anthracene skeleton, and the anthracene skeleton is bonded to a diarylamino group, a diheteroarylamino group, or an arylheteroarylamino group.

### TECHNICAL FIELD

One embodiment of the present invention relates to a photoelectric conversion device material, a display device, or a semiconductor device.

Note that one embodiment of the present invention is not limited to the above technical field. The technical field of one embodiment of the invention disclosed in this specification and the like relates to an object, a method, or a manufacturing method. Alternatively, one embodiment of the present invention relates to a process, a machine, manufacture, or a composition of matter. Thus, more specifically, examples of the technical field of one embodiment of the present invention disclosed in this specification include a semiconductor device, a display device, a light-emitting apparatus, a power storage device, a memory device, a driving method thereof, and a manufacturing method thereof.

### BACKGROUND ART

A solar cell including, as a material for a solar cell, an anthracene derivative which includes a diarylamino group containing a substituted or unsubstituted aryl group is known (Patent Document 1).

### REFERENCE

### Patent Document

[Patent Document 1] Japanese Patent No. 5243891

### SUMMARY OF THE INVENTION

### Problems to be Solved by the Invention

An object of one embodiment of the present invention is to provide a novel photoelectric conversion device material that is highly convenient, useful, or reliable. Another object is to provide a novel display device that is highly convenient, useful, or reliable. Another object is to provide a novel photoelectric conversion device material, a novel display device, or a novel semiconductor device.

Note that the description of these objects does not preclude the existence of other objects. One embodiment of the present invention does not have to achieve all of these objects. Other objects will be apparent from the description of the specification, the drawings, the claims, and the like, and other objects can be derived from the description of the specification, the drawings, the claims, and the like.

### Means for Solving the Problems

(1) One embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; and the material is used in the second layer.

The material for the photoelectric conversion device includes an anthracene skeleton, and the anthracene skeleton is bonded to a diarylamino group, a diheteroarylamino group, or an arylheteroarylamino group.

(2) Another embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; and the material is used in the second layer.

The material for the photoelectric conversion device includes an anthracene skeleton, and the 9-position of the anthracene skeleton is bonded to a diarylamino group, a diheteroarylamino group, or an arylheteroarylamino group.

(3) Another embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; and the material is used in the second layer.

The material for the photoelectric conversion device includes an anthracene skeleton, and the 9-position of the anthracene skeleton is bonded to a substituted or unsubstituted diarylamino group; both the 2-position and the 6-position of the anthracene skeleton or either the 2-position or the 6-position of the anthracene skeleton is bonded to any of a substituted or unsubstituted aryl group having 6 to 25 carbon atoms, a substituted or unsubstituted heteroaryl group having 2 to 25 carbon atoms, and a substituted or unsubstituted diarylamino group.

Note that an aryl group of the substituted or unsubstituted diarylamino group is a substituted or unsubstituted aryl group having 6 to 25 carbon atoms or a substituted or unsubstituted heteroaryl group having 2 to 25 carbon atoms.

When the diarylamino group includes two aryl groups, the two aryl groups may be the same or different from each other and may be bonded to each other to form a ring.

When the diarylamino group includes two heteroaryl groups, the two heteroaryl groups may be the same or different from each other and may be bonded to each other to form a ring.

When the diarylamino group includes an aryl group and a heteroaryl group, the aryl group and the heteroaryl group may be bonded to each other to form a ring.

(4) Another embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; and the material is used in the second layer.

The material for the photoelectric conversion device includes an anthracene skeleton; each of the 9-position and the 10-position of the anthracene skeleton is bonded to a substituted or unsubstituted diarylamino group; and both the 2-position and the 6-position of the anthracene skeleton or either the 2-position or the 6-position of the anthracene skeleton is bonded to any of a substituted or unsubstituted aryl group having 6 to 25 carbon atoms, a substituted or unsubstituted heteroaryl group having 2 to 25 carbon atoms, and a substituted or unsubstituted diarylamino group.

Note that an aryl group of the substituted or unsubstituted diarylamino group is a substituted or unsubstituted aryl group having 6 to 25 carbon atoms or a substituted or unsubstituted heteroaryl group having 2 to 25 carbon atoms.

When the diarylamino group includes two aryl groups, the two aryl groups may be the same or different from each other and may be bonded to each other to form a ring.

When the diarylamino group includes two heteroaryl groups, the two heteroaryl groups may be the same or different from each other and may be bonded to each other to form a ring.

When the diarylamino group includes an aryl group and a heteroaryl group, the aryl group and the heteroaryl group may be bonded to each other to form a ring.

(5) Another embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; the material is used in the second layer; and the material is represented by General Formula (G1) below.

### [Chemical Formula 1]

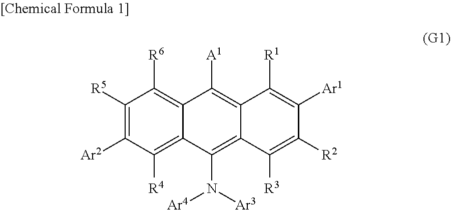

Note that in General Formula (G1) above, each of A¹, Ar¹, and Ar² independently represents hydrogen, deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, or a substituted or unsubstituted diarylamino group.

Note that an aryl group of the substituted or unsubstituted diarylamino group is a substituted or unsubstituted aryl group having 6 to 25 carbon atoms or a substituted or unsubstituted heteroaryl group having 2 to 25 carbon atoms.

When the diarylamino group includes two aryl groups, the two aryl groups may be the same or different from each other and may be bonded to each other to form a ring.

When the diarylamino group includes two heteroaryl groups, the two heteroaryl groups may be the same or different from each other and may be bonded to each other to form a ring.

When the diarylamino group includes an aryl group and a heteroaryl group, the aryl group and the heteroaryl group may be bonded to each other to form a ring.

Each of R¹ to R⁶ independently represents hydrogen, deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, or a substituted or unsubstituted alkoxy group having 1 to 13 carbon atoms.

Each of Ar³ to Ar⁴ independently represents a substituted or unsubstituted aryl group having 6 to 25 carbon atoms or a substituted or unsubstituted heteroaryl group having 2 to 25 carbon atoms.

Any hydrogen in General Formula (G1) above may be deuterium.

Accordingly, a photoelectric conversion device material having sensitivity to light in a wide wavelength range including the visible light range can be provided. A photoelectric conversion device having sensitivity to light in a wide wavelength range including the visible light range can be provided. An operation voltage of a photoelectric conversion device can be reduced. A photoelectric conversion device capable of operating at low voltage can be provided. The solubility of a photoelectric conversion device material can be increased. A photoelectric conversion device material whose purity is easy to examine can be provided. A photoelectric conversion device material that is easy to purify and highly purify can be provided. A highly reliable photoelectric conversion device can be provided using a material having high purity. An organic compound represented by General Formula (G1) above can be synthesized by a variety of methods. A structure of the organic compound is less restricted by its synthesis method, enabling high flexibility of molecular design. The organic compound represented by General Formula (G1) above has a shallow HOMO level derived from the amine skeleton and an excellent carrier-transport property derived from the anthracene skeleton, making it possible to provide a photoelectric conversion device with high efficiency. Consequently, a novel photoelectron conversion device material that is highly convenient, useful, or reliable can be provided.

(6) Another embodiment of the present invention is a display device including a pixel set.

The pixel set includes a first pixel and a second pixel; the first pixel includes a light-emitting device; the second pixel includes a photoelectric conversion device; and the photoelectric conversion device is adjacent to the light-emitting device.

The photoelectric conversion device includes any one of the above-described materials for the photoelectric conversion device.

(7) Another embodiment of the present invention is the above display device including a first functional layer and a second functional layer.

The first pixel includes a first pixel circuit, and the first pixel circuit is electrically connected to the light-emitting device.

The second pixel includes a second pixel circuit, and the second pixel circuit is electrically connected to the photoelectric conversion device.

The first functional layer overlaps with the second functional layer, and the first functional layer includes the photoelectric conversion device and the light-emitting device.

The second functional layer includes the first pixel circuit and the second pixel circuit.

(8) Another embodiment of the present invention is the above display device in which the above light-emitting device includes a third electrode, a fourth electrode, and a unit.

The unit is held between the third electrode and the fourth electrode, and the unit includes the first layer, the third layer, and a fourth layer.

The first layer is held between the third electrode and the fourth electrode, the third layer is held between the fourth electrode and the first layer, and the third layer has higher electron mobility than the first layer.

The fourth layer is held between the third layer and the first layer, and the fourth layer includes a light-emitting material.

Thus, light can be emitted. In addition, an image can be displayed. Moreover, irradiation light can be converted into current. Furthermore, imaging can be performed. As a result, a novel display device that is highly convenient, useful, or reliable can be provided.

Although a block diagram in which components are classified by their functions and shown as independent blocks is shown in the drawing attached to this specification, it is difficult to completely separate actual components according to their functions and one component can relate to a plurality of functions.

Note that the light-emitting apparatus in this specification includes, in its category, an image display device that uses a light-emitting device. The light-emitting apparatus may also include a module in which a light-emitting device is provided with a connector such as an anisotropic conductive film or a TCP (Tape Carrier Package), a module in which a printed wiring board is provided at the end of a TCP, and a module in which an integrated circuit (IC) is directly mounted on a light-emitting device by a COG (Chip On Glass) method. Furthermore, a lighting device or the like may include the light-emitting apparatus.

### Effect of the Invention

According to one embodiment of the present invention, a novel photoelectric conversion device material that is highly convenient, useful, or reliable can be provided. Another embodiment of the present invention can provide a novel display device that is highly convenient, useful, or reliable. A novel photoelectric conversion device material can be provided. A novel display device can be provided.

Note that the description of these effects does not preclude the existence of other effects. One embodiment of the present invention does not need to have all of these effects. Other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A and FIG. 1B are diagrams illustrating structures of a photoelectric conversion device using a photoelectric conversion device material of one embodiment of the present invention.

FIG. 2A and FIG. 2B are diagrams illustrating structures of a display device of an embodiment.

FIG. 3A to FIG. 3C are diagrams illustrating a structure of a device of an embodiment.

FIG. 4 is a diagram illustrating a structure of a device of an embodiment.

FIG. 5 is a circuit diagram illustrating a structure of a device of an embodiment.

FIG. 6 is a circuit diagram illustrating a structure of a device of an embodiment.

FIG. 7A and FIG. 7B are circuit diagrams illustrating structures of a device of an embodiment.

FIG. 8A illustrates a cross-sectional perspective view illustrating a structure of an imaging device of an embodiment. FIG. 8B is a circuit diagram illustrating the structure of the imaging device of the embodiment.

FIG. 9 is a cross-sectional view illustrating a structure of a pixel of an embodiment.

FIG. 10A to FIG. 10F are perspective views illustrating structures of a package and a module including an imaging device of an embodiment.

FIG. 11A to FIG. 11F are diagrams illustrating structures of electronic devices of an embodiment.

FIG. 12A and FIG. 12B are cross-sectional views illustrating structures of a photoelectric conversion device of examples.

FIG. 13 is a graph showing spectral sensitivity of devices of an example.

FIG. 14 is a graph showing voltage-current density characteristics of devices in a state where light irradiation is performed in an example.

FIG. 15 is a graph showing voltage-current density characteristics of devices in a state where light irradiation is not performed in an example.

FIG. 16 is a graph showing spectral sensitivity of devices of an example.

FIG. 17 is a graph showing voltage-current density characteristics of devices in a state where light irradiation is performed in an example.

FIG. 18 is a graph showing voltage-current density characteristics of devices in a state where light irradiation is not performed in an example.

### MODE FOR CARRYING OUT THE INVENTION

One embodiment of the present invention is a material for a photoelectric conversion device which includes a first electrode, a second electrode, a first layer, a second layer, and a third layer. The first layer is held between the first electrode and the second electrode; the second layer is held between the second electrode and the first layer; the third layer is held between the second electrode and the second layer; the third layer has higher electron mobility than the first layer; and the material is used in the second layer. The material for the photoelectric conversion device includes an anthracene skeleton, and the anthracene skeleton is bonded to a diarylamino group, a diheteroarylamino group, or an arylheteroarylamino group.

Accordingly, a photoelectric conversion device material having sensitivity to light in a wide wavelength range including the visible light range can be provided. A photoelectric conversion device having sensitivity to light in a wide wavelength range including the visible light range can be provided. An operation voltage of a photoelectric conversion device can be reduced. A photoelectric conversion device capable of operating at low voltage can be provided. The solubility of a photoelectric conversion device material can be increased. A photoelectric conversion device material whose purity is easy to examine can be provided. A photoelectric conversion device material that is easy to purify and highly purify can be provided. A highly reliable photoelectric conversion device can be provided using a material having high purity. The photoelectric conversion device material of one embodiment of the present invention can be synthesized by a variety of methods. A structure of the organic compound is less restricted by its synthesis method, enabling high flexibility of molecular design. A photoelectric conversion device material of one embodiment of the present invention can provide a photoelectric conversion device with high efficiency. Consequently, a novel photoelectron conversion device material that is highly convenient, useful, or reliable can be provided.

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments. Note that in structures of the invention described below, the same portions or portions having similar functions are denoted by the same reference numerals in different drawings, and the description thereof is not repeated.

### Embodiment 1

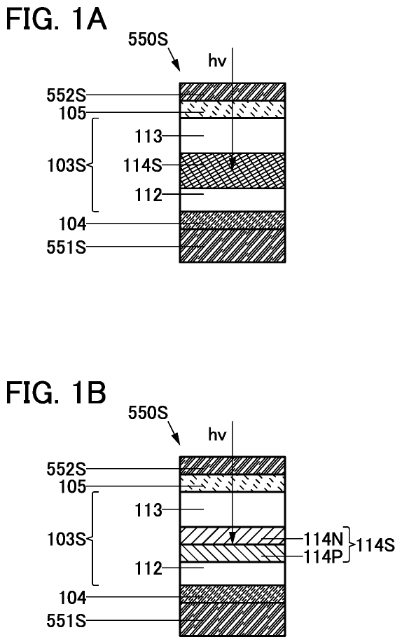

In this embodiment, a photoelectron conversion device material of one embodiment of the present invention will be described with reference to FIG. 1A and FIG. 1B.

FIG. 1A is a cross-sectional view illustrating a structure of a photoelectric conversion device using a material of one embodiment of the present invention, and FIG. 1B is a cross-sectional view illustrating a structure of a photoelectric conversion device different from that in FIG. 1A.

### <Example 1 of Photoelectric Conversion Device Material>

A photoelectric conversion device material described in this embodiment is an anthracene derivative. The anthracene derivative has an anthracene skeleton, and the anthracene skeleton is bonded to a diarylamino group, a diheteroarylamino group, or an arylheteroarylamino group.

Note that the photoelectric conversion device material described in this embodiment can be used for a layer 114S included in a photoelectric conversion device 550S (see FIG. 1A).

The photoelectric conversion device 550S includes an electrode 551S, an electrode 552S, a layer 112, the layer 114S, and a layer 113.

### <<Layer 112>>

The layer 112 is held between the electrode 551S and the electrode 552S.

### <<Layer 114S>>

The layer 114S is held between the electrode 552S and the layer 112 and includes the above-described anthracene derivative.

### <<Layer 113>>

The layer 113 is held between the electrode 552S and the layer 114S. In addition, the layer 113 has higher electron mobility than the layer 112.

### <Example 2 of Photoelectric Conversion Device Material>

A photoelectric conversion device material described in this embodiment is an anthracene derivative. The anthracene derivative includes an anthracene skeleton, and the 9-position of the anthracene skeleton is bonded to a diarylamino group, a diheteroarylamino group, or an arylheteroarylamino group.

Note that the photoelectric conversion device material described in this embodiment can be used for the layer 114S of the photoelectric conversion device 550S (see FIG. 1A).

### <Example 3 of Photoelectric Conversion Device Material>

A photoelectric conversion device material described in this embodiment is an anthracene derivative. The anthracene derivative has an anthracene skeleton, and the 9-position of the anthracene skeleton is bonded to a substituted or unsubstituted diarylamino group. Furthermore, both the 2-position and the 6-position of the anthracene skeleton or either the 2-position or the 6-position of the anthracene skeleton is bonded to any of a substituted or unsubstituted aryl group having 6 to 25 carbon atoms, a substituted or unsubstituted heteroaryl group having 2 to 25 carbon atoms, and a substituted or unsubstituted diarylamino group.

Note that an aryl group in the above-described substituted or unsubstituted diarylamino group, which substitutes for hydrogen atoms at the 2-position, the 6-position, and the 9-position of the anthracene skeleton, is a substituted or unsubstituted aryl group having 6 to 25 carbon atoms or a substituted or unsubstituted heteroaryl group having 2 to 25 carbon atoms.

When the diarylamino group includes two aryl groups, the two aryl groups may be the same or different from each other and may be bonded to each other to form a ring.

When the diarylamino group includes two heteroaryl groups, the two heteroaryl groups may be the same or different from each other and may be bonded to each other to form a ring.

When the diarylamino group includes an aryl group and a heteroaryl group, the aryl group and the heteroaryl group may be bonded to each other to form a ring.

### <Example 4 of Photoelectric Conversion Device Material>

A photoelectric conversion device material described in this embodiment is an anthracene derivative. The anthracene derivative has an anthracene skeleton, and the 9-position and the 10-position of the anthracene skeleton is bonded to a substituted or unsubstituted diarylamino group. Furthermore, both the 2-position and the 6-position of the anthracene skeleton or either the 2-position or the 6-position of the anthracene skeleton is bonded to any of a substituted or unsubstituted aryl group having 6 to 25 carbon atoms, a substituted or unsubstituted heteroaryl group having 2 to 25 carbon atoms, and a substituted or unsubstituted diarylamino group.

Note that an aryl group of the substituted or unsubstituted diarylamino group is a substituted or unsubstituted aryl group having 6 to 25 carbon atoms or a substituted or unsubstituted heteroaryl group having 2 to 25 carbon atoms.

When the diarylamino group includes two aryl groups, the two aryl groups may be the same or different from each other and may be bonded to each other to form a ring.

When the diarylamino group includes two heteroaryl groups, the two heteroaryl groups may be the same or different from each other and may be bonded to each other to form a ring.

When the diarylamino group includes an aryl group and a heteroaryl group, the aryl group and the heteroaryl group may be bonded to each other to form a ring.

### <Example 5 of Photoelectric Conversion Device Material>

A photoelectric conversion device material described in this embodiment is an anthracene derivative represented by General Formula (G1).

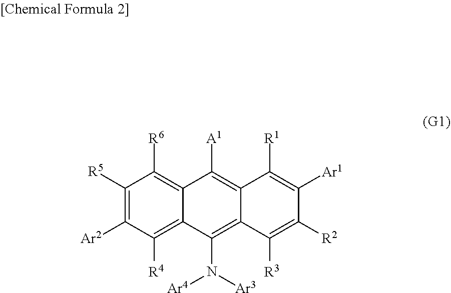

Note that in General Formula (G1) above, each of A¹, Ar¹, and Ar² independently represents hydrogen, deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, or a substituted or unsubstituted diarylamino group.

An aryl group of the substituted or unsubstituted diarylamino group is a substituted or unsubstituted aryl group having 6 to 25 carbon atoms or a substituted or unsubstituted heteroaryl group having 2 to 25 carbon atoms.

Note that when the diarylamino group includes two aryl groups, the two aryl groups may be the same or different from each other and may be bonded to each other to form a ring.

When the diarylamino group includes two heteroaryl groups, the two heteroaryl groups may be the same or different from each other to form a ring.

When the diarylamino group includes an aryl group and a heteroaryl group, the aryl group and the heteroaryl group may be bonded to each other to form a ring.

Each of R¹ to R⁶ independently represents hydrogen, deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, or a substituted or unsubstituted alkoxy group having 1 to 13 carbon atoms.

Each of Ar³ to Ar⁴ independently represents a substituted or unsubstituted aryl group having 6 to 25 carbon atoms or a substituted or unsubstituted heteroaryl group having 2 to 25 carbon atoms.

Note that any hydrogen in General Formula (G1) may be deuterium.

Accordingly, a photoelectric conversion device material having sensitivity to light in a wide wavelength range including the visible light range can be provided. A photoelectric conversion device having sensitivity to light in a wide wavelength range including the visible light range can be provided. An operation voltage of a photoelectric conversion device can be reduced. A photoelectric conversion device capable of operating at low voltage can be provided. The solubility of a photoelectric conversion device material can be increased. A photoelectric conversion device material whose purity is easy to examine can be provided. A photoelectric conversion device material that is easy to purify and highly purify can be provided. A highly reliable photoelectric conversion device can be provided using a material having high purity. An organic compound represented by General Formula (G1) above can be synthesized by a variety of methods. A structure of the organic compound is less restricted by its synthesis method, enabling high flexibility of molecular design. The organic compound represented by General Formula (G1) above has a shallow HOMO level derived from the amine skeleton and an excellent carrier-transport property derived from the anthracene skeleton, making it possible to provide a photoelectric conversion device with high efficiency. Consequently, a novel photoelectron conversion device material that is highly convenient, useful, or reliable can be provided.

Examples of substituents of the diarylamino group, the aryl group, or the heteroaryl group described above include an alkyl group, a substituted or unsubstituted cycloalkyl group, a substituted or unsubstituted aromatic hydrocarbon group, and a substituted or unsubstituted heteroaromatic hydrocarbon group.

Note that as the alkyl group, an alkyl group having 1 to 4 carbon atoms can be used. For example, a methyl group, an ethyl group, a propyl group, an isopropyl group, a butyl group, an isobutyl group, a tert-butyl group, an n-hexyl group, or the like can be used.

As the cycloalkyl group, a cycloalkyl group having 3 to 10 carbon atoms can be used. For example, a cyclopropyl group, a cyclobutyl group, a cyclopentyl group, a cyclohexyl group, an adamantyl group, or the like can be used.

As the aromatic hydrocarbon group, an aromatic hydrocarbon group having 6 to 30 carbon atoms can be used. For example, a phenyl group, a naphthyl group, a biphenyl group, a fluorenyl group, a spirofluorenyl group, or the like can be used.

As the heteroaromatic hydrocarbon group, a heteroaromatic hydrocarbon group having 2 to 30 carbon atoms can be used. For example, a pyridine ring, a pyrimidine ring, a pyrazine ring, or a pyridazine ring, a triazine ring, a quinoline ring, a quinoxaline ring, a quinazoline ring, a benzoquinazoline ring, a phenanthroline ring, an azafluoranthene ring, an imidazole ring, an oxazole ring, an oxadiazole ring, a triazole ring, or the like can be used.

### [Specific Example of Photoelectric Conversion Device Material]

Specific examples of the photoelectric conversion device material having the above structure are shown below.

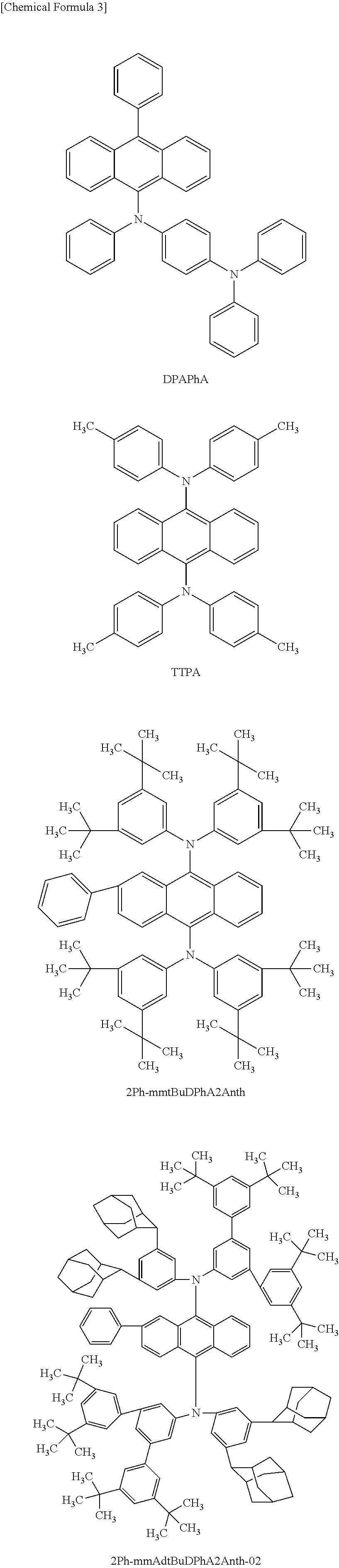

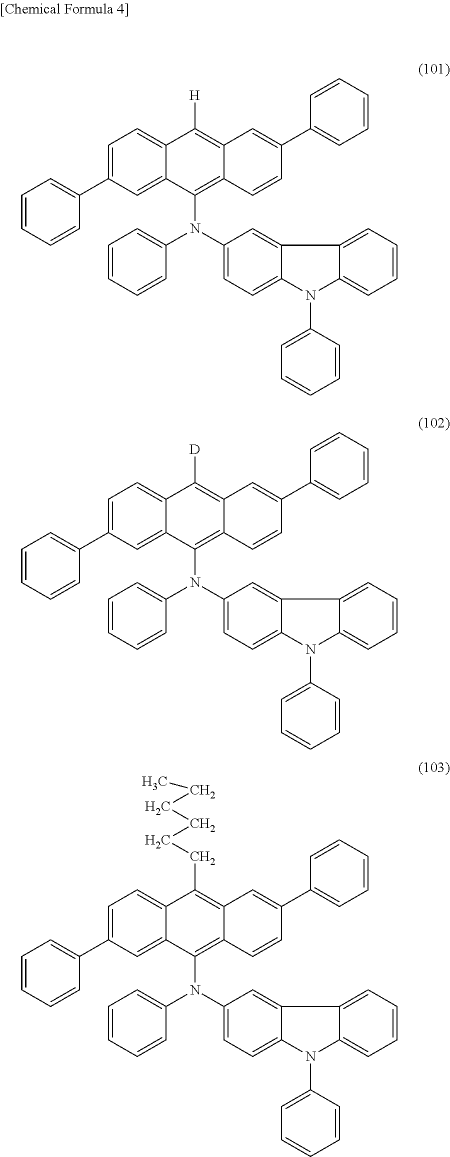 [SMILES: c1ccc(-c2ccc3c(N(c4ccccc4)c4ccc5c(c4)c4ccccc4n5-c4ccccc4)c4cc(-c5ccccc5)ccc4cc3c2)cc1 ; [2H]c1c2cc(-c3ccccc3)ccc2c(N(c2ccccc2)c2ccc3c(c2)c2ccccc2n3-c2ccccc2)c2cc(-c3ccccc3)ccc12 ; CCCCCCc1c2cc(-c3ccccc3)ccc2c(N(c2ccccc2)c2ccc3c(c2)c2ccccc2n3-c2ccccc2)c2cc(-c3ccccc3)ccc12]

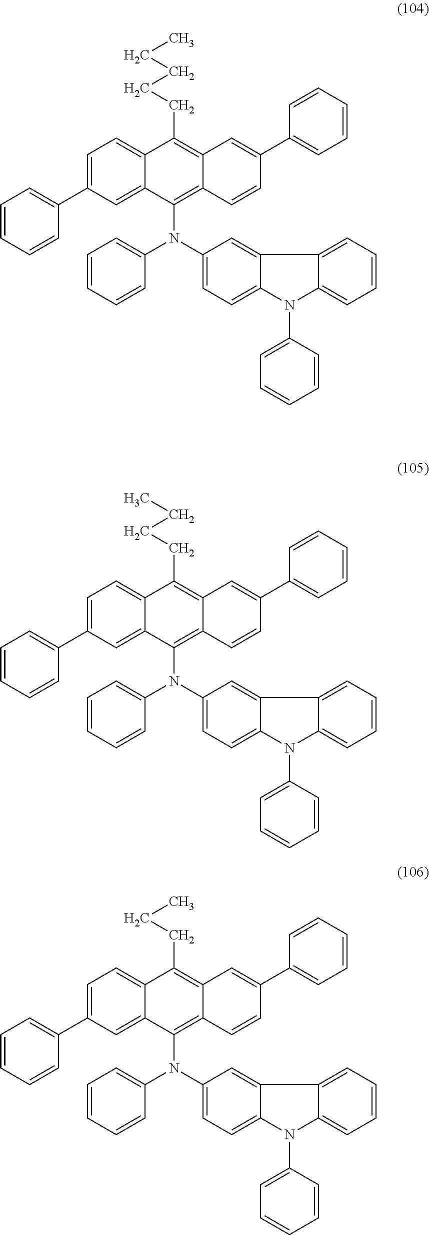 [SMILES: CCCCCc1c2cc(-c3ccccc3)ccc2c(N(c2ccccc2)c2ccc3c(c2)c2ccccc2n3-c2ccccc2)c2cc(-c3ccccc3)ccc12 ; CCCCc1c2cc(-c3ccccc3)ccc2c(N(c2ccccc2)c2ccc3c(c2)c2ccccc2n3-c2ccccc2)c2cc(-c3ccccc3)ccc12 ; CCCc1c2cc(-c3ccccc3)ccc2c(N(c2ccccc2)c2ccc3c(c2)c2ccccc2n3-c2ccccc2)c2cc(-c3ccccc3)ccc12]

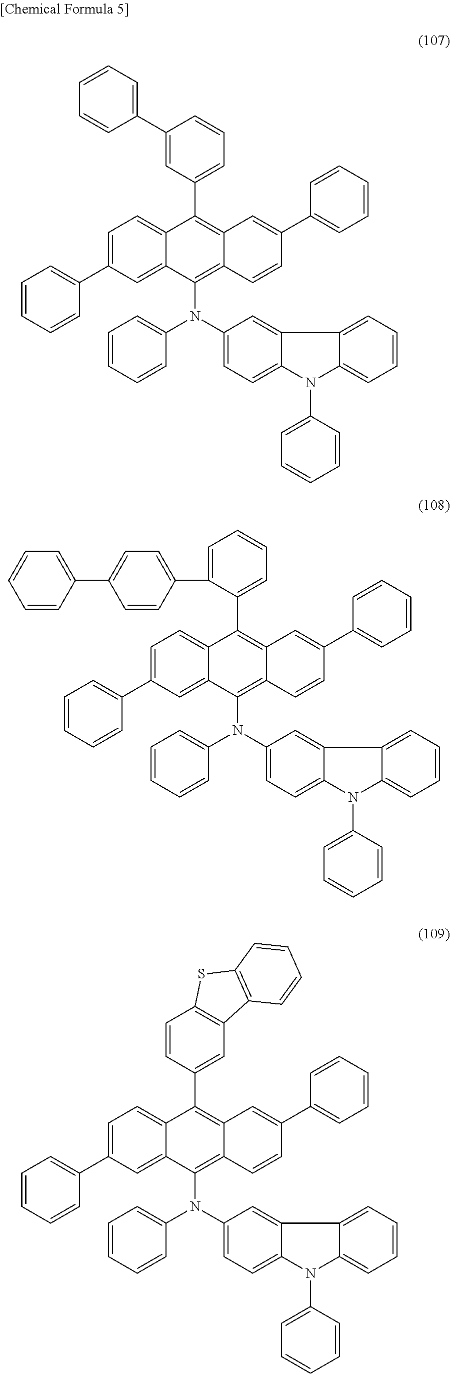 [SMILES: c1ccc(-c2cccc(-c3c4cc(-c5ccccc5)ccc4c(N(c4ccccc4)c4ccc5c(c4)c4ccccc4n5-c4ccccc4)c4cc(-c5ccccc5)ccc34)c2)cc1 ; c1ccc(-c2ccc(-c3ccccc3-c3c4cc(-c5ccccc5)ccc4c(N(c4ccccc4)c4ccc5c(c4)c4ccccc4n5-c4ccccc4)c4cc(-c5ccccc5)ccc34)cc2)cc1 ; c1ccc(-c2ccc3c(N(c4ccccc4)c4ccc5c(c4)c4ccccc4n5-c4ccccc4)c4cc(-c5ccccc5)ccc4c(-c4ccc5sc6ccccc6c5c4)c3c2)cc1]

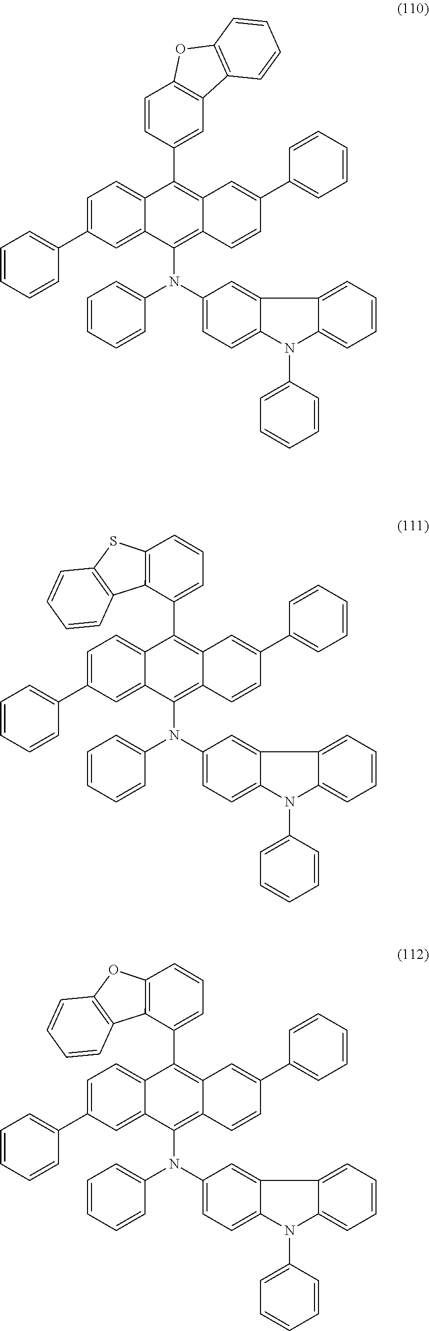 [SMILES: c1ccc(-c2ccc3c(N(c4ccccc4)c4ccc5c(c4)c4ccccc4n5-c4ccccc4)c4cc(-c5ccccc5)ccc4c(-c4ccc5oc6ccccc6c5c4)c3c2)cc1 ; c1ccc(-c2ccc3c(N(c4ccccc4)c4ccc5c(c4)c4ccccc4n5-c4ccccc4)c4cc(-c5ccccc5)ccc4c(-c4cccc5sc6ccccc6c45)c3c2)cc1 ; c1ccc(-c2ccc3c(N(c4ccccc4)c4ccc5c(c4)c4ccccc4n5-c4ccccc4)c4cc(-c5ccccc5)ccc4c(-c4cccc5oc6ccccc6c45)c3c2)cc1]

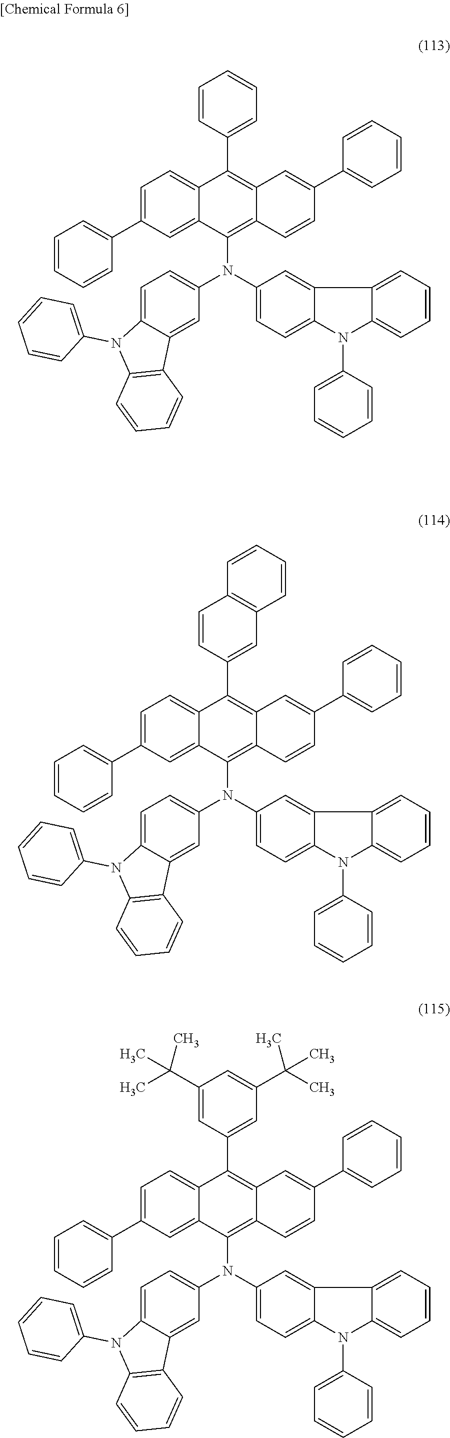 [SMILES: c1ccc(-c2ccc3c(N(c4ccc5c(c4)c4ccccc4n5-c4ccccc4)c4ccc5c(c4)c4ccccc4n5-c4ccccc4)c4cc(-c5ccccc5)ccc4c(-c4ccccc4)c3c2)cc1 ; c1ccc(-c2ccc3c(N(c4ccc5c(c4)c4ccccc4n5-c4ccccc4)c4ccc5c(c4)c4ccccc4n5-c4ccccc4)c4cc(-c5ccccc5)ccc4c(-c4ccc5ccccc5c4)c3c2)cc1 ; CC(C)(C)c1cc(-c2c3cc(-c4ccccc4)ccc3c(N(c3ccc4c(c3)c3ccccc3n4-c3ccccc3)c3ccc4c(c3)c3ccccc3n4-c3ccccc3)c3cc(-c4ccccc4)ccc23)cc(C(C)(C)C)c1]

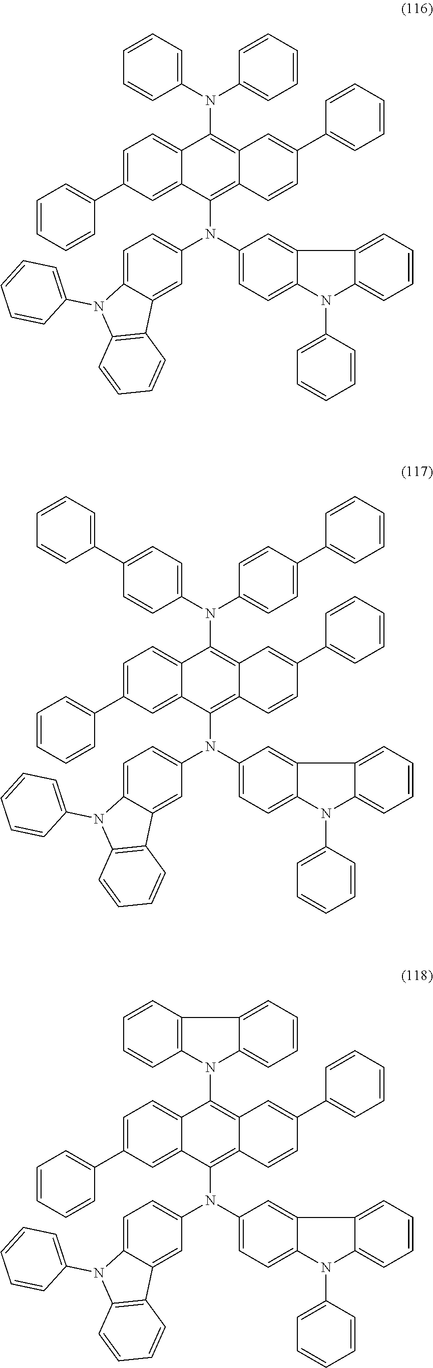 [SMILES: c1ccc(-c2ccc3c(N(c4ccc5c(c4)c4ccccc4n5-c4ccccc4)c4ccc5c(c4)c4ccccc4n5-c4ccccc4)c4cc(-c5ccccc5)ccc4c(N(c4ccccc4)c4ccccc4)c3c2)cc1 ; c1ccc(-c2ccc(N(c3ccc(-c4ccccc4)cc3)c3c4ccc(-c5ccccc5)cc4c(N(c4ccc5c(c4)c4ccccc4n5-c4ccccc4)c4ccc5c(c4)c4ccccc4n5-c4ccccc4)c4ccc(-c5ccccc5)cc34)cc2)cc1 ; c1ccc(-c2ccc3c(-n4c5ccccc5c5ccccc54)c4cc(-c5ccccc5)ccc4c(N(c4ccc5c(c4)c4ccccc4n5-c4ccccc4)c4ccc5c(c4)c4ccccc4n5-c4ccccc4)c3c2)cc1]

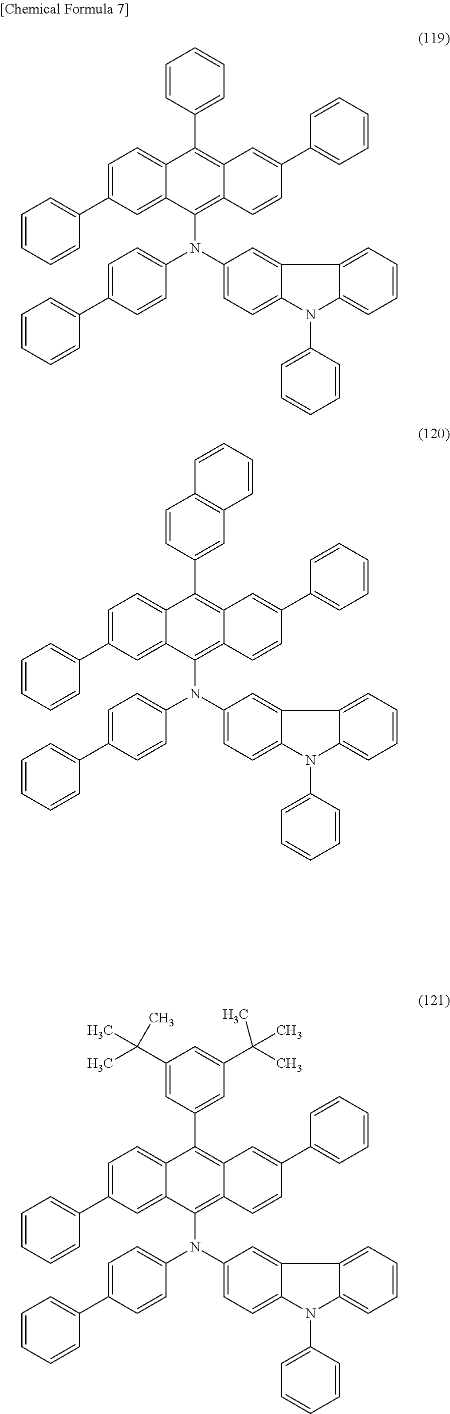 [SMILES: c1ccc(-c2ccc(N(c3ccc4c(c3)c3ccccc3n4-c3ccccc3)c3c4ccc(-c5ccccc5)cc4c(-c4ccccc4)c4ccc(-c5ccccc5)cc34)cc2)cc1 ; c1ccc(-c2ccc(N(c3ccc4c(c3)c3ccccc3n4-c3ccccc3)c3c4ccc(-c5ccccc5)cc4c(-c4ccc5ccccc5c4)c4ccc(-c5ccccc5)cc34)cc2)cc1 ; CC(C)(C)c1cc(-c2c3cc(-c4ccccc4)ccc3c(N(c3ccc(-c4ccccc4)cc3)c3ccc4c(c3)c3ccccc3n4-c3ccccc3)c3cc(-c4ccccc4)ccc23)cc(C(C)(C)C)c1]

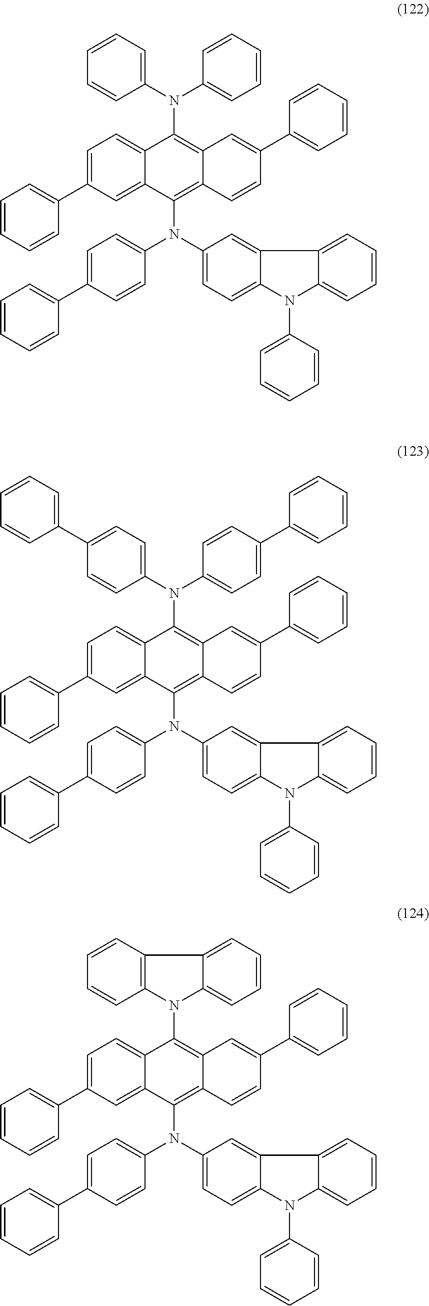 [SMILES: c1ccc(-c2ccc(N(c3ccc4c(c3)c3ccccc3n4-c3ccccc3)c3c4ccc(-c5ccccc5)cc4c(N(c4ccccc4)c4ccccc4)c4ccc(-c5ccccc5)cc34)cc2)cc1 ; c1ccc(-c2ccc(N(c3ccc(-c4ccccc4)cc3)c3c4ccc(-c5ccccc5)cc4c(N(c4ccc(-c5ccccc5)cc4)c4ccc5c(c4)c4ccccc4n5-c4ccccc4)c4ccc(-c5ccccc5)cc34)cc2)cc1 ; c1ccc(-c2ccc(N(c3ccc4c(c3)c3ccccc3n4-c3ccccc3)c3c4cc(-c5ccccc5)ccc4c(-n4c5ccccc5c5ccccc54)c4cc(-c5ccccc5)ccc34)cc2)cc1]

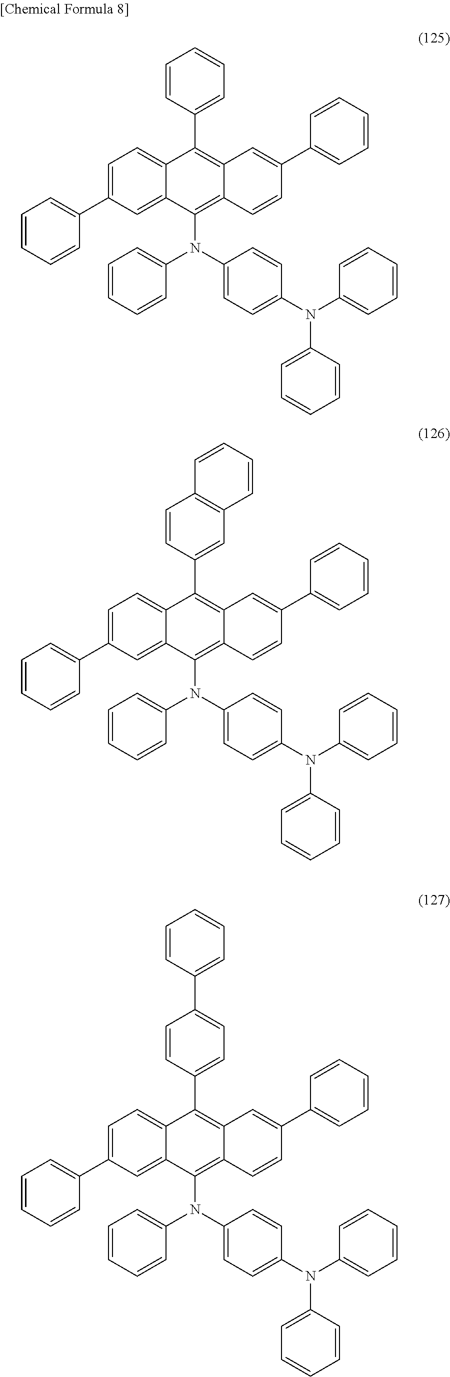 [SMILES: c1ccc(-c2ccc3c(N(c4ccccc4)c4ccc(N(c5ccccc5)c5ccccc5)cc4)c4cc(-c5ccccc5)ccc4c(-c4ccccc4)c3c2)cc1 ; c1ccc(-c2ccc3c(N(c4ccccc4)c4ccc(N(c5ccccc5)c5ccccc5)cc4)c4cc(-c5ccccc5)ccc4c(-c4ccc5ccccc5c4)c3c2)cc1 ; c1ccc(-c2ccc(-c3c4cc(-c5ccccc5)ccc4c(N(c4ccccc4)c4ccc(N(c5ccccc5)c5ccccc5)cc4)c4cc(-c5ccccc5)ccc34)cc2)cc1]

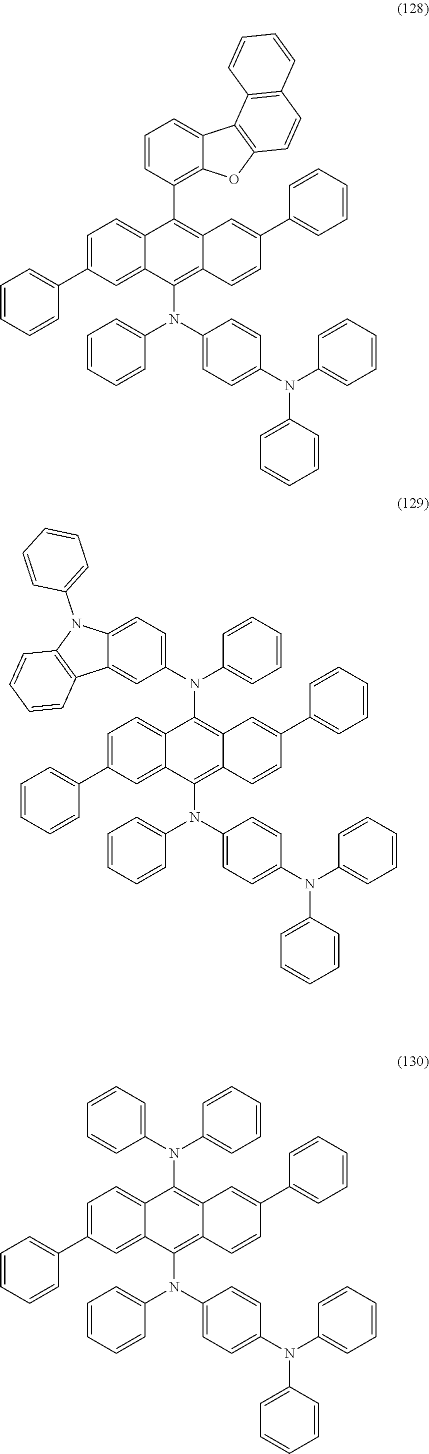 [SMILES: c1ccc(-c2ccc3c(N(c4ccccc4)c4ccc(N(c5ccccc5)c5ccccc5)cc4)c4cc(-c5ccccc5)ccc4c(-c4cccc5c4oc4ccc6ccccc6c45)c3c2)cc1 ; c1ccc(-c2ccc3c(N(c4ccccc4)c4ccc5c(c4)c4ccccc4n5-c4ccccc4)c4cc(-c5ccccc5)ccc4c(N(c4ccccc4)c4ccc(N(c5ccccc5)c5ccccc5)cc4)c3c2)cc1 ; c1ccc(-c2ccc3c(N(c4ccccc4)c4ccc(N(c5ccccc5)c5ccccc5)cc4)c4cc(-c5ccccc5)ccc4c(N(c4ccccc4)c4ccccc4)c3c2)cc1]

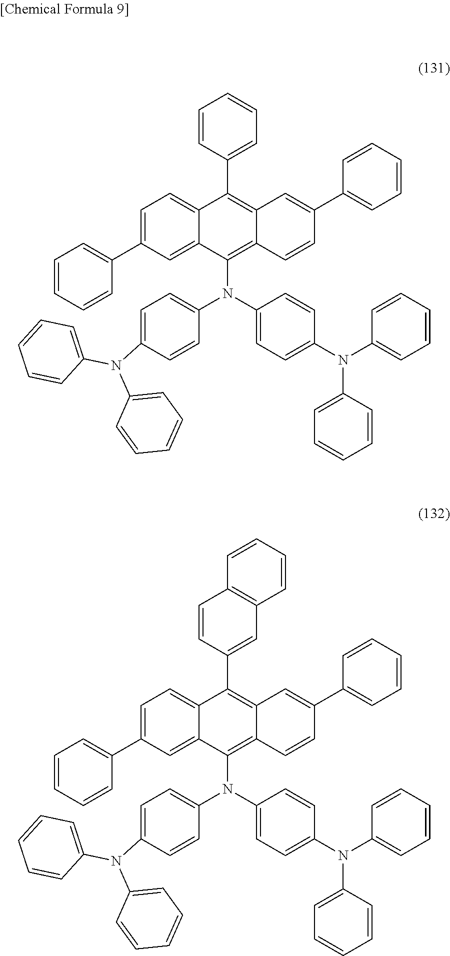 [SMILES: c1ccc(-c2ccc3c(N(c4ccc(N(c5ccccc5)c5ccccc5)cc4)c4ccc(N(c5ccccc5)c5ccccc5)cc4)c4cc(-c5ccccc5)ccc4c(-c4ccccc4)c3c2)cc1 ; c1ccc(-c2ccc3c(N(c4ccc(N(c5ccccc5)c5ccccc5)cc4)c4ccc(N(c5ccccc5)c5ccccc5)cc4)c4cc(-c5ccccc5)ccc4c(-c4ccc5ccccc5c4)c3c2)cc1]

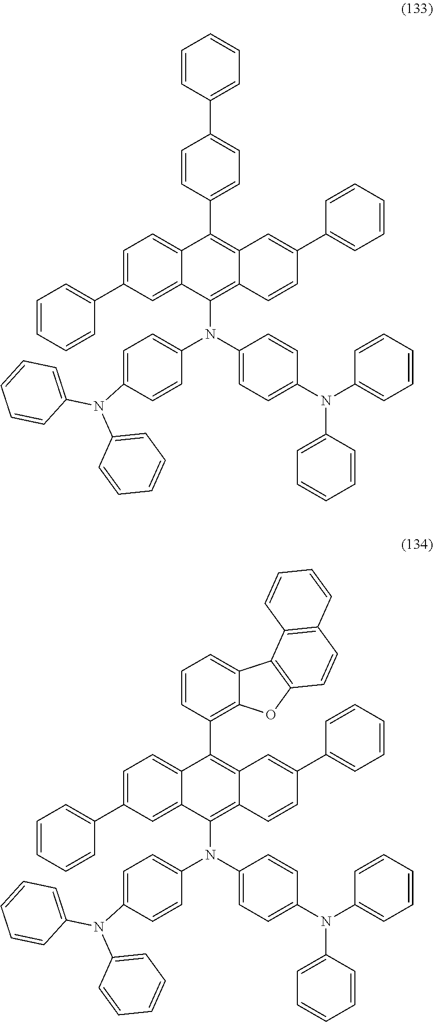 [SMILES: c1ccc(-c2ccc(-c3c4cc(-c5ccccc5)ccc4c(N(c4ccc(N(c5ccccc5)c5ccccc5)cc4)c4ccc(N(c5ccccc5)c5ccccc5)cc4)c4cc(-c5ccccc5)ccc34)cc2)cc1 ; c1ccc(-c2ccc3c(N(c4ccc(N(c5ccccc5)c5ccccc5)cc4)c4ccc(N(c5ccccc5)c5ccccc5)cc4)c4cc(-c5ccccc5)ccc4c(-c4cccc5c4oc4ccc6ccccc6c45)c3c2)cc1]

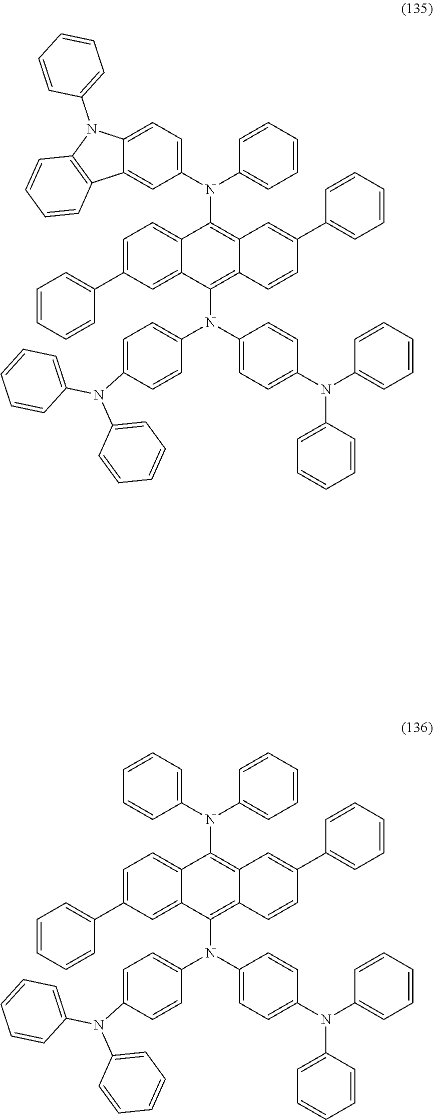 [SMILES: c1ccc(-c2ccc3c(N(c4ccccc4)c4ccc5c(c4)c4ccccc4n5-c4ccccc4)c4cc(-c5ccccc5)ccc4c(N(c4ccc(N(c5ccccc5)c5ccccc5)cc4)c4ccc(N(c5ccccc5)c5ccccc5)cc4)c3c2)cc1 ; c1ccc(-c2ccc3c(N(c4ccc(N(c5ccccc5)c5ccccc5)cc4)c4ccc(N(c5ccccc5)c5ccccc5)cc4)c4cc(-c5ccccc5)ccc4c(N(c4ccccc4)c4ccccc4)c3c2)cc1]

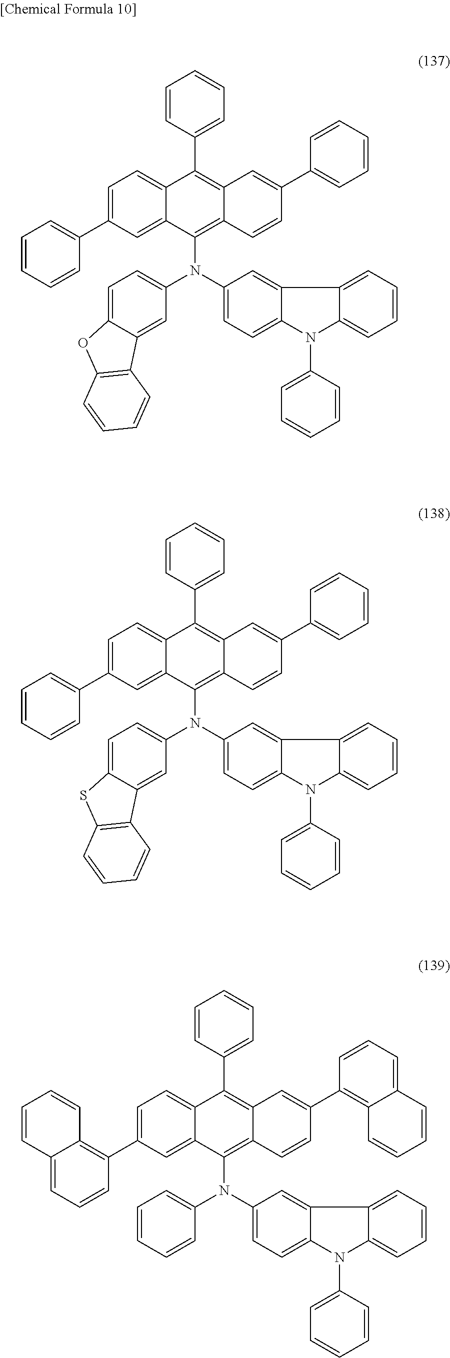 [SMILES: c1ccc(-c2ccc3c(N(c4ccc5oc6ccccc6c5c4)c4ccc5c(c4)c4ccccc4n5-c4ccccc4)c4cc(-c5ccccc5)ccc4c(-c4ccccc4)c3c2)cc1 ; c1ccc(-c2ccc3c(N(c4ccc5sc6ccccc6c5c4)c4ccc5c(c4)c4ccccc4n5-c4ccccc4)c4cc(-c5ccccc5)ccc4c(-c4ccccc4)c3c2)cc1 ; c1ccc(-c2c3cc(-c4cccc5ccccc45)ccc3c(N(c3ccccc3)c3ccc4c(c3)c3ccccc3n4-c3ccccc3)c3cc(-c4cccc5ccccc45)ccc23)cc1]

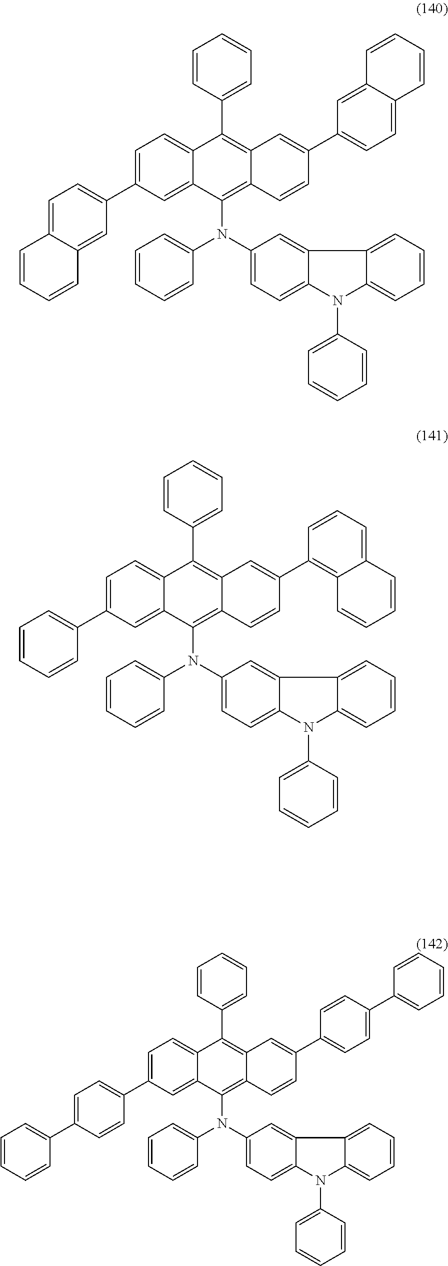 [SMILES: c1ccc(-c2c3cc(-c4ccc5ccccc5c4)ccc3c(N(c3ccccc3)c3ccc4c(c3)c3ccccc3n4-c3ccccc3)c3cc(-c4ccc5ccccc5c4)ccc23)cc1 ; c1ccc(-c2ccc3c(-c4ccccc4)c4cc(-c5cccc6ccccc56)ccc4c(N(c4ccccc4)c4ccc5c(c4)c4ccccc4n5-c4ccccc4)c3c2)cc1 ; c1ccc(-c2ccc(-c3ccc4c(N(c5ccccc5)c5ccc6c(c5)c5ccccc5n6-c5ccccc5)c5cc(-c6ccc(-c7ccccc7)cc6)ccc5c(-c5ccccc5)c4c3)cc2)cc1]

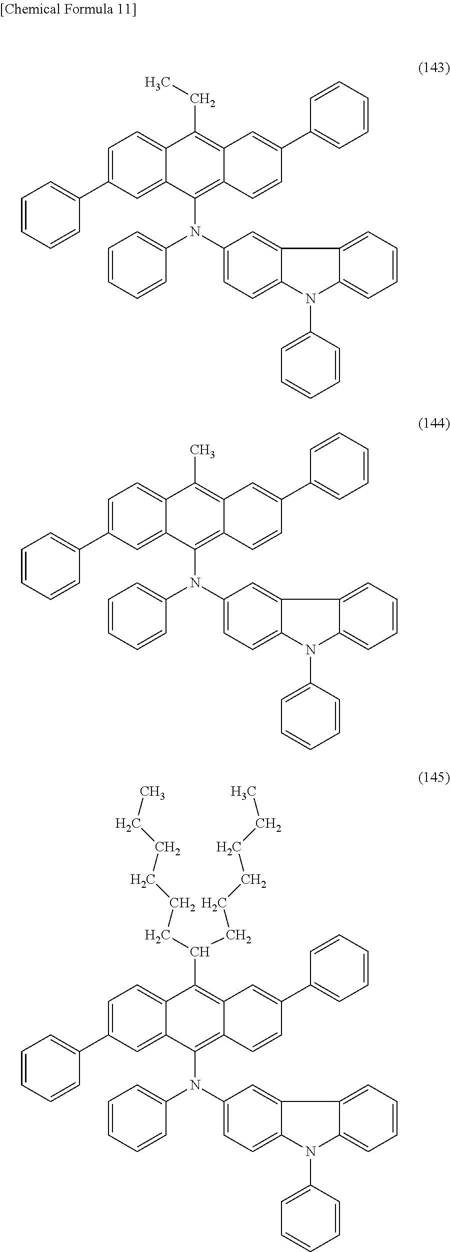

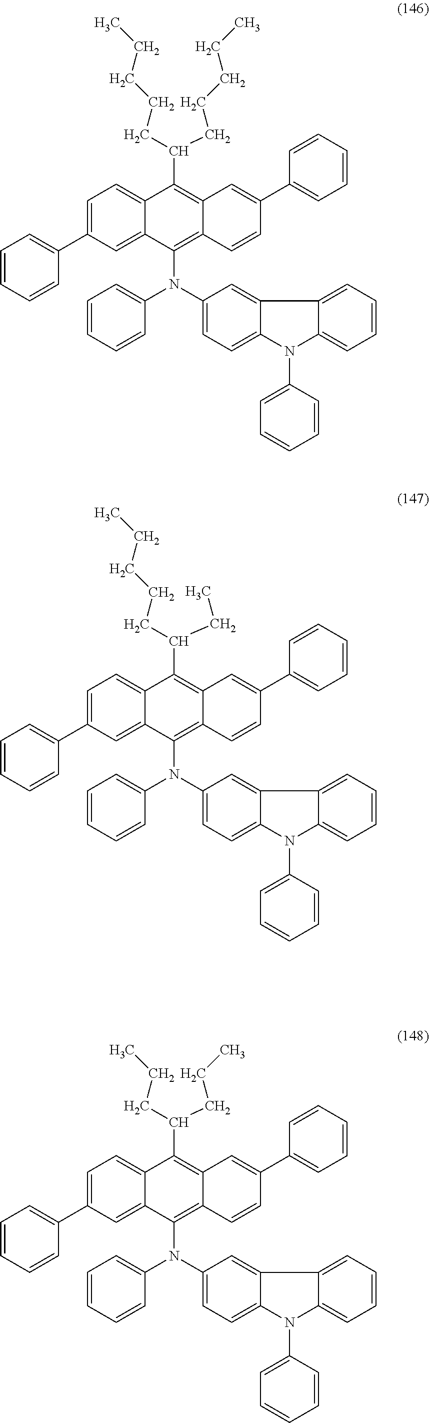 [SMILES: CCCCCC(CCCCC)c1c2cc(-c3ccccc3)ccc2c(N(c2ccccc2)c2ccc3c(c2)c2ccccc2n3-c2ccccc2)c2cc(-c3ccccc3)ccc12 ; CCCCCC(CC)c1c2cc(-c3ccccc3)ccc2c(N(c2ccccc2)c2ccc3c(c2)c2ccccc2n3-c2ccccc2)c2cc(-c3ccccc3)ccc12 ; CCCC(CCC)c1c2cc(-c3ccccc3)ccc2c(N(c2ccccc2)c2ccc3c(c2)c2ccccc2n3-c2ccccc2)c2cc(-c3ccccc3)ccc12]

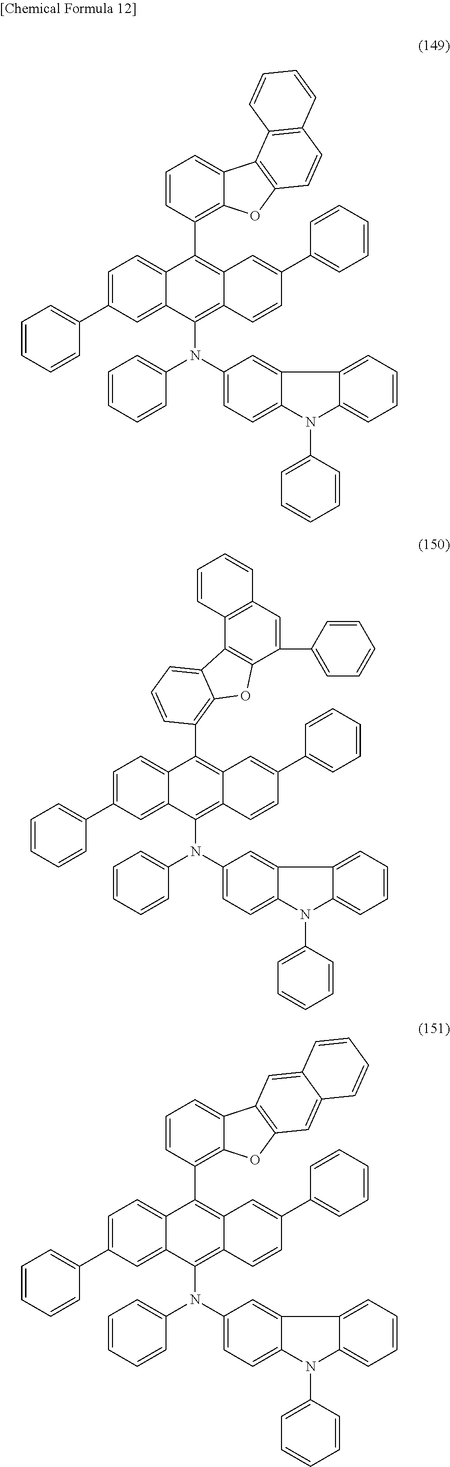

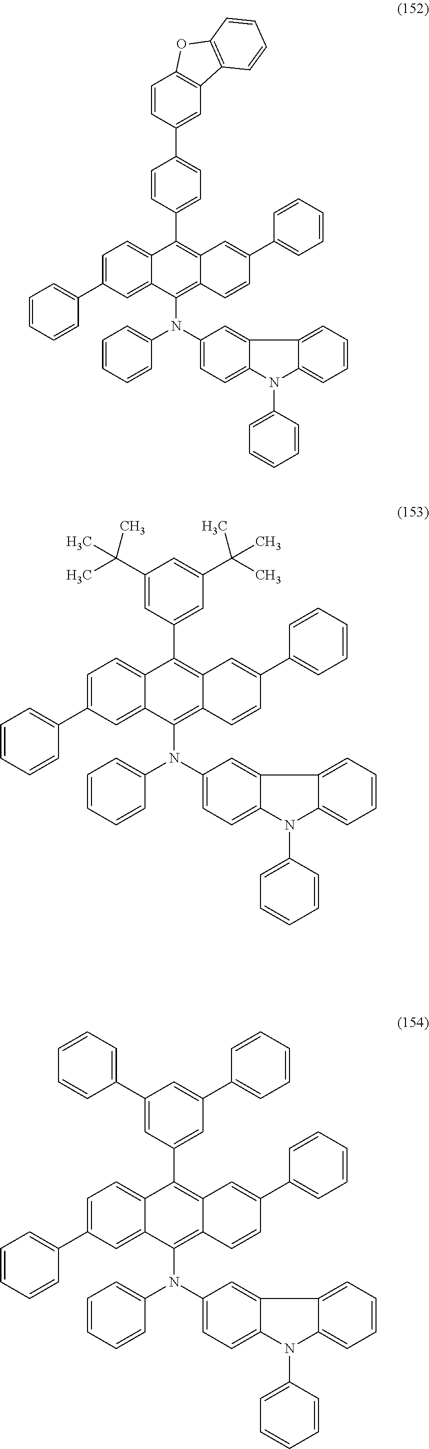 [SMILES: c1ccc(-c2ccc3c(N(c4ccccc4)c4ccc5c(c4)c4ccccc4n5-c4ccccc4)c4cc(-c5ccccc5)ccc4c(-c4ccc(-c5ccc6oc7ccccc7c6c5)cc4)c3c2)cc1 ; CC(C)(C)c1cc(-c2c3cc(-c4ccccc4)ccc3c(N(c3ccccc3)c3ccc4c(c3)c3ccccc3n4-c3ccccc3)c3cc(-c4ccccc4)ccc23)cc(C(C)(C)C)c1 ; c1ccc(-c2cc(-c3ccccc3)cc(-c3c4cc(-c5ccccc5)ccc4c(N(c4ccccc4)c4ccc5c(c4)c4ccccc4n5-c4ccccc4)c4cc(-c5ccccc5)ccc34)c2)cc1]

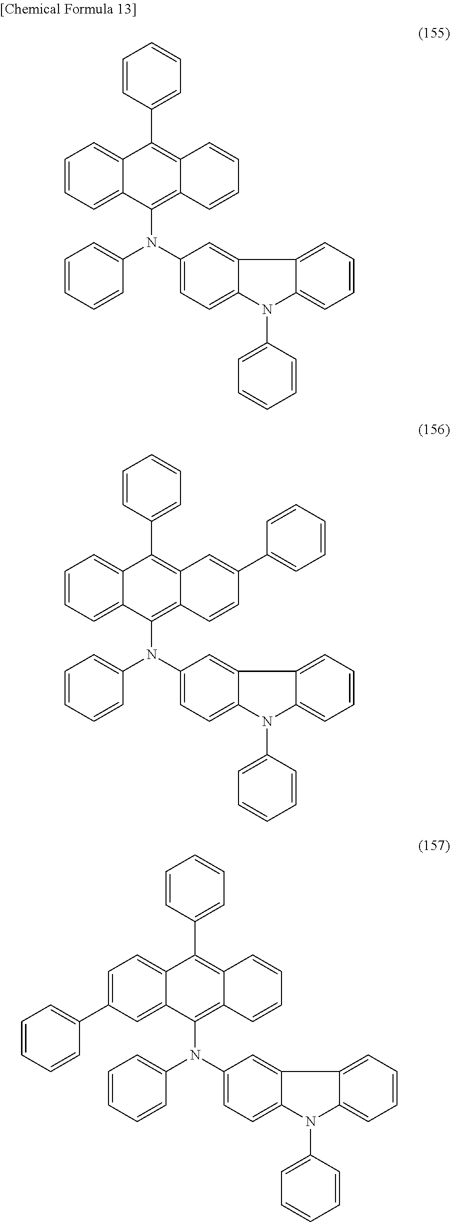 [SMILES: c1ccc(-c2c3ccccc3c(N(c3ccccc3)c3ccc4c(c3)c3ccccc3n4-c3ccccc3)c3ccccc23)cc1 ; c1ccc(-c2ccc3c(N(c4ccccc4)c4ccc5c(c4)c4ccccc4n5-c4ccccc4)c4ccccc4c(-c4ccccc4)c3c2)cc1 ; c1ccc(-c2ccc3c(-c4ccccc4)c4ccccc4c(N(c4ccccc4)c4ccc5c(c4)c4ccccc4n5-c4ccccc4)c3c2)cc1]

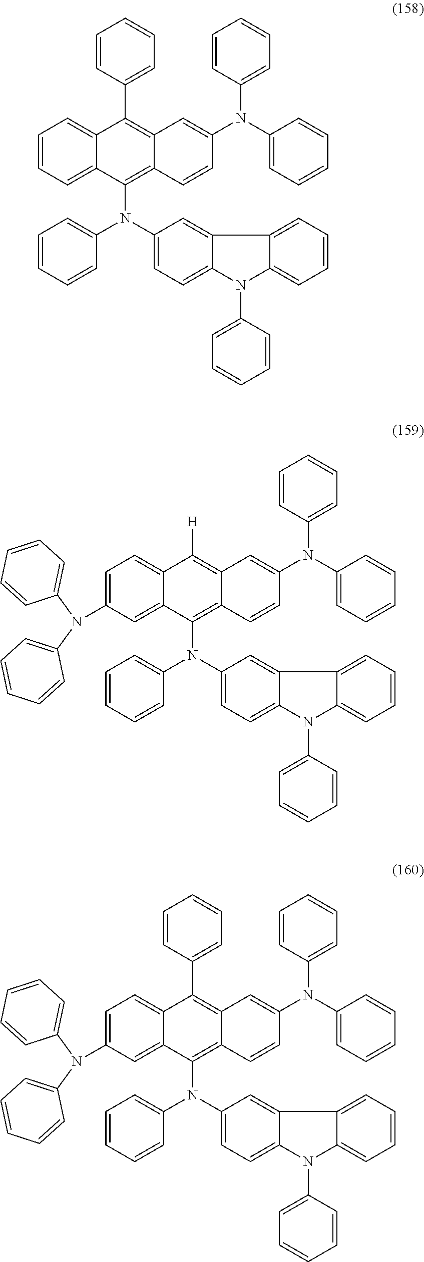 [SMILES: c1ccc(-c2c3ccccc3c(N(c3ccccc3)c3ccc4c(c3)c3ccccc3n4-c3ccccc3)c3ccc(N(c4ccccc4)c4ccccc4)cc23)cc1 ; c1ccc(N(c2ccccc2)c2ccc3c(N(c4ccccc4)c4ccc5c(c4)c4ccccc4n5-c4ccccc4)c4cc(N(c5ccccc5)c5ccccc5)ccc4cc3c2)cc1 ; c1ccc(-c2c3cc(N(c4ccccc4)c4ccccc4)ccc3c(N(c3ccccc3)c3ccc4c(c3)c3ccccc3n4-c3ccccc3)c3cc(N(c4ccccc4)c4ccccc4)ccc23)cc1]

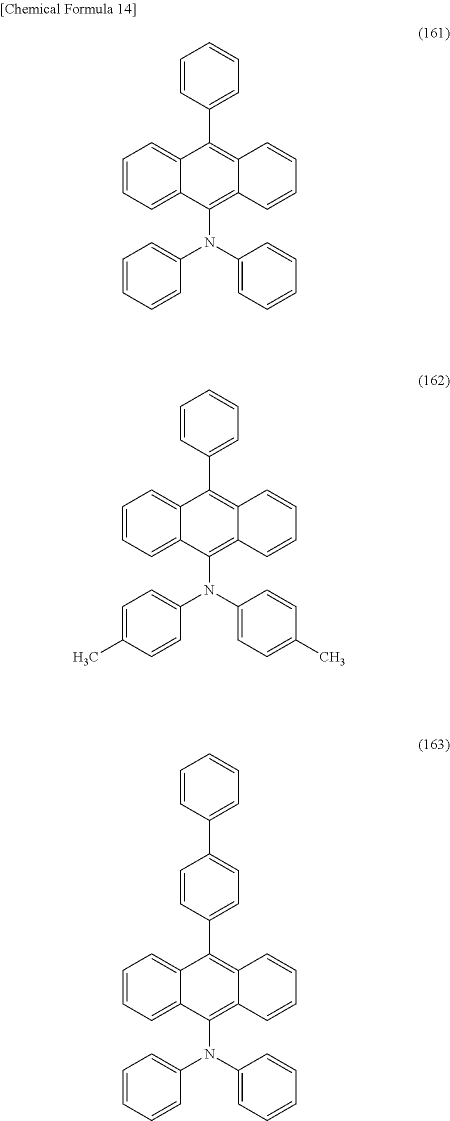

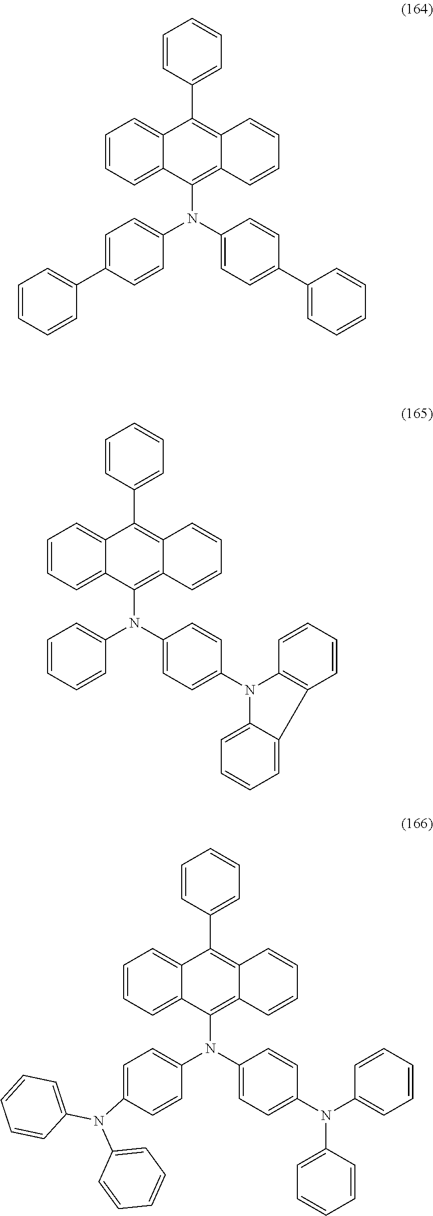 [SMILES: c1ccc(-c2ccc(N(c3ccc(-c4ccccc4)cc3)c3c4ccccc4c(-c4ccccc4)c4ccccc34)cc2)cc1 ; c1ccc(-c2c3ccccc3c(N(c3ccccc3)c3ccc(-n4c5ccccc5c5ccccc54)cc3)c3ccccc23)cc1 ; c1ccc(-c2c3ccccc3c(N(c3ccc(N(c4ccccc4)c4ccccc4)cc3)c3ccc(N(c4ccccc4)c4ccccc4)cc3)c3ccccc23)cc1]

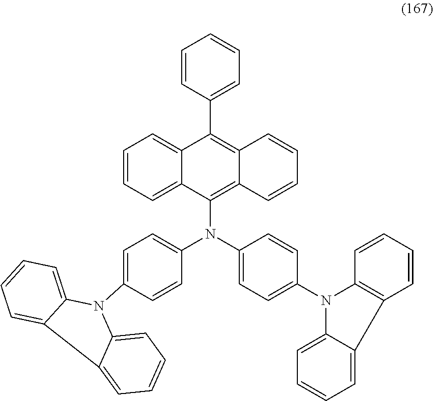 [SMILES: c1ccc(-c2c3ccccc3c(N(c3ccc(-n4c5ccccc5c5ccccc54)cc3)c3ccc(-n4c5ccccc5c5ccccc54)cc3)c3ccccc23)cc1]

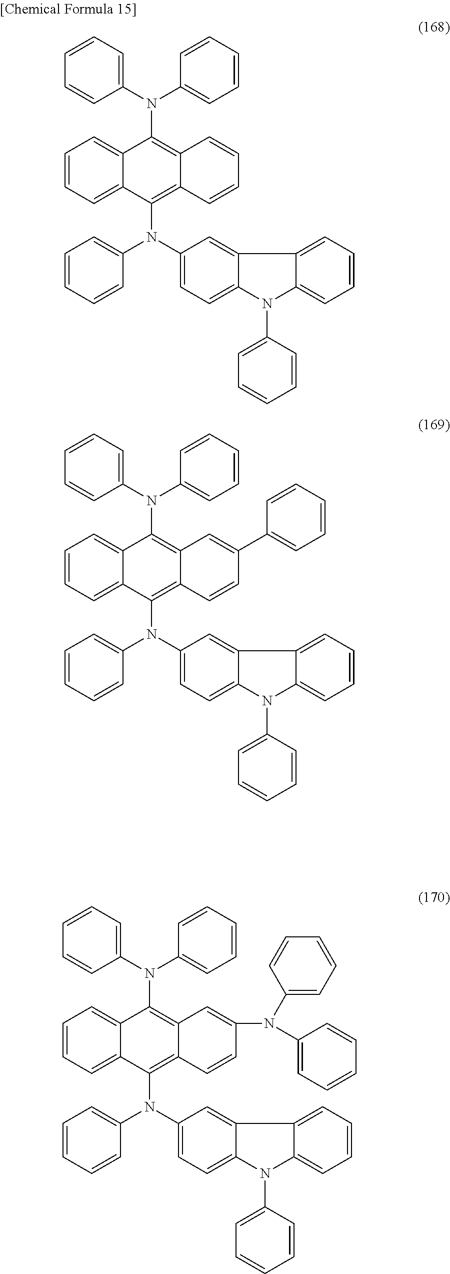 [SMILES: c1ccc(N(c2ccccc2)c2c3ccccc3c(N(c3ccccc3)c3ccc4c(c3)c3ccccc3n4-c3ccccc3)c3ccccc23)cc1 ; c1ccc(-c2ccc3c(N(c4ccccc4)c4ccc5c(c4)c4ccccc4n5-c4ccccc4)c4ccccc4c(N(c4ccccc4)c4ccccc4)c3c2)cc1 ; c1ccc(N(c2ccccc2)c2ccc3c(N(c4ccccc4)c4ccc5c(c4)c4ccccc4n5-c4ccccc4)c4ccccc4c(N(c4ccccc4)c4ccccc4)c3c2)cc1]

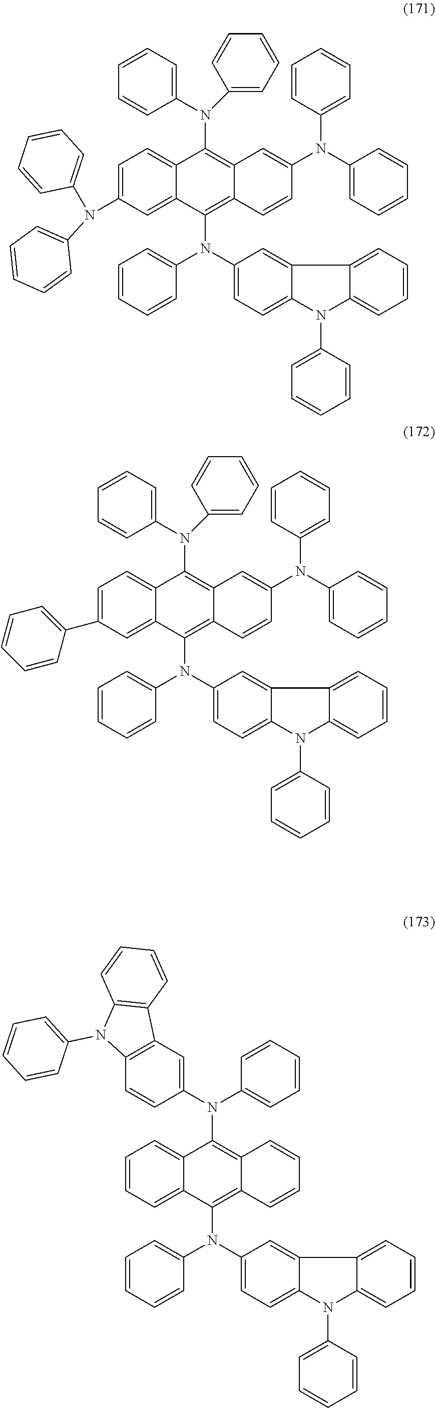 [SMILES: c1ccc(N(c2ccccc2)c2ccc3c(N(c4ccccc4)c4ccc5c(c4)c4ccccc4n5-c4ccccc4)c4cc(N(c5ccccc5)c5ccccc5)ccc4c(N(c4ccccc4)c4ccccc4)c3c2)cc1 ; c1ccc(-c2ccc3c(N(c4ccccc4)c4ccccc4)c4cc(N(c5ccccc5)c5ccccc5)ccc4c(N(c4ccccc4)c4ccc5c(c4)c4ccccc4n5-c4ccccc4)c3c2)cc1 ; c1ccc(N(c2ccc3c(c2)c2ccccc2n3-c2ccccc2)c2c3ccccc3c(N(c3ccccc3)c3ccc4c(c3)c3ccccc3n4-c3ccccc3)c3ccccc23)cc1]

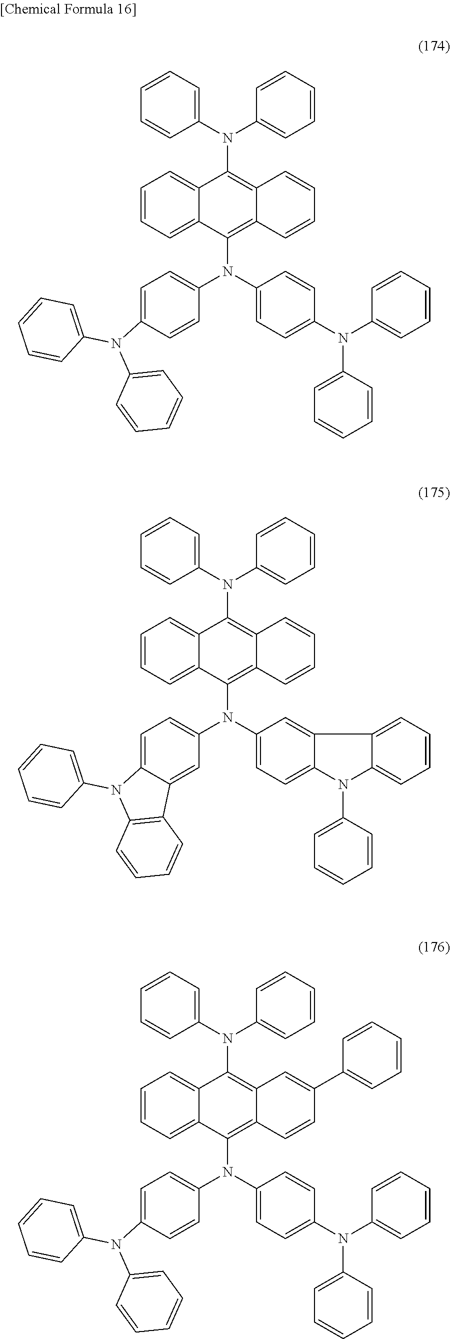 [SMILES: c1ccc(N(c2ccccc2)c2ccc(N(c3ccc(N(c4ccccc4)c4ccccc4)cc3)c3c4ccccc4c(N(c4ccccc4)c4ccccc4)c4ccccc34)cc2)cc1 ; c1ccc(N(c2ccccc2)c2c3ccccc3c(N(c3ccc4c(c3)c3ccccc3n4-c3ccccc3)c3ccc4c(c3)c3ccccc3n4-c3ccccc3)c3ccccc23)cc1 ; c1ccc(-c2ccc3c(N(c4ccc(N(c5ccccc5)c5ccccc5)cc4)c4ccc(N(c5ccccc5)c5ccccc5)cc4)c4ccccc4c(N(c4ccccc4)c4ccccc4)c3c2)cc1]

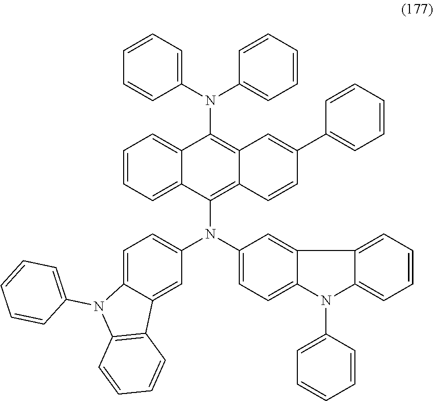 [SMILES: c1ccc(-c2ccc3c(N(c4ccc5c(c4)c4ccccc4n5-c4ccccc4)c4ccc5c(c4)c4ccccc4n5-c4ccccc4)c4ccccc4c(N(c4ccccc4)c4ccccc4)c3c2)cc1]

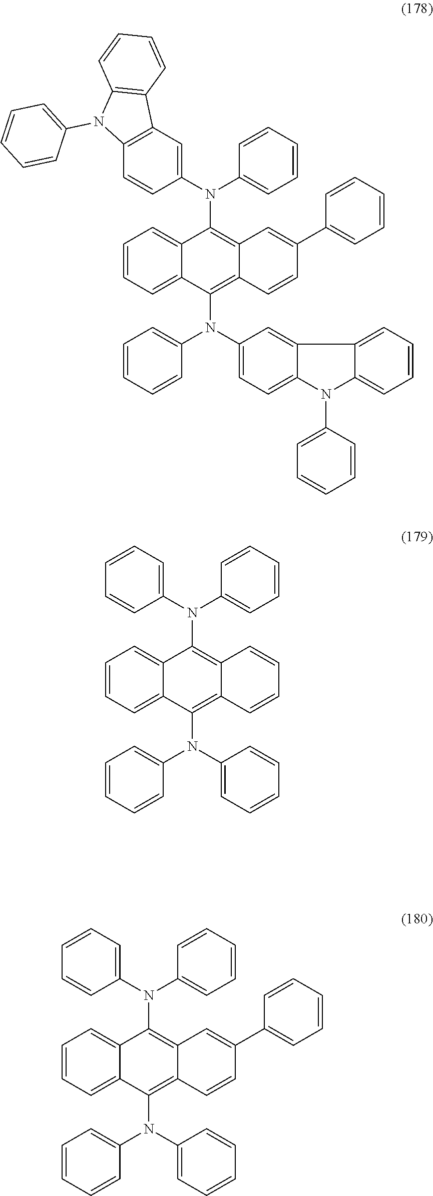 [SMILES: c1ccc(-c2ccc3c(N(c4ccccc4)c4ccc5c(c4)c4ccccc4n5-c4ccccc4)c4ccccc4c(N(c4ccccc4)c4ccc5c(c4)c4ccccc4n5-c4ccccc4)c3c2)cc1 ; c1ccc(N(c2ccccc2)c2c3ccccc3c(N(c3ccccc3)c3ccccc3)c3ccccc23)cc1 ; c1ccc(-c2ccc3c(N(c4ccccc4)c4ccccc4)c4ccccc4c(N(c4ccccc4)c4ccccc4)c3c2)cc1]

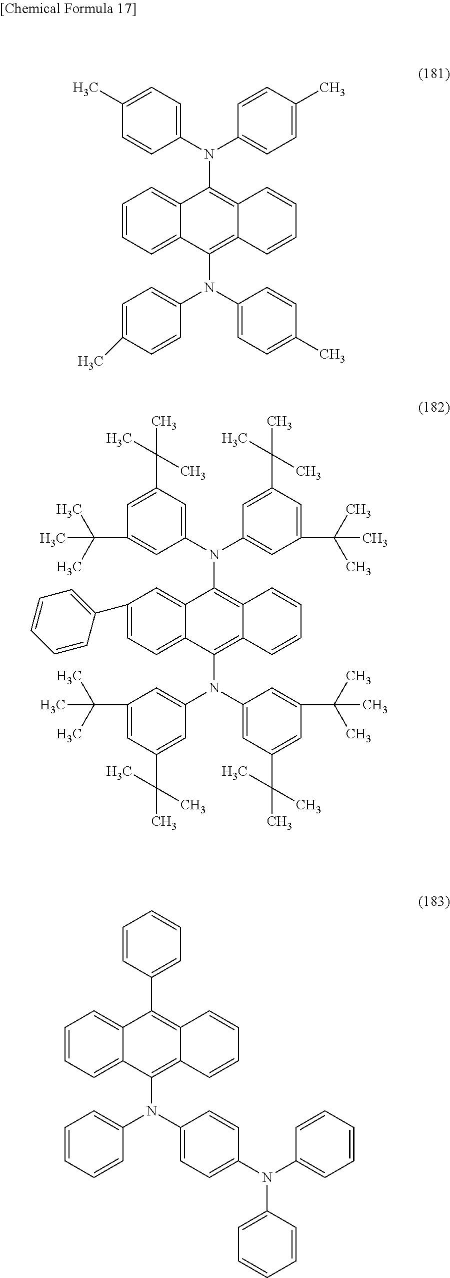

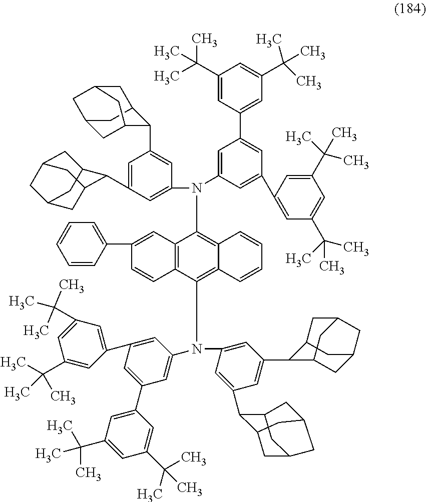 [SMILES: CC(C)(C)c1cc(-c2cc(-c3cc(C(C)(C)C)cc(C(C)(C)C)c3)cc(N(c3cc(C4C5CC6CC(C5)CC4C6)cc(C4C5CC6CC(C5)CC4C6)c3)c3c4ccccc4c(N(c4cc(-c5cc(C(C)(C)C)cc(C(C)(C)C)c5)cc(-c5cc(C(C)(C)C)cc(C(C)(C)C)c5)c4)c4cc(C5C6CC7CC(C6)CC5C7)cc(C5C6CC7CC(C6)CC5C7)c4)c4cc(-c5ccccc5)ccc34)c2)cc(C(C)(C)C)c1]

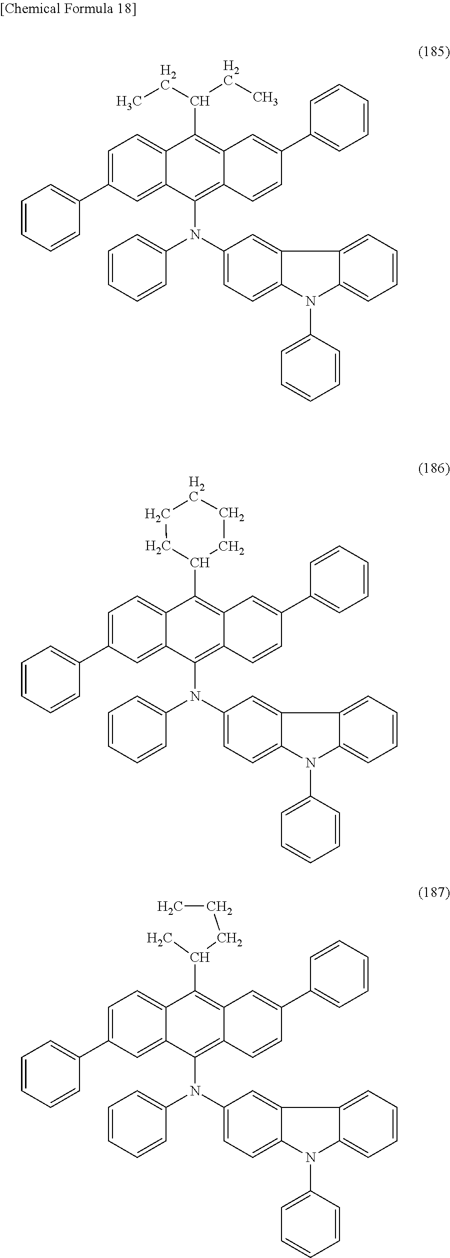 [SMILES: CCC(CC)c1c2cc(-c3ccccc3)ccc2c(N(c2ccccc2)c2ccc3c(c2)c2ccccc2n3-c2ccccc2)c2cc(-c3ccccc3)ccc12 ; c1ccc(-c2ccc3c(N(c4ccccc4)c4ccc5c(c4)c4ccccc4n5-c4ccccc4)c4cc(-c5ccccc5)ccc4c(C4CCCCC4)c3c2)cc1 ; CCCC(C)c1c2cc(-c3ccccc3)ccc2c(N(c2ccccc2)c2ccc3c(c2)c2ccccc2n3-c2ccccc2)c2cc(-c3ccccc3)ccc12]

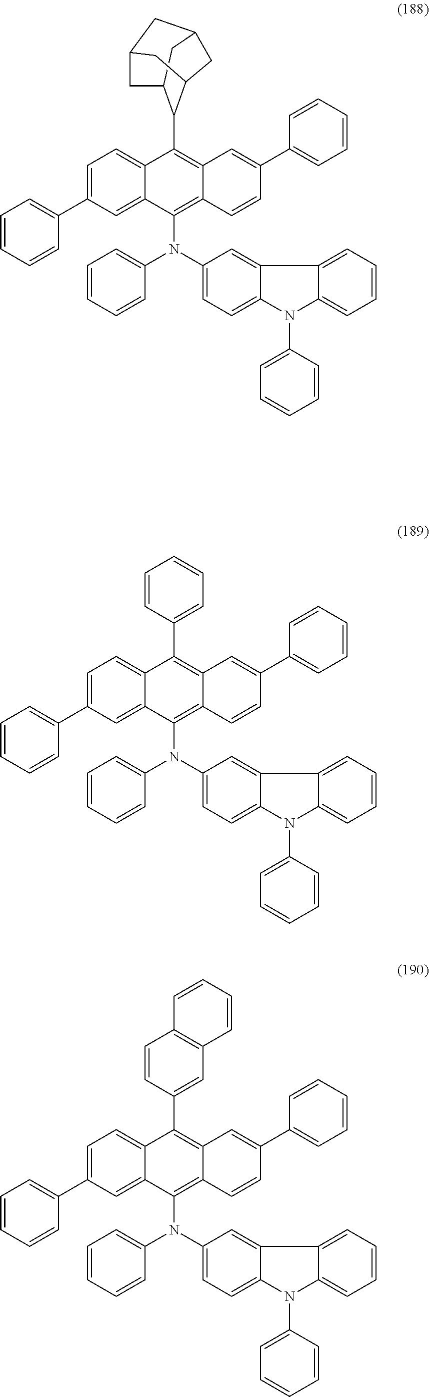 [SMILES: c1ccc(-c2ccc3c(N(c4ccccc4)c4ccc5c(c4)c4ccccc4n5-c4ccccc4)c4cc(-c5ccccc5)ccc4c(C4C5CC6CC(C5)CC4C6)c3c2)cc1 ; c1ccc(-c2ccc3c(N(c4ccccc4)c4ccc5c(c4)c4ccccc4n5-c4ccccc4)c4cc(-c5ccccc5)ccc4c(-c4ccccc4)c3c2)cc1 ; c1ccc(-c2ccc3c(N(c4ccccc4)c4ccc5c(c4)c4ccccc4n5-c4ccccc4)c4cc(-c5ccccc5)ccc4c(-c4ccc5ccccc5c4)c3c2)cc1]

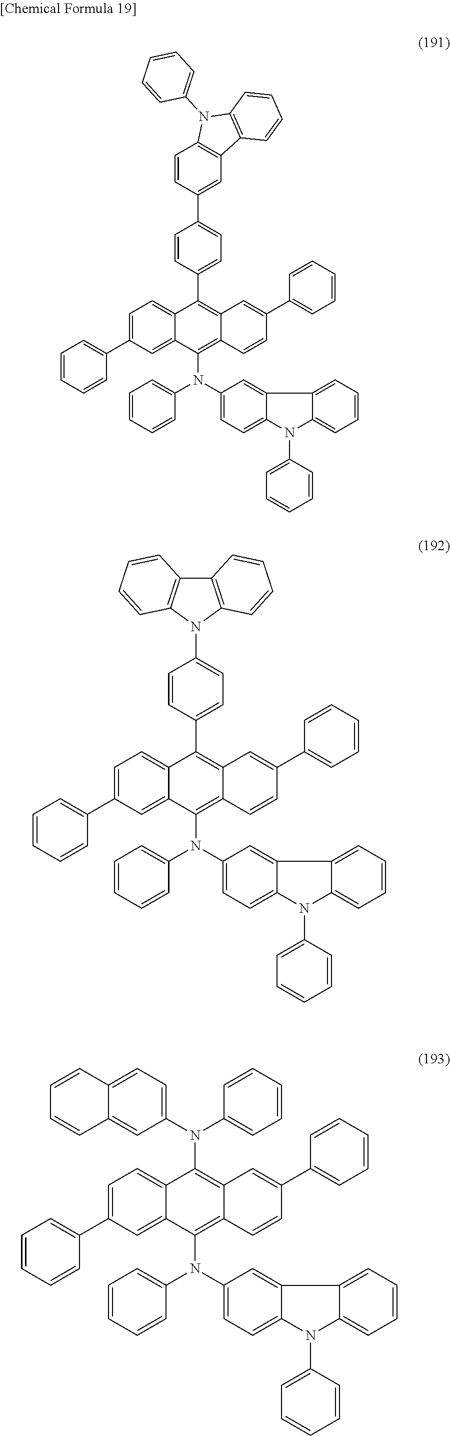 [SMILES: c1ccc(-c2ccc3c(N(c4ccccc4)c4ccc5c(c4)c4ccccc4n5-c4ccccc4)c4cc(-c5ccccc5)ccc4c(-c4ccc(-c5ccc6c(c5)c5ccccc5n6-c5ccccc5)cc4)c3c2)cc1 ; c1ccc(-c2ccc3c(N(c4ccccc4)c4ccc5c(c4)c4ccccc4n5-c4ccccc4)c4cc(-c5ccccc5)ccc4c(-c4ccc(-n5c6ccccc6c6ccccc65)cc4)c3c2)cc1 ; c1ccc(-c2ccc3c(N(c4ccccc4)c4ccc5c(c4)c4ccccc4n5-c4ccccc4)c4cc(-c5ccccc5)ccc4c(N(c4ccccc4)c4ccc5ccccc5c4)c3c2)cc1]

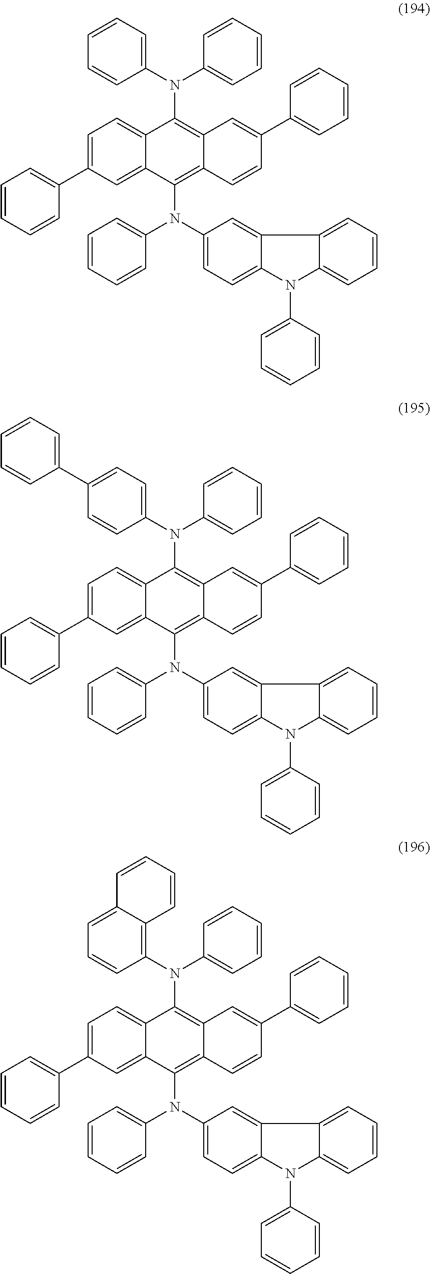 [SMILES: c1ccc(-c2ccc3c(N(c4ccccc4)c4ccc5c(c4)c4ccccc4n5-c4ccccc4)c4cc(-c5ccccc5)ccc4c(N(c4ccccc4)c4ccccc4)c3c2)cc1 ; c1ccc(-c2ccc(N(c3ccccc3)c3c4ccc(-c5ccccc5)cc4c(N(c4ccccc4)c4ccc5c(c4)c4ccccc4n5-c4ccccc4)c4ccc(-c5ccccc5)cc34)cc2)cc1 ; c1ccc(-c2ccc3c(N(c4ccccc4)c4cccc5ccccc45)c4cc(-c5ccccc5)ccc4c(N(c4ccccc4)c4ccc5c(c4)c4ccccc4n5-c4ccccc4)c3c2)cc1]

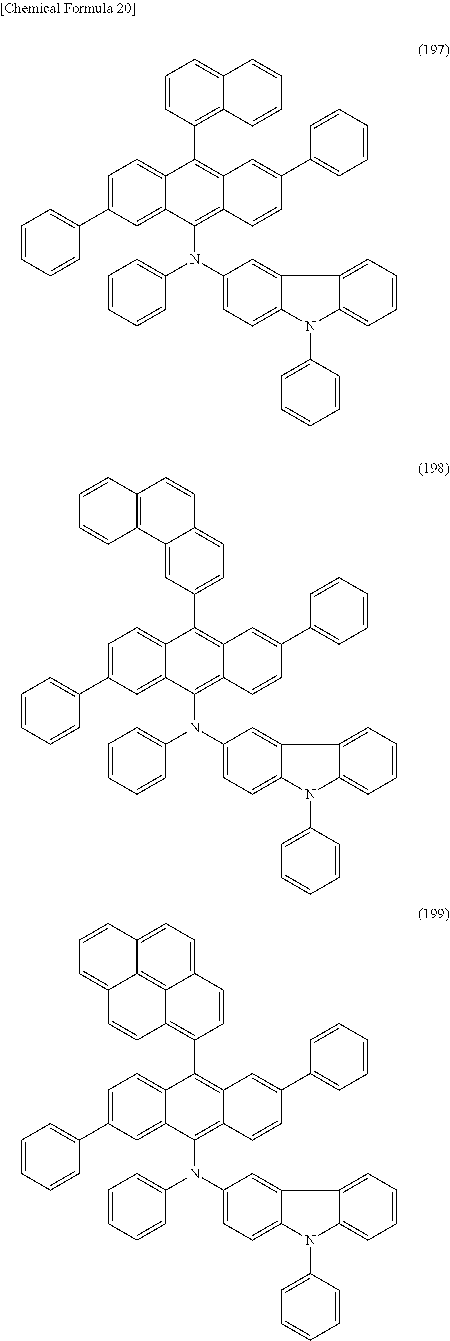 [SMILES: c1ccc(-c2ccc3c(N(c4ccccc4)c4ccc5c(c4)c4ccccc4n5-c4ccccc4)c4cc(-c5ccccc5)ccc4c(-c4cccc5ccccc45)c3c2)cc1 ; c1ccc(-c2ccc3c(N(c4ccccc4)c4ccc5c(c4)c4ccccc4n5-c4ccccc4)c4cc(-c5ccccc5)ccc4c(-c4ccc5ccc6ccccc6c5c4)c3c2)cc1 ; c1ccc(-c2ccc3c(N(c4ccccc4)c4ccc5c(c4)c4ccccc4n5-c4ccccc4)c4cc(-c5ccccc5)ccc4c(-c4ccc5ccc6cccc7ccc4c5c67)c3c2)cc1]

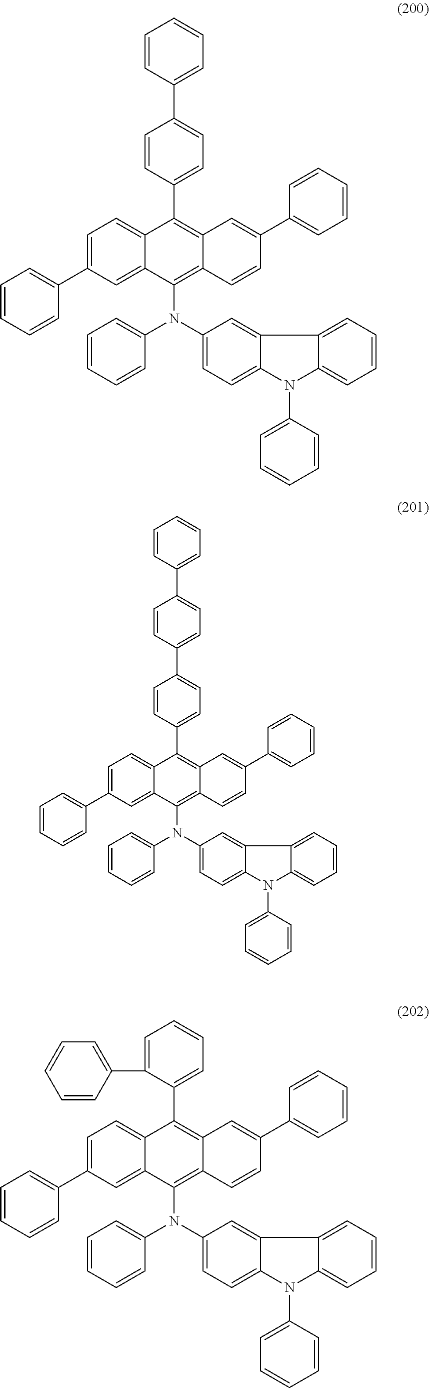 [SMILES: c1ccc(-c2ccc(-c3c4cc(-c5ccccc5)ccc4c(N(c4ccccc4)c4ccc5c(c4)c4ccccc4n5-c4ccccc4)c4cc(-c5ccccc5)ccc34)cc2)cc1 ; c1ccc(-c2ccc(-c3ccc(-c4c5cc(-c6ccccc6)ccc5c(N(c5ccccc5)c5ccc6c(c5)c5ccccc5n6-c5ccccc5)c5cc(-c6ccccc6)ccc45)cc3)cc2)cc1 ; c1ccc(-c2ccc3c(N(c4ccccc4)c4ccc5c(c4)c4ccccc4n5-c4ccccc4)c4cc(-c5ccccc5)ccc4c(-c4ccccc4-c4ccccc4)c3c2)cc1]

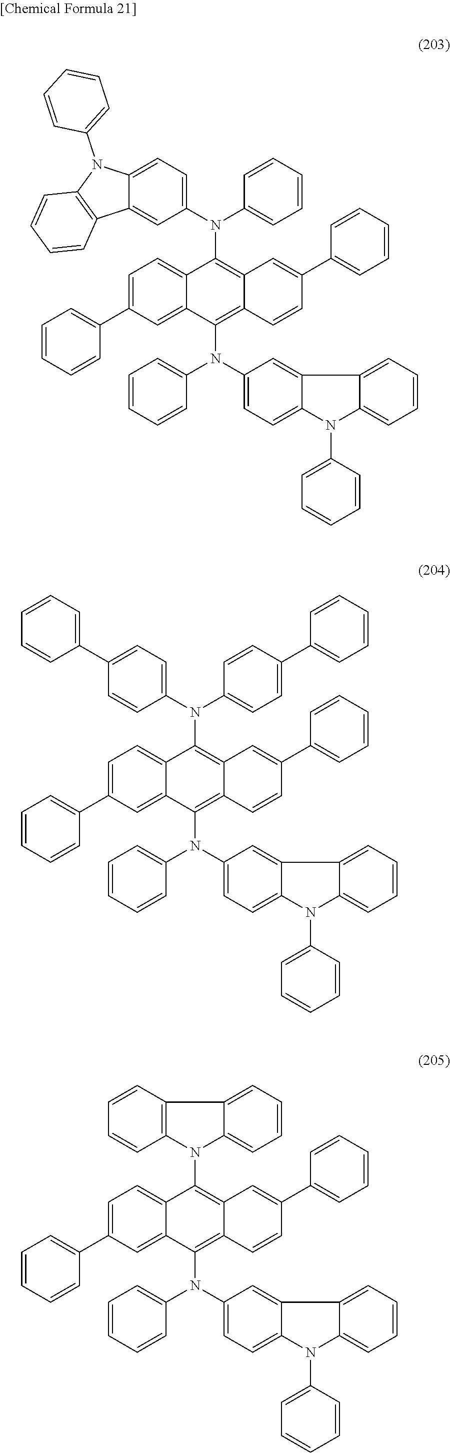 [SMILES: c1ccc(-c2ccc3c(N(c4ccccc4)c4ccc5c(c4)c4ccccc4n5-c4ccccc4)c4cc(-c5ccccc5)ccc4c(N(c4ccccc4)c4ccc5c(c4)c4ccccc4n5-c4ccccc4)c3c2)cc1 ; c1ccc(-c2ccc(N(c3ccc(-c4ccccc4)cc3)c3c4ccc(-c5ccccc5)cc4c(N(c4ccccc4)c4ccc5c(c4)c4ccccc4n5-c4ccccc4)c4ccc(-c5ccccc5)cc34)cc2)cc1 ; c1ccc(-c2ccc3c(-n4c5ccccc5c5ccccc54)c4cc(-c5ccccc5)ccc4c(N(c4ccccc4)c4ccc5c(c4)c4ccccc4n5-c4ccccc4)c3c2)cc1]

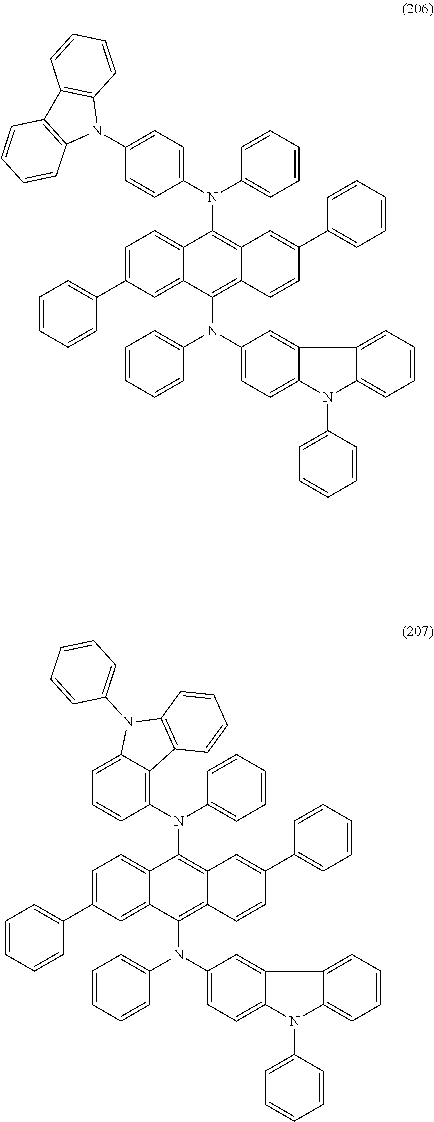 [SMILES: c1ccc(-c2ccc3c(N(c4ccccc4)c4ccc5c(c4)c4ccccc4n5-c4ccccc4)c4cc(-c5ccccc5)ccc4c(N(c4ccccc4)c4ccc(-n5c6ccccc6c6ccccc65)cc4)c3c2)cc1 ; c1ccc(-c2ccc3c(N(c4ccccc4)c4cccc5c4c4ccccc4n5-c4ccccc4)c4cc(-c5ccccc5)ccc4c(N(c4ccccc4)c4ccc5c(c4)c4ccccc4n5-c4ccccc4)c3c2)cc1]

This embodiment can be combined with any of the other embodiments and examples as appropriate.

### Embodiment 2

In this embodiment, a structure of the photoelectric conversion device 550S of one embodiment of the present invention will be described with reference to FIG. 1A and FIG. 1B.

FIG. 1A is a cross-sectional view illustrating a structure of the photoelectric conversion device of one embodiment of the present invention, and FIG. 1B is a cross-sectional view illustrating a structure of the photoelectric conversion device of one embodiment of the present invention which is different from the structure in FIG. 1A.

### <Structure Example of Photoelectric Conversion Device 550S>

The photoelectric conversion device 550S described in this embodiment includes the electrode 551S, the electrode 552S, and an unit 103S. The electrode 552S overlaps with the electrode 551S, and the unit 103S is held between the electrode 552S and the electrode 551S.

### <Structure Example of Unit 103S>

The unit 103S absorbs light hv, supplies electrons to one electrode, and supplies holes to the other electrode. For example, the unit 103S supplies holes to the electrode 551S and supplies electrons to the electrode 552S.

The unit 103S has a single-layer structure or a stacked-layer structure. For example, the unit 103S includes the layer 114S, the layer 112, and the layer 113 (see FIG. 1A). The layer 114S is held between the layer 112 and the layer 113, the layer 112 is held between the electrode 551S and the layer 114S, and the layer 113 is held between the electrode 552S and the layer 114S.

For example, a layer selected from functional layers such as a photoelectric conversion layer, a hole-transport layer, an electron-transport layer, and a carrier-blocking layer can be used in the unit 103S.

### <<Structure Example 1 of Layer 114S>>

A layer 114S(i,j) can be referred to as a photoelectric conversion layer. The layer 114S(i,j) absorbs the light hv, supplies electrons to a layer in contact with one surface of the layer 114S(i,j), and supplies holes to a layer in contact with the other surface of the layer 114S(i,j). For example, the layer 114S(i,j) supplies holes to the layer 112 and supplies electrons to the layer 113. For example, a material usable for an organic solar cell can be used for the layer 114S(i,j). Specifically, an electron-accepting material and an electron-donating material can be used for the layer 114S(i,j).

### [Example of Electron-Accepting Material]

As the electron-accepting material, a fullerene derivative or a non-fullerene electron acceptor can be used, for example.

As the electron-accepting material, a C₆₀ fullerene, a C₇₀ fullerene, [6,6]-phenyl-C₇₁-butyric acid methyl ester (abbreviation: PC71BM), [6,6]-phenyl-C₆₁-butyric acid methyl ester (abbreviation: PC61BM), 1′,1″,4′,4″-tetrohydro-di[1,4]methanonaphthaleno[1,2:2′,3′,56,60:2″,3″ ][5,6]fullerene-C₆₀ (abbreviation: ICBA), or the like can be used, for example.

As the non-fullerene electron acceptor, a perylene derivative, a compound having a dicyanomethyleneindanone group, or the like can be used. For example, N,N′-dimethyl-3,4,9,10-perylenedicarboximide (abbreviation: Me-PTCDI) can be used.

### [Example 1 of Electron-Donating Material]

As the electron-donating material, the photoelectric conversion device material of one embodiment of the present invention described in Embodiment 1 can be used, for example.

As the electron-donating material, N-phenyl-N-[4-(diphenylamino)-phenyl]-10-phenyl-9-anthracenamine (abbreviation: DPAPhA), 9,10-bis[N,N-di-(p-tolyl)-amino]anthracene (abbreviation: TTPA), N,N′-(2-phenylanthracene-9,10-diyl)-N,N,N′,N′-tetrakis(3,5-di-tert-butylphenyl)diamine (abbreviation: 2Ph-mmtBuDPhA2Anth), or N,N′-bis[3,5-bis(2-adamantyl)phenyl]-N,N′-bis[3,5-bis(3,5-di-tert-butylphenyl)phenyl]-2-phenylanthracene-9,10-diamine (abbreviation: 2Ph-mmAdtBuDPhA2Anth-02) can be used, for example.

### [Example 2 of Electron-Donating Material]

As the electron-donating material, a phthalocyanine compound, a tetracene derivative, a quinacridone derivative, a rubrene derivative, or the like can be used.

As the electron-donating material, copper(II) phthalocyanine (abbreviation: CuPc), tin(II) phthalocyanine (abbreviation: SnPc), zinc phthalocyanine (abbreviation: ZnPc), tetraphenyldibenzoperiflanthene (abbreviation: DBP), rubrene, or the like can be used.

### <<Structure Example 2 of Layer 114S>>

The layer 114S can have a single-layer structure or a stacked-layer structure, for example. Specifically, the layer 114S can have a bulk heterojunction structure. Alternatively, the layer 114S can have a heterojunction structure.

### [Structure Example of Mixed Material]

A mixed material containing an electron-accepting material and an electron-donating material can be used for the layer 114S, for example (see FIG. 1A). Note that a structure in which such a mixed material containing an electron-accepting material and an electron-donating material is used for the layer 114S can be referred to as a bulk heterojunction structure.

Specifically, a mixed material containing a C₇₀ fullerene and DBP can be used for the layer 114S.

### [Example of Heterojunction Structure]

A layer 114N and a layer 114P can be used for the layer 114S (see FIG. 1). The layer 114N is held between one electrode and the layer 114P, and the layer 114P is held between the layer 114N and the other electrode. For example, the layer 114N is held between the electrode 552S and the layer 114P, and the layer 114P is held between the layer 114N and the electrode 551S.

An n-type semiconductor can be used for the layer 114N. For example, Me-PTCDI can be used for the layer 114N.

A p-type semiconductor can be used for the layer 114P. For example, rubrene can be used for the layer 114P.

Note that the photoelectric conversion device 550S in which the layer 114P is in contact with the layer 114N can be referred to as a pn-junction photodiode.

### <<Structure Example of Layer 112>>

For example, a material having a hole-transport property can be used for the layer 112. The layer 112 can be referred to as a hole-transport layer.

### [Material Having Hole-Transport Property]

A material having a hole mobility higher than or equal to 1×10⁻⁶ cm²/Vs can be suitably used as the material having a hole-transport property.

As the hole-transport material, an amine compound or an organic compound having a π-electron rich heteroaromatic ring skeleton can be used, for example. Specifically, a compound having an aromatic amine skeleton, a compound having a carbazole skeleton, a compound having a thiophene skeleton, a compound having a furan skeleton, or the like can be used. The compound having an aromatic amine skeleton and the compound having a carbazole skeleton are particularly preferable because these compounds are highly reliable and have high hole-transport properties to contribute to a reduction in driving voltage.

As the compound having an aromatic amine skeleton, 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), or the like can be used, for example.

As the compound having a carbazole skeleton, 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), or the like can be used, for example.

As the compound having a thiophene skeleton, 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), or the like can be used, for example.

As the compound having a furan skeleton, 4,4′,4″-(benzene-1,3,5-triyl)tri(dibenzofuran) (abbreviation: DBF3P-II), 4-{3-[3-(9-phenyl-9H-fluoren-9-yl)phenyl]phenyl}dibenzofuran (abbreviation: mmDBFFLBi-II), or the like can be used, for example.

### <<Structure Example of Layer 113>>

A material having an electron-transport property, a material having an anthracene skeleton, or a mixed material can be used for the layer 113, for example. The layer 113 can be referred to as an electron-transport layer.

### [Material Having Electron-Transport Property]

For example, a metal complex or an organic compound having a π-electron deficient heteroaromatic ring skeleton can be used as the material having an electron-transport property.

As a metal complex, bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl)phenolato]zinc(II) (abbreviation: ZnBTZ), or the like can be used, for example.

As an organic compound having a π-electron deficient heteroaromatic ring skeleton, a heterocyclic compound having a polyazole skeleton, a heterocyclic compound having a diazine skeleton, a heterocyclic compound having a pyridine skeleton, a heterocyclic compound having a triazine skeleton, or the like can be used, for example. In particular, the heterocyclic compound having a diazine skeleton or the heterocyclic compound having a pyridine skeleton has favorable reliability and thus are preferable. In addition, the heterocyclic compound having a diazine (pyrimidine or pyrazine) skeleton has a high electron-transport property to contribute to a reduction in driving voltage.

As a heterocyclic compound having a polyazole skeleton, 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or the like can be used, for example.

As a heterocyclic compound having a diazine skeleton, 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,8-bis[3-(dibenzothiophen-4-yl)phenyl]benzo[h]quinazoline (abbreviation: 4,8mDBtP2Bqn), or the like can be used, for example.

As a heterocyclic compound having a pyridine skeleton, 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), or the like can be used, for example.

As a heterocyclic compound having a triazine skeleton, 2-[3′-(9,9-dimethyl-9H-fluoren-2-yl)-1,1′-biphenyl-3-yl]-4,6-diphenyl-1,3,5-triazine (abbreviation: mFBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), or the like can be used, for example.

### [Material Having Anthracene Skeleton]

An organic compound having an anthracene skeleton can be used for the layer 113. In particular, an organic compound having both an anthracene skeleton and a heterocyclic skeleton can be suitably used.

For example, an organic compound having both an anthracene skeleton and a nitrogen-containing five-membered ring skeleton can be used. Alternatively, an organic compound having both an anthracene skeleton and a nitrogen-containing five-membered ring skeleton where two heteroatoms are included in a ring can be used. Specifically, a pyrazole ring, an imidazole ring, an oxazole ring, a thiazole ring, or the like can be favorably used as the heterocyclic skeleton.

For example, an organic compound having both an anthracene skeleton and a nitrogen-containing six-membered ring skeleton can be used. Alternatively, an organic compound having both an anthracene skeleton and a nitrogen-containing six-membered ring skeleton where two heteroatoms are included in a ring can be used. Specifically, a pyrazine ring, a pyrimidine ring, a pyridazine ring, or the like can be favorably used as the heterocyclic skeleton.

### [Structure Example of Mixed Material]

A material in which a plurality of kinds of substances are mixed can be used for the layer 113. Specifically, a mixed material that contains a substance having an electron-transport property and any of an alkali metal, an alkali metal compound, and an alkali metal complex can be used for the layer 113.

The concentration of the alkali metal, the alkali metal compound, or the alkali metal complex preferably differs in the thickness direction of the layer 113 (including the case where the concentration is 0).

For example, a metal complex having an 8-hydroxyquinolinato structure can be used. A methyl-substituted product of the metal complex having an 8-hydroxyquinolinato structure (e.g., a 2-methyl-substituted product or a 5-methyl-substituted product) or the like can also be used.

As the metal complex having an 8-hydroxyquinolinato structure, 8-hydroxyquinolinato-lithium (abbreviation: Liq), 8-hydroxyquinolinato-sodium (abbreviation: Naq), or the like can be used. In particular, a complex of a monovalent metal ion, especially a complex of lithium is preferable, and Liq is further preferable.

Note that this embodiment can be combined with any of the other embodiments in this specification as appropriate.

### Embodiment 3

In this embodiment, a structure of the photoelectric conversion device 550S of one embodiment of the present invention will be described with reference to FIG. 1.

### <Structure Example of Photoelectric Conversion Device 550S>

The photoelectric conversion device 550S described in this embodiment includes the electrode 551S, the electrode 552S, the unit 103S, and a layer 104. The electrode 552S overlaps with the electrode 551S, and the unit 103S is held between the electrode 551S and the electrode 552S. The layer 104 is held between the electrode 551S and the unit 103S. For example, the structure described in Embodiment 2 can be used for the unit 103S.

### <Structure Example of Electrode 551S>

For example, a conductive material can be used for the electrode 551S. Specifically, a single layer or a stacked layer of a metal, an alloy, or a film containing a conductive compound can be used for the electrode 551S.

For example, a film that efficiently reflects light can be used for the electrode 551S. Specifically, an alloy containing silver, copper, and the like, an alloy containing silver, palladium, and the like, or a metal film of aluminum or the like can be used for the electrode 551S.

Alternatively, for example, a metal film that transmits part of light and reflects the other part of the light can be used as the electrode 551S. Thus, a microcavity structure (microcavity) can be provided in the photoelectric conversion device 550S.

A film having a transmitting property with respect to visible light can be used for the electrode 551S, for example. Specifically, a single layer or a stacked layer of a metal film, an alloy film, a conductive oxide film, or the like that is thin enough to transmit light can be used for the electrode 551S.

In particular, a material having a work function higher than or equal to 4.0 eV can be suitably used for the electrode 551S.

For example, a conductive oxide containing indium can be used. Specifically, indium oxide, indium oxide-tin oxide (abbreviation: ITO), indium oxide-tin oxide containing silicon or silicon oxide (abbreviation: ITSO), indium oxide-zinc oxide, indium oxide containing tungsten oxide and zinc oxide (abbreviation: IWZO), or the like can be used.

Furthermore, for example, a conductive oxide containing zinc can be used. Specifically, zinc oxide, zinc oxide to which gallium is added, zinc oxide to which aluminum is added, or the like can be used.

Furthermore, for example, gold (Au), platinum (Pt), nickel (Ni), tungsten (W), chromium (Cr), molybdenum (Mo), iron (Fe), cobalt (Co), copper (Cu), palladium (Pd), a nitride of a metal material (e.g., titanium nitride), or the like can be used. Alternatively, graphene can be used.

### <<Structure Example 1 of Layer 104>>

For example, a material having a hole-injection property can be used for the layer 104. The layer 104 can be referred to as a hole-injection layer.

For example, a material having a hole mobility lower than or equal to 1×10⁻³ cm/Vs when the square root of the electric field strength [V/cm] is 600 can be used for the layer 104. A film having a resistivity greater than or equal to 1×10⁴ [Ω·cm] and less than or equal to 1×10⁷ [Ω·cm] can be used as the layer 104. The resistivity of the layer 104 is preferably greater than or equal to 5×10⁴ [Ω·cm] and less than or equal to 1×10⁷ [Ω·cm], further preferably greater than or equal to 1×10⁵ [Ω·cm] and less than or equal to 1×10⁷ [Ω·cm].

### <<Structure Example 2 of Layer 104>>

Specifically, a substance having an electron-accepting property can be used for the layer 104. A composite material containing a plurality of kinds of substances can be used for the layer 104.

### [Substance Having Electron-Accepting Property]

An organic compound and an inorganic compound can be used as the substance having an electron-accepting property. The substance having an electron-accepting property can extract electrons from an adjacent hole-transport layer or an adjacent material having a hole-transport property by the application of an electric field.

For example, a compound having an electron-withdrawing group (a halogen group or a cyano group) can be used as the substance having an electron-accepting property. Note that an organic compound having an electron-accepting property is easily evaporated and deposited. As a result, the productivity of the photoelectric conversion device 550S can be increased.

Specifically, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene)malononitrile can be used, for example.

A compound in which electron-withdrawing groups are bonded to a condensed aromatic ring having a plurality of heteroatoms, such as HAT-CN, is particularly preferable because it is thermally stable.

Alternatively, a [3]radialene derivative having an electron-withdrawing group (in particular, a cyano group or a halogen group such as a fluoro group) is preferable because it has a very high electron-accepting property.

Specifically, α,α′,a′″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], or α,α′,α″-1,2,3-cyclopropanetriylidenetris[2,3,4,5,6-pentafluorobenzeneacetonitrile] can be used, for example.

As the substance having an electron-accepting property, molybdenum oxide, vanadium oxide, ruthenium oxide, tungsten oxide, manganese oxide, or the like can be used.

Alternatively, phthalocyanine (abbreviation: H₂Pc), a phthalocyanine-based complex compound such as copper phthalocyanine (CuPc), and compounds having an aromatic amine skeleton such as 4,4′-bis[N-(4-diphenylaminophenyl)-N-phenylamino]biphenyl (abbreviation: DPAB) and N,N′-bis{4-[bis(3-methylphenyl)amino]phenyl}-N,N′-diphenyl-(1,1′-biphenyl)-4,4′-diamine (abbreviation: DNTPD) can be used.

Furthermore, a high molecular compound such as poly(3,4-ethylenedioxythiophene)/poly(styrenesulfonic acid) (PEDOT/PSS) can be used.

### [Structure Example 1 of Composite Material]

For example, a composite material containing a substance having an electron-accepting property and a material having a hole-transport property can be used for the layer 104. Thus, not only a material having a high work function, but also a material having a low work function can be used for the electrode 551S. Alternatively, a material used for the electrode 551S can be selected from a wide range of materials regardless of its work function.

As the material having a hole-transport property in the composite material, for example, a compound having an aromatic amine skeleton, a carbazole derivative, an aromatic hydrocarbon, an aromatic hydrocarbon having a vinyl group, a high molecular compound (such as an oligomer, a dendrimer, or a polymer), or the like can be used. A material having a hole mobility higher than or equal to 1×10⁻⁶ cm²/Vs can be suitably used as the material having a hole-transport property in the composite material.

A substance having a relatively deep HOMO level can be suitably used as the material having a hole-transport property in the composite material. Specifically, the HOMO level is preferably higher than or equal to −5.7 eV and lower than or equal to −5.4 eV.

As the compound having an aromatic amine skeleton, for example, N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), or the like can be used.

As the carbazole derivative, for example, 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthracenyl)phenyl]-9H-carbazole (abbreviation: CzPA), 1,4-bis[4-(N-carbazolyl)phenyl]-2,3,5,6-tetraphenylbenzene, or the like can be used.

As the aromatic hydrocarbon, for example, 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, pentacene, coronene, or the like can be used.

As the aromatic hydrocarbon having a vinyl group, for example, 4,4′-bis(2,2-diphenylvinyl)biphenyl (abbreviation: DPVBi), 9,10-bis[4-(2,2-diphenylvinyl)phenyl]anthracene (abbreviation: DPVPA), or the like can be used.

As the high molecular compound, for example, poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), poly[N,N′-bis(4-butylphenyl)-N,N′-bis(phenyl)benzidine] (abbreviation: Poly-TPD), or the like can be used.

As another example, a substance having any of a carbazole skeleton, a dibenzofuran skeleton, a dibenzothiophene skeleton, and an anthracene skeleton can be favorably used as the material having a hole-transport property in the composite material. Moreover, as the material having a hole-transport property in the composite material, it is possible to use a substance including any of an aromatic amine having a substituent that includes a dibenzofuran ring or a dibenzothiophene ring, an aromatic monoamine that includes a naphthalene ring, and an aromatic monoamine in which a 9-fluorenyl group is bonded to nitrogen of amine through an arylene group. With the use of a substance including an N,N-bis(4-biphenyl)amino group, the reliability of the photoelectric conversion device 550S can be increased.

As these materials, any of the following can be used, for example: N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-diphenyl-4′-(2-naphthyl)-4″-{9-(4-biphenylyl)carbazole)}triphenylamine (abbreviation: YGTBiβNB), N-[4-(9-phenyl-9Hcarbazol-3-yl)phenyl]-N-[4-(1-naphthyl)phenyl]-9,9′-spirobi[9H-fluoren]-2-amine (abbreviation: PCBNBSF), N,N-bis(4-biphenylyl)-9,9′-spirobi[9H-fluoren]-2-amine (abbreviation: BBASF), N,N-bis(1,1′-biphenyl-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(dibenzofuran-4-yl)-9,9-dimethyl-9H-fluoren-2-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]spiro-9,9′-bifluoren-2-amine (abbreviation: PCBASF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 9-dimethyl-N-[4-(9-phenyl-9H-carbazol-3-yl)phenyl]-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, N,N-bis(9,9-dimethyl-9H-fluoren-2-yl)-9,9′-spirobi-9H-fluoren-1-amine, and the like.

### [Structure Example 2 of Composite Material]

For example, a composite material containing a substance having an electron-accepting property, a material having a hole-transport property, and a fluoride of an alkali metal or a fluoride of an alkaline earth metal can be used as the material having a hole-injection property. In particular, a composite material in which the proportion of fluorine atoms is higher than or equal to 20% can be suitably used. Thus, the refractive index of the layer 104 can be reduced.

Note that this embodiment can be combined with any of the other embodiments in this specification as appropriate.

### Embodiment 4

In this embodiment, a structure of the photoelectric conversion device 550S of one embodiment of the present invention will be described with reference to FIG. 1.

### <Structure Example of Photoelectric Conversion Device 550S>

The photoelectric conversion device 550S described in this embodiment includes the electrode 551S, the electrode 552S, the unit 103S, and a layer 105. The electrode 552S includes a region overlapping with the electrode 551S, and the unit 103S includes a region held between the electrode 551S and the electrode 552S. The layer 105 includes a region held between the unit 103S and the electrode 552S. For example, the structure described in Embodiment 2 can be used for the unit 103S.

### <Structure Example of Electrode 552S>

A conductive material can be used for the electrode 552S, for example. Specifically, a single layer or a stacked layer of a metal, an alloy, or a material containing a conductive compound can be used for the electrode 552S.

For example, the material that can be used for the electrode 551S described in Embodiment 3 can be used for the electrode 552S. In particular, a material having a lower work function than the electrode 551S can be favorably used for the electrode 552S.

For example, an element belonging to Group 1 of the periodic table, an element belonging to Group 2 of the periodic table, a rare earth metal, or an alloy containing any of these elements can be used for the electrode 552S.

Specifically, lithium (Li), cesium (Cs), or the like; magnesium (Mg), calcium (Ca), strontium (Sr), or the like; europium (Eu), ytterbium (Yb), or the like; or an alloy containing any of these such as an alloy of magnesium and silver or an alloy of aluminum and lithium can be used for the electrode 552S.

### <<Structure Example of Layer 105>>

A material having an electron-injection property can be used for the layer 105, for example. The layer 105 can be referred to as an electron-injection layer.

Specifically, a substance having a donor property can be used for the layer 105. Alternatively, a material in which a substance having a donor property and a material having an electron-transport property are combined can be used for the layer 105. Alternatively, electride can be used for the layer 105. This can facilitate injection of electrons from the electrode 552S, for example. Alternatively, besides a material having a low work function, a material having a high work function can also be used for the electrode 552S. Alternatively, a material used for the electrode 552S can be selected from a wide range of materials regardless of its work function. Specifically, Al, Ag, ITO, indium oxide-tin oxide containing silicon or silicon oxide, or the like can be used for the electrode 552.S

### [Substance Having Donor Property]

For example, an alkali metal, an alkaline earth metal, a rare earth metal, or a compound thereof (an oxide, a halide, a carbonate, or the like) can be used as the substance having a donor property. Alternatively, an organic compound such as tetrathianaphthacene (abbreviation: TTN), nickelocene, or decamethylnickelocene can be used as the substance having a donor property.

As an alkali metal compound (including an oxide, a halide, and a carbonate), lithium oxide, lithium fluoride (LiF), cesium fluoride (CsF), lithium carbonate, cesium carbonate, 8-hydroxyquinolinato-lithium (abbreviation: Liq), or the like can be used.

As an alkaline earth metal compound (including an oxide, a halide, and a carbonate), calcium fluoride (CaF₂) or the like can be used.

### [Structure Example 1 of Composite Material]

A material in which a plurality of kinds of substances are combined can be used as the material having an electron-injection property. For example, a substance having a donor property and a material having an electron-transport property can be used for the composite material.

### [Material Having Electron-Transport Property]

For example, a metal complex or an organic compound having a π-electron deficient heteroaromatic ring skeleton can be used as the material having an electron-transport property.

For example, a material having an electron-transport property usable for the layer 113 described in Embodiment 2 can be used for the composite material. In particular, a material having an electron mobility higher than or equal to 1×10⁻⁷ cm²/Vs and lower than or equal to 5×10⁻⁵ cm²/Vs in a condition where the square root of the electric field strength [V/cm] is 600 can be favorably used as the material having an electron-transport property.

### [Structure Example 2 of Composite Material]

A material including a fluoride of an alkali metal in a microcrystalline state and a material having an electron-transport property can be used for the composite material. Alternatively, a material including a fluoride of an alkaline earth metal in a microcrystalline state and a material having an electron-transport property can be used for the composite material. In particular, a composite material containing a fluoride of an alkali metal or a fluoride of an alkaline earth metal at higher than or equal to 50 wt % can be suitably used. Alternatively, a composite material including an organic compound having a bipyridine skeleton can be suitably used. In that case, the refractive index of the layer 105 can be reduced.

### [Structure Example 3 of Composite Material]

For example, a composite material containing a first organic compound having an unshared electron pair and a first metal can be used for the layer 105. The sum of the number of electrons of the first organic compound and the number of electrons of the first metal is preferably an odd number. The molar ratio of the first metal to 1 mol of the first organic compound is preferably greater than or equal to 0.1 and less than or equal to 10, further preferably greater than or equal to 0.2 and less than or equal to 2, and still further preferably greater than or equal to 0.2 and less than or equal to 0.8.

Accordingly, the first organic compound having an unshared electron pair interacts with the first metal and thus can form a singly occupied molecular orbital (SOMO). Furthermore, in the case where electrons are injected from the electrode 552S into the layer 105, a barrier therebetween can be lowered. The first metal has a low reactivity with water and oxygen; thus, the moisture resistance of the photoelectric conversion device 550S can be improved.

For the layer 105, a composite material that allows the spin density measured by an electron spin resonance (ESR) method to be preferably higher than or equal to 1×10¹⁶ spins/cm³, further preferably higher than or equal to 5×10¹⁶ spins/cm³, still further preferably higher than or equal to 1×10¹⁷ spins/cm³ can be used.

### [Organic Compound Having Unshared Electron Pair]

For example, a material having an electron-transport property can be used for the organic compound having an unshared electron pair. For example, a compound having an electron deficient heteroaromatic ring can be used. Specifically, a compound having at least one of a pyridine ring, a diazine ring (a pyrimidine ring, a pyrazine ring, and a pyridazine ring), and a triazine ring can be used.

Note that the lowest unoccupied molecular orbital (LUMO) level of the organic compound having an unshared electron pair is preferably greater than or equal to −3.6 eV and less than or equal to −2.3 eV. In general, the HOMO level and the LUMO level of an organic compound can be estimated by CV (cyclic voltammetry), photoelectron spectroscopy, optical absorption spectroscopy, inverse photoelectron spectroscopy, or the like.

For example, 4,7-diphenyl-1,10-phenanthroline (abbreviation: BPhen), 2,9-di(naphthalen-2-yl)-4,7-diphenyl-1,10-phenanthroline (abbreviation: NBPhen), diquinoxalino[2,3-a:2′,3′-c]phenazine (abbreviation: HATNA), 2,4,6-tris[3′-(pyridin-3-yl)biphenyl-3-yl]-1,3,5-triazine (abbreviation: TmPPPyTz), or the like can be used for the organic compound having an unshared electron pair. Note that NBPhen has a higher glass transition temperature (Tg) than BPhen and thus has high heat resistance.

Alternatively, for example, copper phthalocyanine can be used for the organic compound having an unshared electron pair. The number of electrons of the copper phthalocyanine is an odd number.

### [First Metal]

For example, when the number of electrons of the first organic compound having an unshared electron pair is an even number, a composite material of a metal that belongs to an odd-numbered group in the periodic table and the first organic compound can be used for the layer 105.

For example, manganese (Mn), which is a metal belonging to Group 7, cobalt (Co), which is a metal belonging to Group 9, copper (Cu), silver (Ag), and gold (Au), which are metals belonging to Group 11, aluminum (Al) and indium (In), which are metals belonging to Group 13 are odd-numbered groups in the periodic table. Note that elements belonging to Group 11 have a lower melting point than elements belonging to Group 7 or Group 9 and thus are suitable for vacuum evaporation. In particular, Ag is preferable because of its low melting point.

The use of Ag for the electrode 552S and the layer 105 can increase the adhesion between the layer 105 and the electrode 552S.

When the number of electrons of the first organic compound having an unshared electron pair is an odd number, a composite material of the first metal that belongs to an even-numbered group in the periodic table and the first organic compound can be used for the layer 105. For example, iron (Fe), which is a metal belonging to Group 8, is an element belonging to an even-numbered group in the periodic table.

### [Electride]

For example, a substance obtained by adding electrons at high concentration to an oxide where calcium and aluminum are mixed, or the like can be used as the material having an electron-injection property.

Note that this embodiment can be combined with any of the other embodiments in this specification as appropriate.

### Embodiment 5

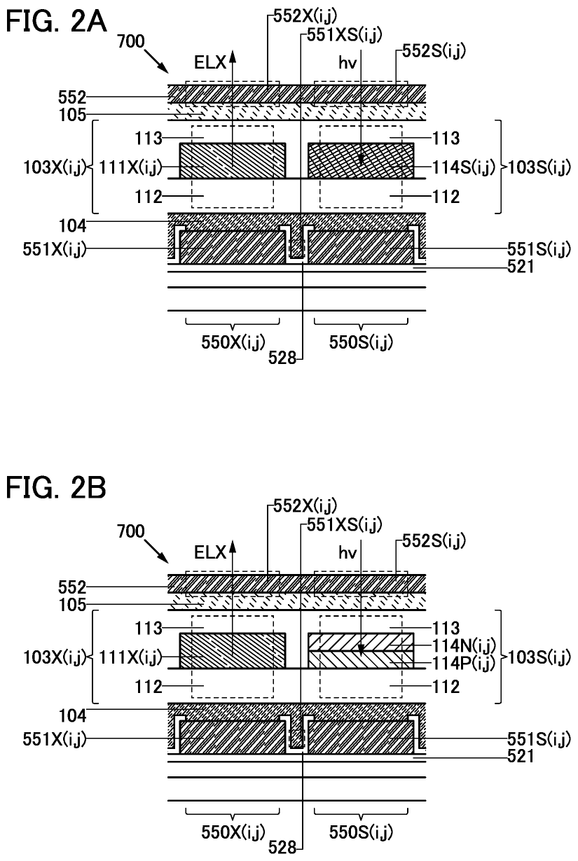

In this embodiment, a structure of a display device 700 of one embodiment of the present invention will be described with reference to FIG. 2A and FIG. 2B.

FIG. 2A is a cross-sectional view illustrating a structure of the display device 700 of one embodiment of the present invention, and FIG. 2B is a cross-sectional view illustrating a structure of the display device 700 of one embodiment of the present invention different from that in FIG. 2A.

### <Structure Example 1 of Display Device 700>

The display device 700 described in this embodiment includes a light-emitting device 550X(i,j) and a photoelectric conversion device 550S(i,j) (see FIG. 2A). The light-emitting device 550X(i,j) is adjacent to the photoelectric conversion device 550S(i,j).

The display device 700 further includes an insulating film 521, and the photoelectric conversion device 550S(i,j) and the light-emitting device 550X(i,j) are formed over the insulating film 521.

### <<Structure Example 1 of Photoelectric Conversion Device 550S(i,j)>>

The photoelectric conversion device 550S(i,j) includes an electrode 551S(i,j), an electrode 552S(i,j), and a unit 103S(i,j). Furthermore, the photoelectric conversion device 550S(i,j) includes the layer 104 and the layer 105.

For example, the photoelectric conversion device described in any of Embodiment 2 to Embodiment 4 can be used as the photoelectric conversion device 550S(i,j). Specifically, the structure usable for the electrode 551S can be used for the electrode 551S(i,j). The structure usable for the unit 103S can be used for the unit 103S(i,j). The structure described in Embodiment 3 can be used for the layer 104, and the structure described in Embodiment 5 can be used for the layer 105.

### <<Structure Example 1 of Light-Emitting Device 550X(i,j)>>

The light-emitting device 550X(i,j) includes an electrode 551X(i,j), an electrode 552X(i,j), and a unit 103X(i,j) (see FIG. 2A). The electrode 552X(i,j) overlaps with the electrode 551X(i,j), and the unit 103X(i,j) is held between the electrode 551X(i,j) and the electrode 552X(i, j).

The electrode 551X(i,j) is adjacent to the electrode 551S(i,j), and a gap 551XS(i,j) is provided between the electrode 551X(i,j) and the electrode 551X(i,j).

For example, a material usable for the electrode 551S(i,j) can be used for the electrode 551X(i,j).

### <<Structure Example 1 of Unit 103X(i,j)>>

The unit 103X(i,j) has a single-layer structure or a stacked-layer structure. For example, the unit 103X(i,j) includes a layer 111X(i,j), the layer 112, and the layer 113 (see FIG. 2A). The layer 111X(i,j) is held between the layer 112 and the layer 113, the layer 112 is held between the electrode 551X(i, j) and the layer 111X(i, j), and the layer 113 is held between the electrode 552X(i,j) and the layer 111X(i,j).

For example, a layer selected from functional layers such as a light-emitting layer, a hole-transport layer, an electron-transport layer, and a carrier-blocking layer can be used in the unit 103X(i, j). Moreover, a layer selected from functional layers such as a hole-injection layer, an electron-injection layer, an exciton-blocking layer, and a charge-generation layer can be used in the unit 103X(i, j).

### <<Structure Example 2 of Light-Emitting Device 550X(i,j)>>

The light-emitting device 550X(i,j) includes the layer 104 and the layer 105. The layer 104 is held between the electrode 551X(i,j) and the unit 103X(i,j), and the layer 105 is held between the unit 103X(i,j) and the electrode 552X(i,j).

Note that some of the components of the photoelectric conversion device 550S(i,j) can be used as some of the components of the light-emitting device 550X(i,j). Thus, some of the components can be used in common. Alternatively, the manufacturing process can be simplified.

### <Structure Example 2 of Display Device 700>

The display device 700 described in this embodiment includes an insulating film 528 (see FIG. 2A).

### <<Structure Example of Insulating Film 528>>

The insulating film 528 has openings; one opening overlaps with the electrode 551S(i,j) and the other opening overlaps with the electrode 551X(i,j).

### <Structure Example 3 of Display Device 700>

The display device 700 described in this embodiment includes the layer 111X(i,j) (see FIG. 2A or FIG. 2B).

### <<Structure Example 1 of Layer 111X(i,j)>>

A light-emitting material or a light-emitting material and a host material can be used for the layer 111X(i,j), for example. The layer 111X(i,j) can be referred to as a light-emitting layer. The layer 111X(i, j) is preferably provided in a region where holes and electrons are recombined. In that case, energy generated by recombination of carriers can be efficiently converted into light and emitted. Furthermore, the layer 111X(i,j) is preferably provided apart from a metal used for the electrode or the like. In that case, a quenching phenomenon caused by the metal used for the electrode or the like can be inhibited.

For example, a light-emitting device that emits blue light, a light-emitting device that emits green light, and a light-emitting device that emits red light can be provided in the display device 700. Alternatively, a light-emitting device that emits white light, a light-emitting device that emits yellow light, or a light-emitting device that emits infrared rays can be provided in the display device 700.

<<Structure Example 2 of Layer 111X(i, j)>>

For example, a fluorescent substance, a phosphorescent substance, or a substance exhibiting thermally activated delayed fluorescence (TADF) (also referred to as a TADF material) can be used as the light-emitting material. Thus, energy generated by recombination of carriers can be released as light ELX from the light-emitting material (see FIG. 2A or FIG. 2B).

### [Fluorescent Substance]

A fluorescent substance can be used for the layer 111X(i,j). For example, the following fluorescent substances can be used for the layer 111X(i,j). Note that without being limited to the following ones, a variety of known fluorescent substances can be used for the layer 111X(i,j).

Specifically, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-(pyrene-1,6-diyl)bis[(6,N-diphenylbenzo[b]naphtho[1,2-d]furan)-8-amine](abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02).

Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

### [Phosphorescent Substance]

A phosphorescent substance can be used for the layer 111X(i, j). For example, the following phosphorescent substances can be used for the layer 111X(i,j). Note that without being limited to the following ones, a variety of known phosphorescent substances can be used for the layer 111X(i,j).

For the layer 111X(i, j), it is possible to use, for example, an organometallic iridium complex having a 4H-triazole skeleton, an organometallic iridium complex having a 1H-triazole skeleton, an organometallic iridium complex having an imidazole skeleton, an organometallic iridium complex having a phenylpyridine derivative with an electron-withdrawing group as a ligand, an organometallic iridium complex having a pyrimidine skeleton, an organometallic iridium complex having a pyrazine skeleton, an organometallic iridium complex having a pyridine skeleton, a rare earth metal complex, and a platinum complex.

### [Phosphorescent Substance (Blue)]

As an organometallic iridium complex having a 4H-triazole skeleton or the like, tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), or the like can be used.

As an organometallic iridium complex having a 1H-triazole skeleton or the like, tris[3-methyl-1-(2-methylphenyl)-5-phenyl-1H-1,2,4-triazolato]iridium(III) (abbreviation: [Ir(Mptz1-mp)₃]), tris(1-methyl-5-phenyl-3-propyl-1H-1,2,4-triazolato]iridium(III) (abbreviation: [Ir(Prptz1-Me)₃]), or the like can be used.

As an organometallic iridium complex having an imidazole skeleton or the like, fac-tris[1-(2,6-diisopropylphenyl)-2-phenyl-1H-imidazole]iridium(III) (abbreviation: [Ir(iPrpmi)₃]), tris[3-(2,6-dimethylphenyl)-7-methylimidazo[1,2-j]phenanthridinato]iridium(III) (abbreviation: [Ir(dmpimpt-Me)₃]), or the like can be used.

As an organometallic iridium complex having a phenylpyridine derivative with an electron-withdrawing group as a ligand, or the like, bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), bis[2-(4′,6′-difluorophenyl)pyridinato-N,C^{2′}]iridium(III) acetylacetonate (abbreviation: FIracac), or the like can be used.

Note that these are compounds exhibiting blue phosphorescence and are compounds having an emission wavelength peak at 440 nm to 520 nm.

### [Phosphorescent Substance (Green)]

As an organometallic iridium complex having a pyrimidine skeleton or the like, it is possible to use, for example, tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]).

As an organometallic iridium complex having a pyrazine skeleton or the like, (acetylacetonato)bis(3,5-dimethyl-2-phenylpyrazinato)iridium(III) (abbreviation: [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), or the like can be used.

As an organometallic iridium complex having a pyridine skeleton or the like, it is possible to use, for example, tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d3-methyl-(2-pyridinyl-κN)benzofuro[2,3-b]pyridine-κC]bis[2-(5-d3-methyl-2-pyridinyl-κN2)phenyl-κC]iridium(III) (abbreviation: [Ir(5mppy-d3)₂(mbfpypy-d3)]), or [2-d3-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d3)]).

An example of a rare earth metal complex is tris(acetylacetonato) (monophenanthroline)terbium(III) (abbreviation: [Tb(acac)₃(Phen)]).

Note that these are compounds mainly exhibiting green phosphorescence and have an emission wavelength peak at 500 nm to 600 nm. An organometallic iridium complex having a pyrimidine skeleton excels particularly in reliability or emission efficiency.

### [Phosphorescent Substance (Red)]

As an organometallic iridium complex having a pyrimidine skeleton or the like, (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), bis[4,6-di(naphthalen-1-yl)pyrimidinato] (dipivaloylmethanato)iridium(III) (abbreviation: [Ir(d1npm)₂(dpm)]), or the like can be used.

As an organometallic iridium complex having a pyrazine skeleton or the like, (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), (acetylacetonato)bis[2,3-bis(4-fluorophenyl)quinoxalinato]iridium(III) (abbreviation: [Ir(Fdpq)₂(acac)]), or the like can be used.

As an organometallic iridium complex having a pyridine skeleton or the like, tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), or the like can be used.

As a rare earth metal complex or the like, tris(1,3-diphenyl-1,3-propanedionato)(monophenanthroline)europium(III) (abbreviation: [Eu(DBM)₃(Phen)]), tris[1-(2-thenoyl)-3,3,3-trifluoroacetonato] (monophenanthroline)europium(III) (abbreviation: [Eu(TTA)₃(Phen)]), or the like can be used.

As a platinum complex or the like, 2,3,7,8,12,13,17,18-octaethyl-21H,23H-porphyrin platinum(II) (abbreviation: PtOEP) or the like can be used.

Note that these are compounds exhibiting red phosphorescence and have an emission peak at 600 nm to 700 nm. Furthermore, from the organometallic iridium complex having a pyrazine skeleton, red light emission with chromaticity favorably used for display devices can be obtained.

### [Substance Exhibiting Thermally Activated Delayed Fluorescence (TADF)]

A TADF material can be used for the layer 111X(i,j). For example, any of the TADF materials given below can be used as the light-emitting material. Note that without being limited thereto, any of a variety of known TADF materials can be used as the light-emitting material.

In the TADF material, the difference between the S1 level and the T1 level is small, and reverse intersystem crossing (upconversion) from the triplet excited state into the singlet excited state can be achieved by a little thermal energy. Thus, the singlet excited state can be efficiently generated from the triplet excited state. In addition, the triplet excitation energy can be converted into luminescence.

An exciplex whose excited state is formed of two kinds of substances has an extremely small difference between the S1 level and the T1 level and functions as a TADF material capable of converting triplet excitation energy into singlet excitation energy.

A phosphorescent spectrum observed at a low temperature (e.g., 77 K to 10 K), for example, is used for an index of the T1 level. For example, when the level of energy with a wavelength of the line obtained by extrapolating a tangent to the fluorescent spectrum at a tail on the short wavelength side is the S1 level and the level of energy with a wavelength of the line obtained by extrapolating a tangent to the phosphorescent spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than that of the TADF material. In addition, the T1 level of the host material is preferably higher than that of the TADF material.

Examples of the TADF material include a fullerene, a derivative thereof, an acridine, a derivative thereof, and an eosin derivative. Furthermore, porphyrin containing a metal such as magnesium (Mg), zinc (Zn), cadmium (Cd), tin (Sn), platinum (Pt), indium (In), or palladium (Pd) can be also used for the TADF material.

Specifically, any of the following materials whose structural formulae are shown below can be used: a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), an octaethylporphyrin-platinum chloride complex (PtCl₂OEP), and the like.

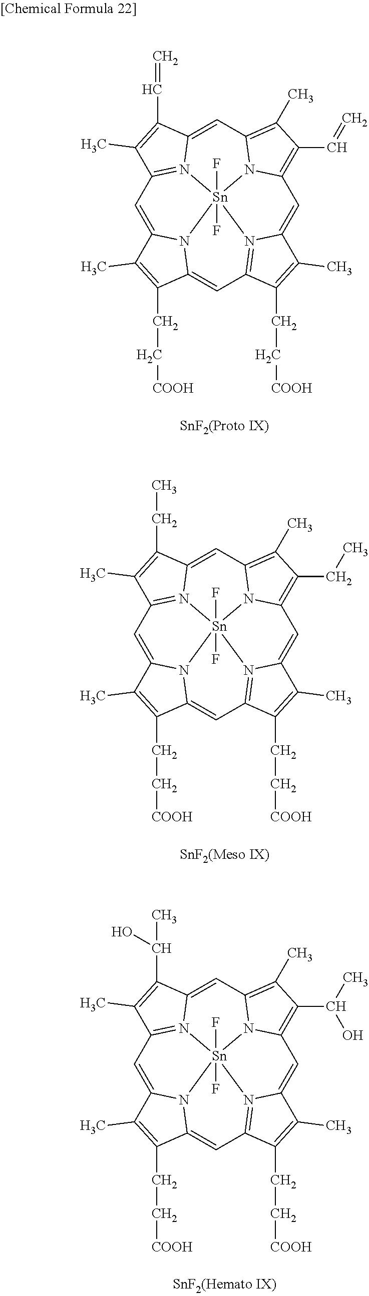

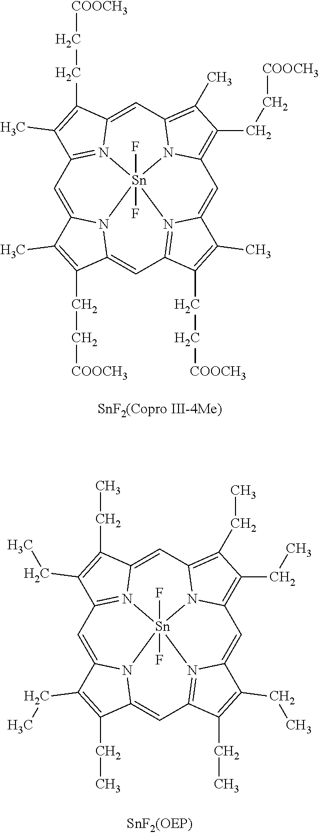

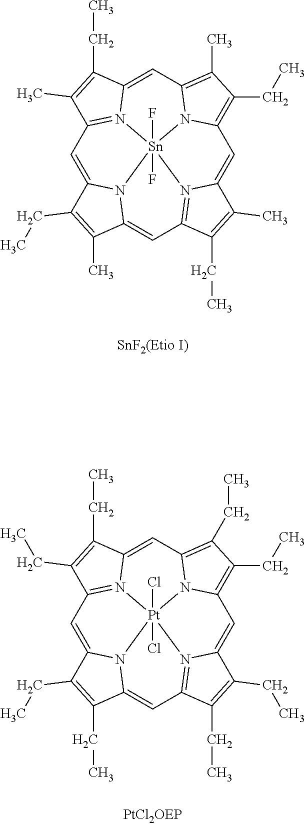

Furthermore, a heterocyclic compound including one or both of a π-electron rich heteroaromatic ring and a π-electron deficient heteroaromatic ring can be used, for example, for the TADF material.

Specifically, any of the following materials whose structural formulae are shown below can be used: 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), and the like.

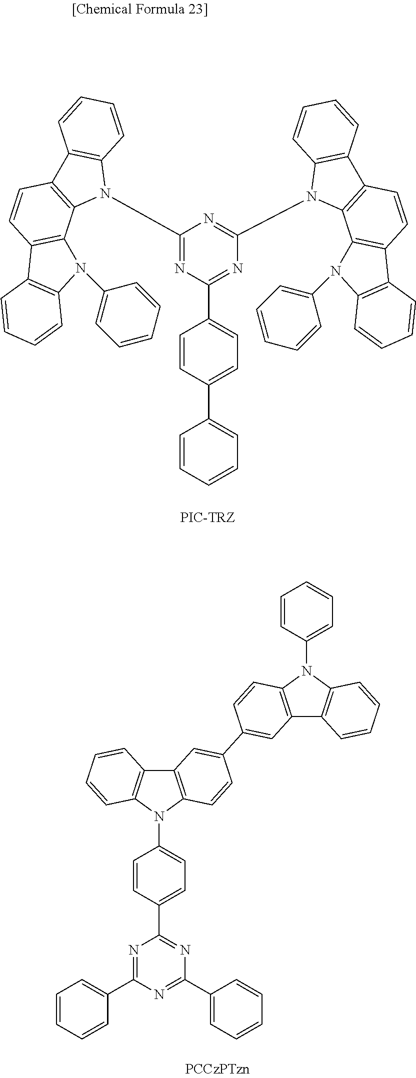 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

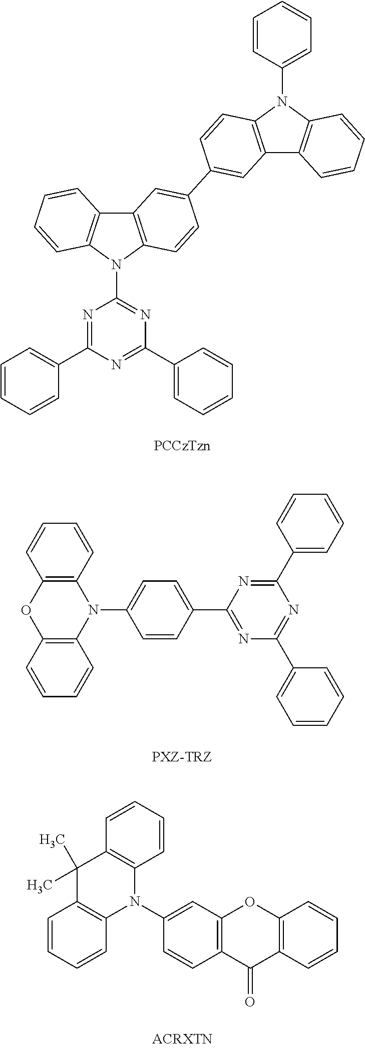 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(N4c5ccccc5Oc5ccccc54)cc3)n2)cc1 ; CC1(C)c2ccccc2N(c2ccc3c(=O)c4ccccc4oc3c2)c2ccccc21]

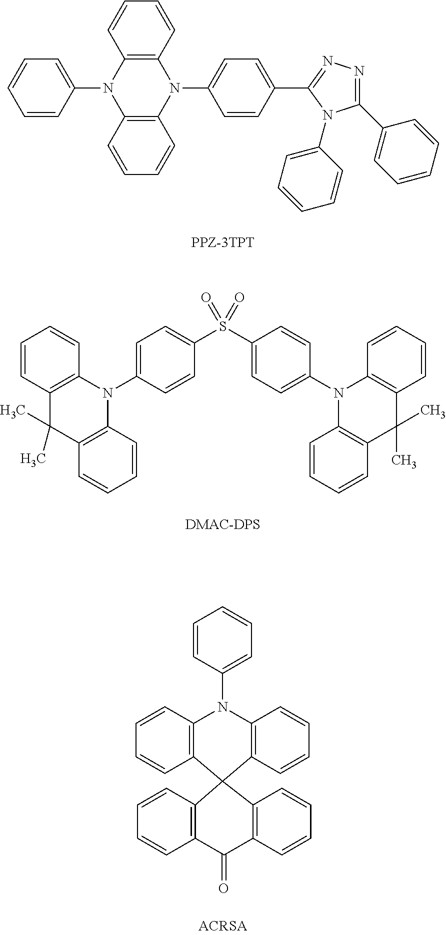 [SMILES: c1ccc(-c2nnc(-c3ccc(N4c5ccccc5N(c5ccccc5)c5ccccc54)cc3)n2-c2ccccc2)cc1 ; CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Such a heterocyclic compound is preferable because of having excellent electron-transport and hole-transport properties owing to a π-electron rich heteroaromatic ring and a π-electron deficient heteroaromatic ring. Among skeletons having the π-electron deficient heteroaromatic ring, in particular, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high electron-accepting properties and reliability.

Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. Note that preferable examples of the furan skeleton and the thiophene skeleton are a dibenzofuran skeleton and a dibenzothiophene skeleton, respectively. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton, for example, are particularly preferable.

Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used.

As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a nitrile group or a cyano group such as benzonitrile or cyanobenzene, a carbonyl skeleton such as benzophenone, a phosphine oxide skeleton, a sulfone skeleton, or the like can be used.

As described above, a π-electron deficient skeleton and a π-electron rich skeleton can be used instead of at least one of the π-electron deficient heteroaromatic ring and the π-electron rich heteroaromatic ring.

### <<Structure Example 3 of Layer 111X(i,j)>>

A material having a carrier-transport property can be used as the host material. For example, a material having a hole-transport property, a material having an electron-transport property, a substance exhibiting thermally activated delayed fluorescence TADF, a material having an anthracene skeleton, or a mixed material can be used as the host material. A material having a wider band gap than the light-emitting material contained in the layer 111X(i, j) is preferably used as the host material. Thus, energy transfer from excitons generated in the layer 111X(i, j) to the host material can be inhibited.

### [Material Having Hole-Transport Property]

A material having a hole mobility higher than or equal to 1×10⁻⁶ cm²/Vs can be suitably used as the material having a hole-transport property.

For example, a material having a hole-transport property usable for the layer 112 can be used for the layer 111X(i,j). Specifically, a material having a hole-transport property usable for the hole-transport layer can be used for the layer 111X(i, j).

### [Material Having Electron-Transport Property]

For example, a metal complex or an organic compound having a π-electron deficient heteroaromatic ring skeleton can be used as the material having an electron-transport property.

For example, a material having an electron-transport property usable for the layer 113 described in Embodiment 2 can be used for the layer 111X.

### [Material Having Anthracene Skeleton]

An organic compound having an anthracene skeleton can be used as the host material. In particular, when a fluorescent substance is used as the light-emitting substance, an organic compound having an anthracene skeleton is suitable. In that case, a light-emitting device with high emission efficiency and high durability can be obtained.

As the organic compound having an anthracene skeleton, for example, an organic compound having a diphenylanthracene skeleton, in particular, a 9,10-diphenylanthracene skeleton is chemically stable and thus is preferable. The host material preferably has a carbazole skeleton, in which case the hole-injection and hole-transport properties are improved. In particular, the host material preferably has a dibenzocarbazole skeleton, in which case the HOMO level thereof is shallower than that of carbazole by approximately 0.1 eV, so that holes enter the host material easily, the hole-transport property is improved, and the heat resistance is increased. Note that in terms of the hole-injection and hole-transport properties, a benzofluorene skeleton or a dibenzofluorene skeleton may be used instead of a carbazole skeleton.

Thus, a substance having both a 9,10-diphenylanthracene skeleton and a carbazole skeleton, a substance having both a 9,10-diphenylanthracene skeleton and a benzocarbazole skeleton, or a substance having both a 9,10-diphenylanthracene skeleton and a dibenzocarbazole skeleton is preferable as the host material.

For example, it is possible to use 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), 9-(1-naphthyl)-10-[4-(2-naphthyl)phenyl]anthracene (abbreviation: αN-βNPAnth), 9-phenyl-3-[4-(10-phenyl-9-anthryl)phenyl]-9H-carbazole (abbreviation: PCzPA), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 3-[4-(1-naphthyl)-phenyl]-9-phenyl-9H-carbazole (abbreviation: PCPN).

In particular, CzPA, cgDBCzPA, 2mBnfPPA, and PCzPA have excellent characteristics.

### [Substance Exhibiting Thermally Activated Delayed Fluorescence (TADF)]

A TADF material can be used as the host material. When the TADF material is used as the host material, triplet excitation energy generated in the TADF material can be converted into singlet excitation energy by reverse intersystem crossing. Moreover, excitation energy can be transferred to the light-emitting substance. In other words, the TADF material functions as an energy donor, and the light-emitting substance functions as an energy acceptor. Thus, the emission efficiency of the light-emitting device can be increased.

This is very effective in the case where the light-emitting substance is a fluorescent substance. In that case, the S1 level of the TADF material is preferably higher than that of the fluorescent substance in order that high emission efficiency can be achieved. Furthermore, the T1 level of the TADF material is preferably higher than the S1 level of the fluorescent substance. Thus, the T1 level of the TADF material is preferably higher than that of the fluorescent substance.

It is also preferable to use a TADF material that emits light whose wavelength overlaps with the wavelength on a lowest-energy-side absorption band of the fluorescent substance. This case is preferable because excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, for example, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination.

Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring.

Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

For example, the TADF material usable as the light-emitting material can be used as the host material.

### [Structure Example 1 of Mixed Material]

A material in which a plurality of kinds of substances are mixed can be used as the host material. For example, a material having an electron-transport property and a material having a hole-transport property can be used for the mixed material. The weight ratio of the material having a hole-transport property to the material having an electron-transport property in the mixed material, i.e., the value of the ratio of the material having a hole-transport property to the material having an electron-transport property, is greater than or equal to 1/19 and less than or equal to 19. Accordingly, the carrier-transport property of the layer 111X(i, j) can be easily adjusted. In addition, a recombination region can be controlled easily.

### [Structure Example 2 of Mixed Material]

A material mixed with a phosphorescent substance can be used as the host material. When a fluorescent substance is used as the light-emitting substance, a phosphorescent substance can be used as an energy donor for supplying excitation energy to the fluorescent substance.

A mixed material containing a material to form an exciplex can be used as the host material. For example, a material forming an exciplex whose emission spectrum overlaps with the wavelength of the absorption band on the lowest energy side of the light-emitting substance can be used as the host material. This enables smooth energy transfer and improves emission efficiency. Alternatively, the driving voltage can be reduced.

A phosphorescent substance can be used as at least one of the materials forming an exciplex. Accordingly, reverse intersystem crossing can be used. Alternatively, triplet excitation energy can be efficiently converted into singlet excitation energy.

As the materials forming an exciplex, a material having a hole-transport property and a material having an electron-transport property are preferably combined, and the HOMO level of the material having a hole-transport property is preferably higher than or equal to the HOMO level of the material having an electron-transport property. Alternatively, the LUMO level of the material having a hole-transport property is preferably higher than or equal to the LUMO level of the material having an electron-transport property. In that case, an exciplex can be efficiently formed. Note that the LUMO levels and the HOMO levels of the materials can be derived from the electrochemical characteristics (the reduction potentials and the oxidation potentials). Specifically, the reduction potentials and the oxidation potentials can be measured by cyclic voltammetry (CV).

The formation of an exciplex can be confirmed by a phenomenon in which the emission spectrum of a mixed film in which the material having a hole-transport property and the material having an electron-transport property are mixed is shifted to a longer wavelength than the emission spectrum of each of the materials (or has another peak on the longer wavelength side) observed in comparison of the emission spectrum of the material having a hole-transport property, the emission spectrum of the material having an electron-transport property, and the emission spectrum of the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed in comparison of transient PL of the material having a hole-transport property, the transient PL of the material having an electron-transport property, and the transient PL of the mixed film of these materials. The transient PL can be rephrased as transient electroluminescence (EL). That is, the formation of an exciplex can also be confirmed by a difference in transient response observed in comparison of the transient EL of the material having a hole-transport property, the transient EL of the material having an electron-transport property, and the transient EL of the mixed film of these materials.

### <<Structure Example of Layer 112>>

For example, a material having a hole-transport property can be used for the layer 112. The layer 112 can be referred to as a hole-transport layer. A material having a wider band gap than a light-emitting material contained in the layer 111X(i,j) is preferably used for the layer 112. In that case, energy transfer from excitons generated in the layer 111X(i,j) to the layer 112 can be inhibited. Note that the structure of the layer 112 described in Embodiment 2 can be used for the layer 112.

### <<Structure Example of Layer 113>>

A material having an electron-transport property, a material having an anthracene skeleton, or a mixed material can be used for the layer 113, for example. The layer 113 can be referred to as an electron-transport layer. A material having a wider band gap than the light-emitting material contained in the layer 111X(i, j) is preferably used for the layer 113. In that case, energy transfer from excitons generated in the layer 111 X(i, j) to the layer 113 can be inhibited. Note that the structure of the layer 113 described in Embodiment 2 can be used for the layer 113.

Note that this embodiment can be combined with any of the other embodiments in this specification as appropriate.

### Embodiment 6

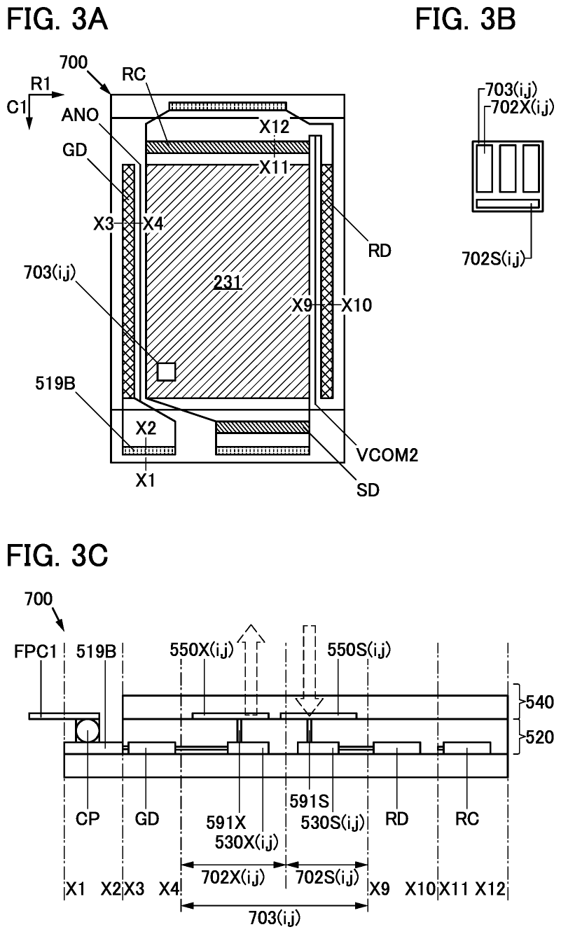

In this embodiment, a structure of a device of one embodiment of the present invention will be described with reference to FIG. 3 to FIG. 7.

FIG. 3 illustrates a structure of the device of one embodiment of the present invention. FIG. 3A is a top view of the device of one embodiment of the present invention, and FIG. 3B is a top view illustrating part of FIG. 3A. FIG. 3C is a cross-sectional view taken along a cutting line X1-X2, a cutting line X3-X4, a cutting line X9-X10, a cutting line X11-X12, and a pixel set 703(i,j) in FIG. 3A.

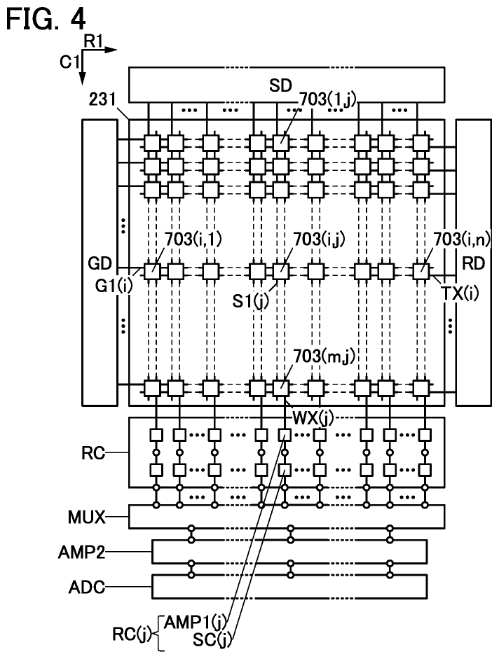

FIG. 4 is a block diagram illustrating the structure of the device of one embodiment of the present invention.

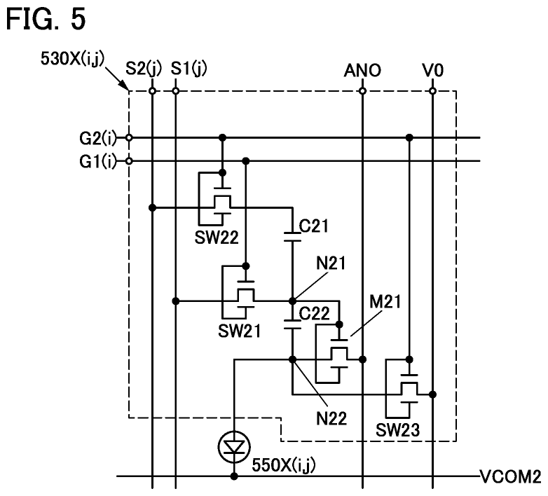

FIG. 5 is a circuit diagram illustrating the structure of the device of one embodiment of the present invention.

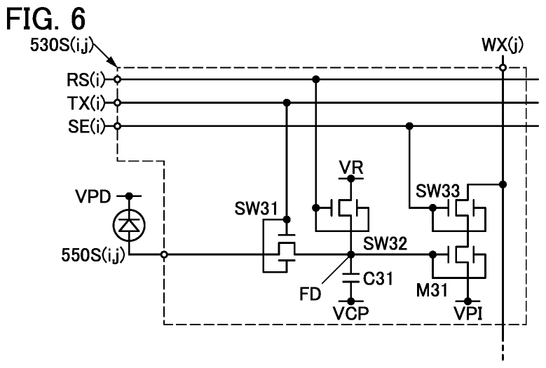

FIG. 6 is a circuit diagram illustrating the structure of the device of one embodiment of the present invention.

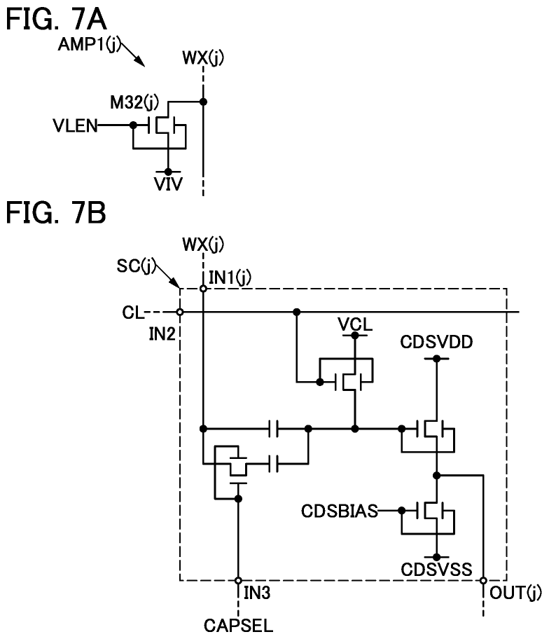

FIG. 7 shows circuit diagrams illustrating the structures of the device of one embodiment of the present invention. FIG. 7A is a circuit diagram illustrating an amplifier circuit AMP1 that can be used in the device of one embodiment of the present invention, and FIG. 7B is a circuit diagram of a sampling circuit SC(j) that can be used in the device of one embodiment of the present invention.

Note that in this specification, an integer variable of 1 or more is sometimes used in reference numerals. For example, (p) where p is an integer variable of 1 or more is sometimes used in part of a reference numeral that specifies any of p components at a maximum. As another example, (m, n) where m and n are each an integer variable of 1 or more is sometimes used in part of a reference numeral that specifies any of m×n components at a maximum.

### <Structure Example 1 of Device 700>

The device 700 of one embodiment of the present invention includes a region 231, a conductive film ANO, and a conductive film VCOM2 (see FIG. 3A). The region 231 includes the pixel set 703(i,j).

<<Structure Example 1 of Pixel Set 703(i,j)>>

The pixel set 703(i,j) includes a pixel 702X(i,j) (see FIG. 3B and FIG. 3C).

The pixel 702X(i, j) includes a pixel circuit 530X(i, j) and the light-emitting device 550X(i,j). The pixel circuit 530X(i,j) is electrically connected to the conductive film ANO (see FIG. 5).

One the electrode of the light-emitting device 550X(i,j) is electrically connected to the pixel circuit 530X(i,j), and the other electrode of the light-emitting device 550X(i,j) is electrically connected to the conductive film VCOM2. For example, an electrical connection is made through a conductive film provided in an opening portion 591X included in a functional layer 520. An electrical connection is also made through a conductive film provided in an opening portion 591S included in the functional layer 520. The device 700 includes a terminal 519B, a flexible printed circuit FPC1, and a conductive material CP.

For example, the light-emitting device described in Embodiment 5 can be used as the light-emitting device 550X(i,j). The device 700 has a function of displaying an image. The device 700 is a display device.

<<Structure Example 2 of Pixel Set 703(i,j)>>

The pixel set 703(i,j) includes a pixel 702S(i,j) (see FIG. 3B and FIG. 3C).

The pixel 702S(i,j) includes a pixel circuit 530S(i,j) and the photoelectric conversion device 550S(i,j). The pixel circuit 530S(i,j) is electrically connected to a conductive film WX(j) and has a function of supplying an imaging signal (see FIG. 6).

One electrode of the photoelectric conversion device 550S(i,j) is electrically connected to the pixel circuit 530S(i,j) and the other electrode of the photoelectric conversion device 550S(i,j) is electrically connected to a conductive film VPD.

For example, the photoelectric conversion device described in any of Embodiment 2 to Embodiment 4 can be used as the photoelectric conversion device 550S(i,j). The device 700 has a function of supplying an imaging signal. The device 700 is also an imaging device.

### <Structure Example 2 of Device 700>

The device 700 of one embodiment of the present invention includes a functional layer 540 and the functional layer 520 (see FIG. 3C). The functional layer 540 overlaps with the functional layer 520.

The functional layer 540 includes the light-emitting device 550X(i,j) and the photoelectric conversion device 550S(i,j).

The functional layer 520 includes the pixel circuit 530X(i,j), the conductive film ANO, and the conductive film VCOM2 (see FIG. 3C and FIG. 5).

The functional layer 520 includes the pixel circuit 530S(i,j), the conductive film WX(j), and the conductive film VPD (see FIG. 3C and FIG. 6).

### <Structure Example 3 of Device 700>

The device 700 of one embodiment of the present invention includes a driver circuit GD, a conductive film G1(i), and a conductive film G2(i) (see FIG. 4 and FIG. 5).

### <<Structure Example of Driver Circuit GD>>

The driver circuit GD supplies a first selection signal and a second selection signal.

The conductive film G1(i) is supplied with the first selection signal, and the conductive film G2(i) is supplied with the second selection signal.

### <Structure Example 4 of Device 700>

The device 700 of one embodiment of the present invention includes a driver circuit SD, a conductive film S1(j), and a conductive film S2(j) (see FIG. 4 and FIG. 5). The device 700 also includes a conductive film V0.

### <<Structure Example of Driver Circuit SD>>

The driver circuit SD supplies a first control signal and a second control signal.

The conductive film S1(j) is supplied with the first control signal, and the conductive film S2(j) is supplied with the second control signal.

### <<Structure Example 1 of Pixel Circuit 530X(i,j)>>

The pixel circuit 530X(i,j) is electrically connected to the conductive film G1(i) and the conductive film S1(j). The conductive film G1(i) supplies the first selection signal, and the conductive film S1(j) supplies the first control signal.

The pixel circuit 530X(i,j) drives the light-emitting device 550X(i,j) in response to the first selection signal and the first control signal. The light-emitting device 550X(i,j) emits light.

### <<Structure Example 2 of Pixel Circuit 530X(i,j)>>

The pixel circuit 530X(i,j) includes a switch SW21, a switch SW22, a transistor M21, a capacitor C21, and a node N21.

The transistor M21 includes a gate electrode electrically connected to the node N21, a first electrode electrically connected to the light-emitting device 550X(i,j), and a second electrode electrically connected to the conductive film ANO.

The switch SW21 includes a first terminal electrically connected to the node N21, a second terminal electrically connected to the conductive film S1(j), and a gate electrode having a function of controlling the conduction state or the non-conduction state on the basis of the potential of the conductive film G1(i).

The switch SW22 includes a first terminal electrically connected to the conductive film S2(j) and a gate electrode having a function of controlling the conduction state or the non-conduction state on the basis of the potential of the conductive film G2(i).

The capacitor C21 includes a conductive film electrically connected to the node N21 and a conductive film electrically connected to a second electrode of the switch SW22.

Thus, the image signal can be stored in the node N21. The potential of the node N21 can be changed using the switch SW22. Alternatively, the intensity of light emitted from the light-emitting device 550X(i,j) can be controlled with the potential of the node N21. As a result, a novel device that is highly convenient, useful, or reliable can be provided.

### <<Structure Example 3 of Pixel Circuit 530X(i,j)>>

The pixel circuit 530X(i,j) includes a switch SW23, a node N22, and a capacitor C22.

The switch SW23 includes a first terminal electrically connected to the conductive film V0, a second terminal electrically connected to the node N22, and a gate electrode having a function of controlling the conduction state or the non-conduction state on the basis of the potential of the conductive film G2(i).

The capacitor C22 includes a conductive film electrically connected to the node N21 and a conductive film electrically connected to the node N22.

Note that the first electrode of the transistor M21 is electrically connected to the node N22.

### <Structure Example 5 of Device 700>

The device 700 of one embodiment of the present invention includes a driver circuit RD, a conductive film RS(i), a conductive film TX(i), and a conductive film SE(i) (see FIG. 4 and FIG. 6).

### <<Structure Example of Driver Circuit RD>>

The driver circuit RD supplies a third selection signal, a fourth selection signal, and a fifth selection signal.

The conductive film RS(i) is supplied with the third selection signal, the conductive film TX(i) is supplied with the fourth selection signal, and the conductive film SE(i) is supplied with the fifth selection signal.

### <<Structure Example 1 of Pixel Circuit 530S(i,j)>>

The pixel circuit 530S(i,j) is electrically connected to the conductive film RS(i), the conductive film TX(i), and the conductive film SE(i). The conductive film RS(i) is supplied with the third selection signal, the conductive film TX(i) is supplied with the fourth selection signal, and the conductive film SE(i) is supplied with the fifth selection signal.

The pixel circuit 530S(i,j) is initialized in response to the third selection signal, performs imaging in response to the fourth selection signal, and supplies an imaging signal in response to the fifth selection signal. Imaging can be performed in a period during which the light-emitting device 550X(i,j) emits light.

### <<Structure Example 2 of Pixel Circuit 530S(i,j)>>

The pixel circuit 530S(i,j) includes a switch SW31, a switch SW32, a switch SW33, a transistor M31, a capacitor C31, and a node FD.

The switch SW31 includes a first terminal electrically connected to the photoelectric conversion device 550S(i,j), a second terminal electrically connected to the node FD, and a gate electrode having a function of controlling the conduction state or the non-conduction state on the basis of a potential of the conductive film TX(i).

The switch SW32 includes a first terminal electrically connected to the node FD, a second terminal electrically connected to a conductive film VR, and a gate electrode having a function of controlling the conduction state or the non-conduction state on the basis of a potential of the conductive film RS(i).

The capacitor C31 includes a conductive film electrically connected to the node FD and a conductive film electrically connected to a conductive film VCP.

The transistor M31 includes a gate electrode electrically connected to the node FD and a first electrode electrically connected to a conductive film VPI.

The switch SW33 includes a first terminal electrically connected to a second electrode of the transistor M31, a second terminal electrically connected to the conductive film WX(j), and a gate electrode having a function of controlling the conduction state or the non-conduction state on the basis of a potential of the conductive film SE(i).

Thus, an imaging signal generated by the photoelectric conversion device 550S(i,j) can be transferred to the node FD using the switch SW31. The imaging signal generated by the photoelectric conversion device 550S(i,j) can be stored in the node FD using the switch SW31. Electrical continuity between the pixel circuit 530S(i,j) and the photoelectric conversion device 550S(i,j) can be broken by the switch SW31. A correlated double sampling method can be used. Noise included in the imaging signal can be reduced. As a result, a novel device that is highly convenient, useful, or reliable can be provided.

### <Structure Example 6 of Device 700>

The device 700 of one embodiment of the present invention includes a reading circuit RC, a conductive film CL, and a conductive film CAPSEL (see FIG. 4, FIG. 7A, and FIG. 7B).

The device 700 also includes a conductive film VLEN and a conductive film VIV.

The device 700 also includes a conductive film VCL, a conductive film CDSVDD, a conductive film CDSVSS, and a conductive film CDSBIAS.

### <<Structure Example of Reading Circuit RC>>

The reading circuit RC includes a reading circuit RC(j) (see FIG. 4).

The reading circuit RC(j) includes an amplifier circuit AMP1(j) and a sampling circuit SC(j).

### [Structure Example 1 of Amplifier Circuit AMP1(j)]

The amplifier circuit AMP1(j) is electrically connected to the conductive film WX(j) and has a function of amplifying an imaging signal.

### [Structure Example 2 of Amplifier Circuit AMP1(j)]

The amplifier circuit AMP1(j) includes a transistor M32(j), and the transistor M32(j) includes a gate electrode electrically connected to the conductive film VLEN, a first electrode electrically connected to the conductive film WX(j), and a second electrode electrically connected to the conductive film VIV.

Note that the conductive film WX(j) connects the transistor M31 and the transistor M32(j) when the switch SW33 is in a conduction state (see FIG. 6 and FIG. 7A). Thus, a source follower circuit can be configured with the transistor M31 and the transistor M32(j). The potential of the conductive film WX(j) can be changed on the basis of the potential of the node FD.

### <<Structure Example of Sampling Circuit SC(j)>>

The sampling circuit SC(j) includes a terminal IN1(j), a terminal IN2, a terminal IN3, and a terminal OUT(j) (see FIG. 7B).

The terminal IN1(j) is electrically connected to the conductive film WX(j), the terminal IN2 is electrically connected to the conductive film CL, and the terminal IN3 is electrically connected to the conductive film CAPSEL.

The sampling circuit SC(j) has a function of obtaining an imaging signal according to the potentials of the conductive film CL and the conductive film CAPSEL. The terminal OUT(j) has a function of supplying a signal which changes according to the potential of the terminal IN1(j).

Thus, an imaging signal can be obtained from the pixel circuit 530S(i,j). A correlated double sampling method can be employed, for example. The sampling circuit SC(j) can be provided for each conductive film WX(j). A differential signal of the pixel circuit 530S(i,j) can be obtained by the corresponding conductive film WX(j). The operating frequency of the sampling circuit SC(j) can be low. Noise can be reduced. As a result, a novel device that is highly convenient, useful, or reliable can be provided.

### <Structure Example 7 of Device 700>

The device 700 of one embodiment of the present invention includes the region 231 (see FIG. 4). The region 231 has a function of displaying an image.

The region 231 includes a group of pixels 703(i, 1) to 703(i, n) and a different group of pixels 703(1, j) to 703(m, j).

The group of pixels 703(i,1) to 703(i,n) is provided in the row direction (the direction indicated by an arrow R1 in the drawing), and the group of pixels 703(i,1) to 703(i,n) includes the pixel 703(i,j).

The conductive film G1(i) is electrically connected to the group of pixels 703(i, 1) to 703(i, n).

The different group of pixels 703(1j) to 703(m,j) is provided in the column direction (the direction indicated by an arrow C1 in the drawing) intersecting the row direction, and the different group of pixels 703(1,j) to 703(m,j) includes the pixel 703(i,j).

The different group of pixels 703(1j) to 703(m,j) is electrically connected to the conductive film S1g(j).

### <Structure Example 8 of Device 700>

The device 700 of one embodiment of the present invention includes a multiplexer MUX, an amplifier circuit AMP2, and an analog-digital converter circuit ADC (see FIG. 4).

The multiplexer MUX has a function of obtaining an imaging signal from one selected from the plurality of sampling circuits SC(j) and supplying the imaging signal to the amplifier circuit AMP2, for example.

Thus, imaging data can be obtained by selecting a predetermined pixel from a plurality of pixels arranged in the row direction. The number of imaging signals obtained at the same time can be limited to a predetermined number. It is possible to use the analog-digital converter circuit ADC in which the number of input channels is smaller than the number of pixels arranged in the row direction. As a result, a novel display device that is highly convenient, useful, or reliable can be provided.

The amplifier circuit AMP2 can amplify the imaging signal and supply the amplified signal to the analog-digital converter circuit ADC.

Note that this embodiment can be combined with any of the other embodiments in this specification as appropriate.

### Embodiment 7

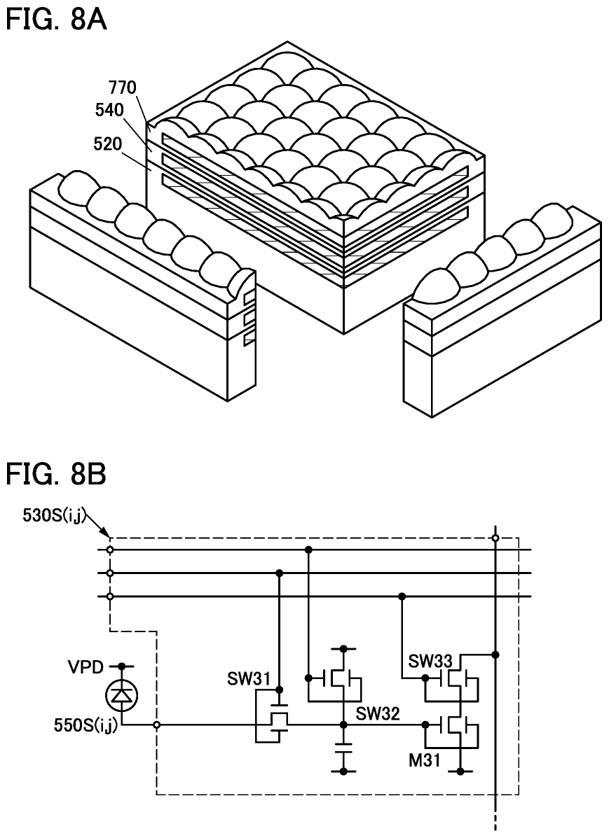

In this embodiment, a structure of a device of one embodiment of the present invention will be described with reference to FIG. 8 to FIG. 10.

FIG. 8A is a cross-sectional perspective view illustrating an imaging device, and FIG. 8B is a circuit diagram illustrating a pixel circuit.

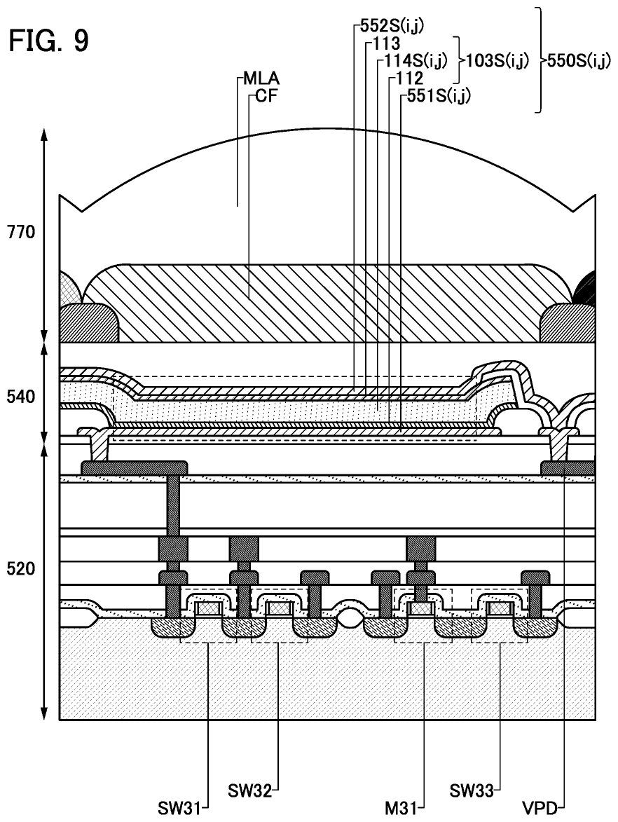

FIG. 9 is a cross-sectional view illustrating the structure of the device of one embodiment of the present invention. Specifically, FIG. 9 is a cross-sectional view of a pixel.

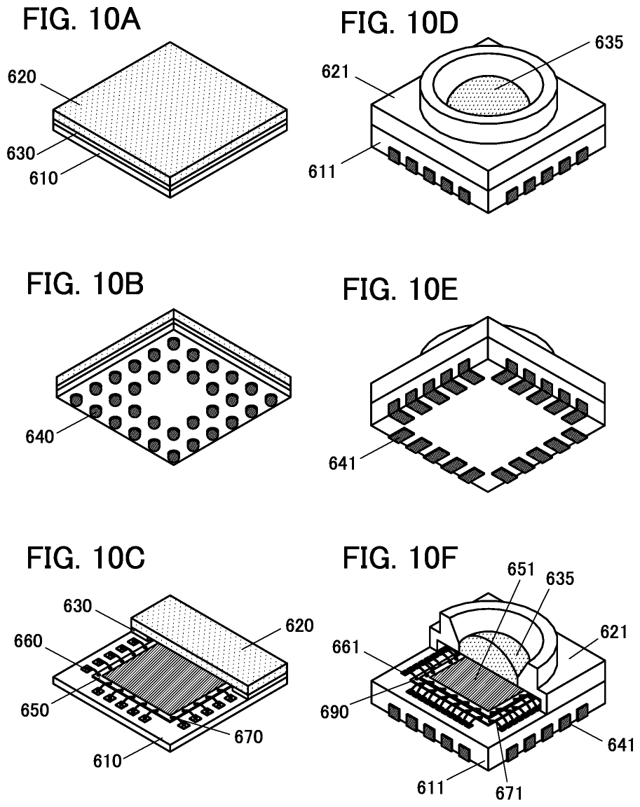

FIG. 10A to FIG. 10F are perspective views of a package and a module each including the imaging device.

### <Structure Example of Imaging Device>

The device of one embodiment of the present invention includes the functional layer 520, the functional layer 540, and a functional layer 770 (see FIG. 8). The functional layer 540 is held between the functional layer 520 and the functional layer 770 (see FIG. 8 and FIG. 9).

The functional layer 540 includes the photoelectric conversion device 550S(i,j) (see FIG. 9).

The functional layer 520 includes the pixel circuit 530S(i,j). The device of one embodiment of the present invention has an imaging function. The device of one embodiment of the present invention can be referred to as an imaging device.

The functional layer 770 includes, for example, a microlens array MLA and a coloring layer CF.

The imaging device of one embodiment of the present invention includes a pixel and the conductive film VPD (see FIG. 9). The pixel includes the photoelectric conversion device 550S(i,j) and a pixel circuit 530S(i,j). The photoelectric conversion device 550S(i,j) includes the electrode 551S(i,j) and an electrode 552S(i,j). The electrode 551S(i,j) is electrically connected to the pixel circuit 530S(i,j), and the electrode 552S(i,j) is electrically connected to the conductive film VPD (see FIG. 8B and FIG. 9).

### <<Photoelectric Conversion Device 550S(i,j)>>

The photoelectric conversion device 550S(i,j) includes the unit 103S(i,j) (see FIG. 9). The unit 103S(i,j) is held between the electrode 552S(i,j) and the electrode 551S(i,j).

The unit 103S(i,j) includes the layer 114S(i,j), the layer 112, and the layer 113. The layer 114S(i,j) is held between the layer 113 and the layer 112, the layer 113 is held between the electrode 552S(i,j) and the layer 114S(i,j), and the layer 112 is held between the layer 114S(i,j) and the electrode 551S(i,j).

For example, the photoelectric conversion device described in any of Embodiment 2 to Embodiment 4 can be used as the photoelectric conversion device 550S(i,j).

### <<Pixel Circuit 530S(i,j)>>

The pixel circuit 530S(i,j) includes the switch SW31, the switch SW32, the switch SW33, and the transistor M31 (see FIG. 8B and FIG. 9). As the transistor M31, a transistor formed using a silicon substrate can be used, for example.

### <Structure Example 2 of Imaging Device>

Examples of a package and a camera module in each of which an image sensor chip is placed will be described.

FIG. 10A is an external perspective view of the top surface side of a package in which an image sensor chip is placed. The package includes a package substrate 610 to which an image sensor chip 650 is fixed, a cover glass 620, an adhesive 630 for bonding them, and the like.

FIG. 10B is an external perspective view of the bottom surface side of the package. A BGA (Ball grid array) in which solder balls are used as bumps 640 on the bottom surface of the package is employed. Note that, other than the BGA, an LGA (Land grid array), a PGA (Pin Grid Array), or the like may be employed.

FIG. 10C is a perspective view of the package, in which parts of the cover glass 620 and the adhesive 630 are not illustrated. Electrode pads 660 are formed over the package substrate 610, and the electrode pads 660 and the bumps 640 are electrically connected to each other via through-holes. The electrode pads 660 are electrically connected to the image sensor chip 650 through wires 670.

FIG. 10D is an external perspective view of the top surface side of a camera module in which an image sensor chip is placed in a package with a built-in lens. The camera module includes a package substrate 611 to which an image sensor chip 651 is fixed, a lens cover 621, a lens 635, and the like. Furthermore, an IC chip 690 having functions of a driver circuit, a signal conversion circuit, and the like of the imaging device is provided between the package substrate 611 and the image sensor chip 651; thus, the structure as an SiP (System in package) is included.

FIG. 10E is an external perspective view of the bottom surface side of the camera module. A QFN (Quad flat no-lead package) structure where lands 641 for mounting are provided on the bottom and side surfaces of the package substrate 611 is employed. Note that this structure is only an example, and a QFP (Quad flat package) or the above-mentioned BGA may also be provided.

FIG. 10F is a perspective view of the module, in which parts of the lens cover 621 and the lens 635 are not illustrated. The lands 641 are electrically connected to electrode pads 661, and the electrode pads 661 are electrically connected to the image sensor chip 651 or the IC chip 690 through wires 671.

The image sensor chip placed in a package having the above form can be easily mounted on a printed circuit board and the like; thus, the image sensor chip can be incorporated into a variety of semiconductor apparatuses and electronic devices.

This embodiment can be combined with any of the other embodiments and examples as appropriate.

### Embodiment 8

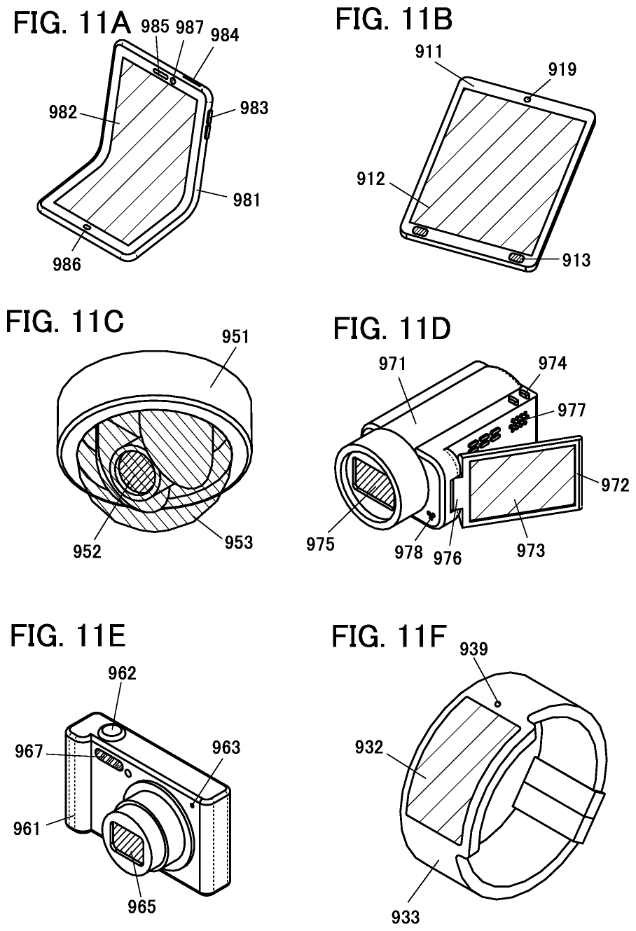

As electronic devices that can use the imaging device of one embodiment of the present invention, display devices, personal computers, image memory devices or image reproducing devices provided with storage media, mobile phones, game machines including portable game machines, portable data terminals, e-book readers, cameras such as video cameras and digital still cameras, goggle-type displays (head mounted displays), navigation systems, audio reproducing devices (car audio players, digital audio players, and the like), copiers, facsimiles, printers, multifunction printers, automated teller machines (ATM), vending machines, and the like are given. FIG. 11A to FIG. 11F illustrate specific examples of such electronic devices.

FIG. 11A is an example of a mobile phone, which includes a logic board and a battery in addition to a housing 981, a display portion 982, an operation button 983, an external connection port 984, a speaker 985, a microphone 986, a camera 987, and the like. The display portion 982 includes a display module. The display module includes a display device and either a connector or an integrated circuit. The display module is electrically connected to the logic board. The display portion 982 of the mobile phone is provided with a touch sensor. All operations including making a call and inputting text can be performed by touch on the display portion 982 with a finger, a stylus, or the like. The imaging device of one embodiment of the present invention and the operation method thereof can be used for obtaining an image in the mobile phone.

FIG. 11B is a portable data terminal, which includes a logic board and a battery in addition to a housing 911, a display portion 912, a speaker 913, a camera 919, and the like. The display portion 912 includes a display module, and the display module includes a display device and either a connector or an integrated circuit. The display module is electrically connected to the logic board. A touch panel function of the display portion 912 enables input and output of information. Furthermore, a character or the like in an image that is captured by the camera 919 can be recognized and the character can be voice-output from the speaker 913. The imaging device of one embodiment of the present invention and the operation method thereof can be used for obtaining an image in the portable data terminal.

FIG. 11C is a surveillance camera, which includes a logic board in addition to a support base 951, a camera unit 952, a protection cover 953, and the like. By setting the camera unit 952 provided with a rotating mechanism and the like on a ceiling, an image of all the surroundings can be taken. The imaging device of one embodiment of the present invention and the operation method thereof can be used for obtaining an image in the camera unit. Note that a surveillance camera is a name in common use and does not limit the use thereof. A device that has a function of a surveillance camera is also called a camera or a video camera, for example.

FIG. 11D is a video camera, which includes a logic board and a battery in addition to a first housing 971, a second housing 972, a display portion 973, an operation key 974, a lens 975, a connection portion 976, a speaker 977, a microphone 978, and the like. The operation key 974 and the lens 975 are provided for the first housing 971, and the display portion 973 is provided for the second housing 972. The display portion 973 includes a display module. The display module includes a display device and a connector, or an integrated circuit, and the display module is electrically connected to the logic board. The imaging device of one embodiment of the present invention and the operation method thereof can be used for obtaining an image in the video camera.

FIG. 11E is a digital camera, which includes a logic board and a battery in addition to a housing 961, a shutter button 962, a microphone 963, a light-emitting portion 967, a lens 965, and the like. The imaging device of one embodiment of the present invention and the operation method thereof can be used for obtaining an image in the digital camera.

FIG. 11F is a wrist-watch-type information terminal, which includes a logic board and a battery in addition to a display portion 932, a housing and wristband 933, a camera 939, and the like. The display portion 932 is provided with a touch panel for performing the operation of the information terminal. The display portion 932 includes a display module. The display module includes a display device and a connector, or an integrated circuit, and the display module is electrically connected to the logic board. The display portion 932 and the housing and wristband 933 have flexibility and fit a body well. The imaging device of one embodiment of the present invention and the operation method thereof can be used for obtaining an image in the information terminal.

This embodiment can be combined with any of the other embodiments and examples as appropriate.

### Example 1

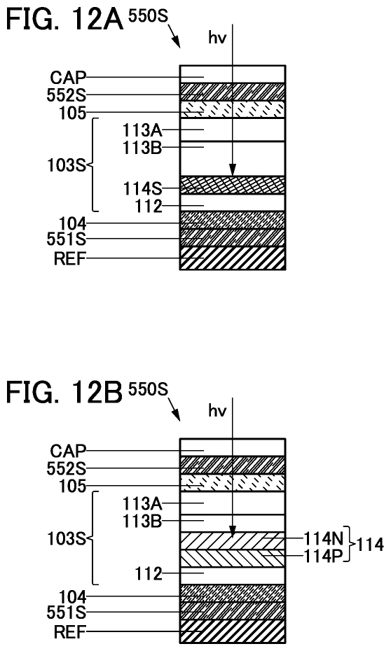

In this example, a device 1, a device 2, a device 3, and a device 4 of one embodiment of the present invention will be described with reference to FIG. 12 to FIG. 15.

FIG. 12A is a diagram illustrating a structure of the photoelectric conversion device 550S.

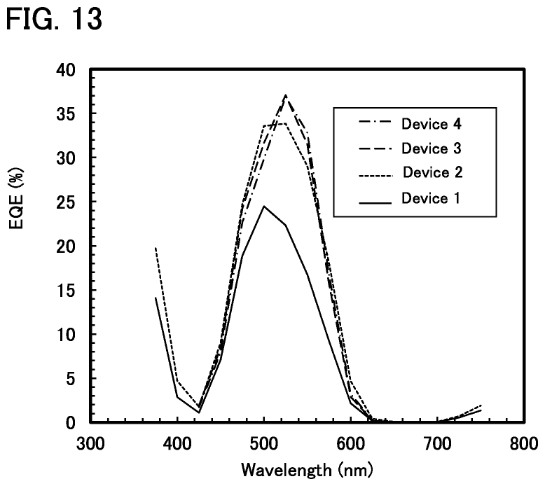

FIG. 13 is a graph showing spectral sensitivity characteristics of the device 1, the device 2, the device 3, and the device 4.

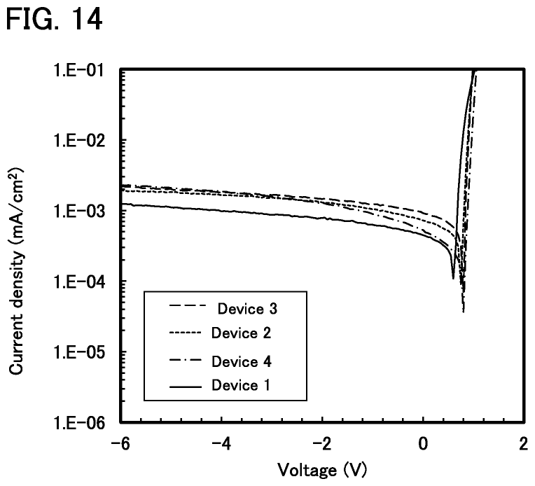

FIG. 14 is a graph showing voltage-current density characteristics of the device 1, the device 2, the device 3, and the device 4 in a state where light irradiation was performed.

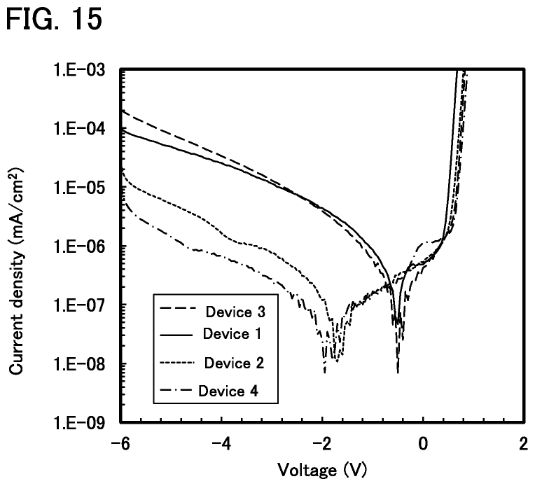

FIG. 15 is a graph showing voltage-current density characteristics of the device 1, the device 2, the device 3, and the device 4 in a state where light irradiation was not performed.

### <Device 1>

The fabricated device 1, which is described in this example, has a structure similar to that of the photoelectric conversion device 550S (see FIG. 12).

The photoelectric conversion device 550S includes the electrode 551S, the electrode 552S, the layer 112, the layer 114S, and the layer 113. The layer 112 is held between the electrode 551S and the electrode 552S, the layer 114S is held between the electrode 552S and the layer 112, the layer 113 is held between the electrode 552S and the layer 114S, and the layer 113 has higher electron mobility than the layer 112.

### <<Structure of Device 1>>

Table 1 shows the structure of the device 1. Structural formulae of materials used for the devices described in this example are shown below. Note that in the tables in this example, subscript characters and superscript characters are written in ordinary size for convenience. For example, subscript characters in abbreviations and superscript characters in units are written in ordinary size in the tables. Such notations in the tables can be replaced by referring to the description in the specification.

TABLE 1

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114S | DPAPhA:EtHex-PTCDI | 0.2:0.8 | 60 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

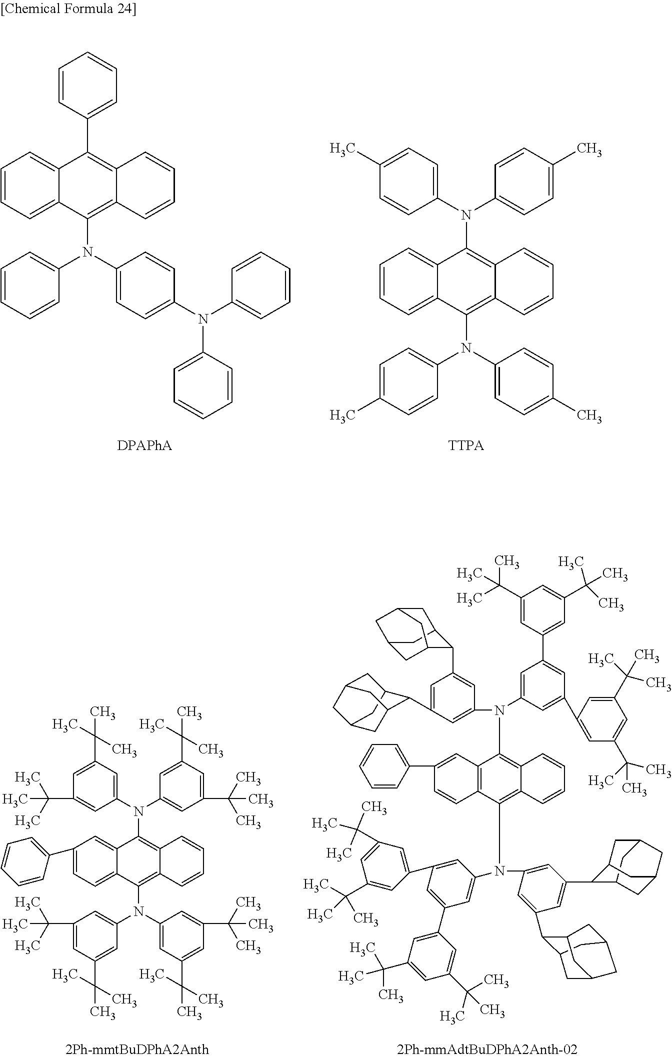

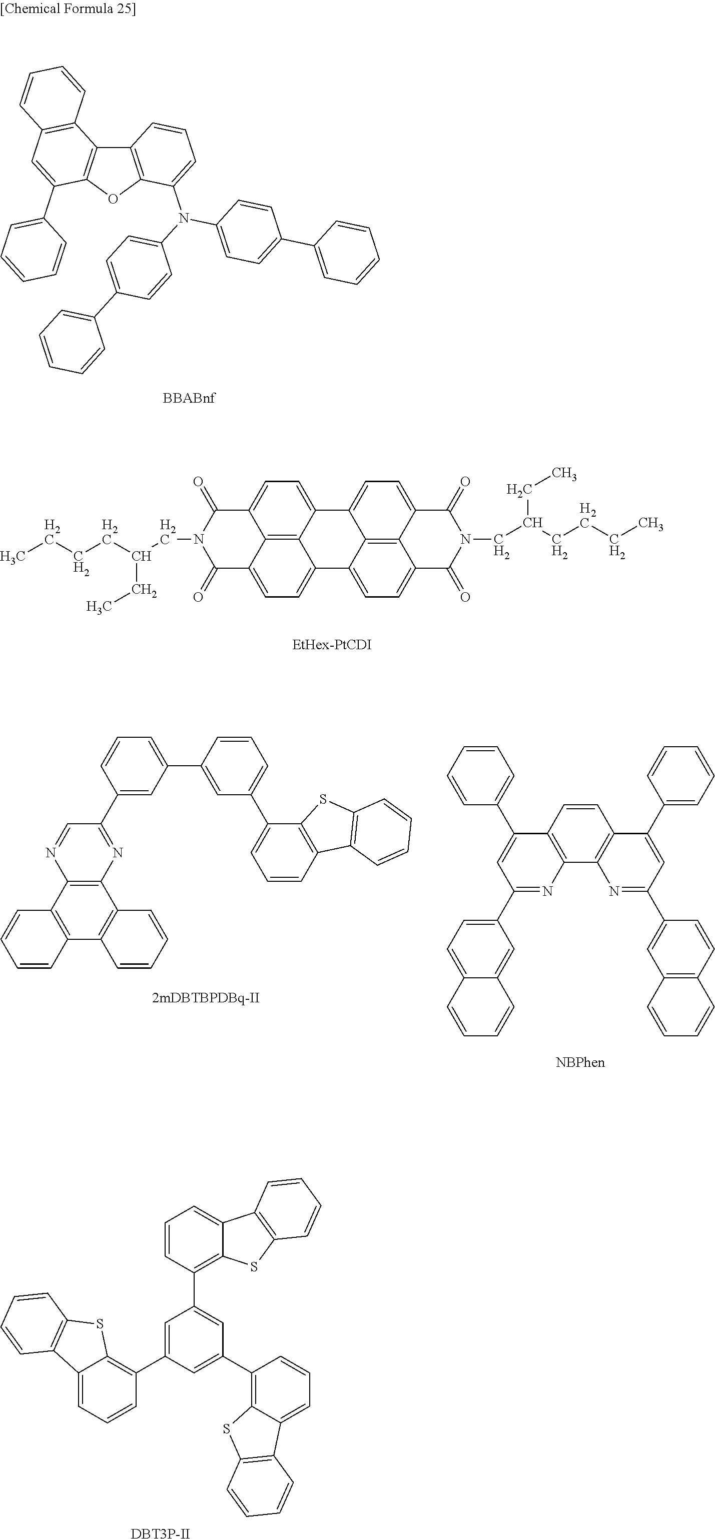 [SMILES: c1ccc(-c2ccc(N(c3ccc(-c4ccccc4)cc3)c3cccc4c3oc3c(-c5ccccc5)cc5ccccc5c34)cc2)cc1 ; CCCCC(CC)CN1C(=O)c2ccc3c4ccc5c6c(ccc(c7ccc(c2c37)C1=O)c64)C(=O)N(CC(CC)CCCC)C5=O ; c1cc(-c2cccc(-c3cccc4c3sc3ccccc34)c2)cc(-c2cnc3c4ccccc4c4ccccc4c3n2)c1 ; c1ccc(-c2cc(-c3ccc4ccccc4c3)nc3c2ccc2c(-c4ccccc4)cc(-c4ccc5ccccc5c4)nc23)cc1 ; c1ccc2c(c1)sc1c(-c3cc(-c4cccc5c4sc4ccccc45)cc(-c4cccc5c4sc4ccccc45)c3)cccc12 ; "DBT3P-II"]

### <<Fabrication Method of Device 1>>

The device 1 described in this example was fabricated using a method including the following steps.

### [First Step]

In a first step, the reflective film REF was formed. Specifically, the reflective film REF was formed by a sputtering method using an alloy containing silver (Ag), palladium (Pd), and copper (Cu) (abbreviation: APC) as a target.

The reflective film REF contains APC and has a thickness of 100 nm.

### [Second Step]

In a second step, the electrode 551S was formed over the reflective film REF. Specifically, the electrode 551S was formed by a sputtering method using indium oxide-tin oxide containing silicon or silicon oxide (abbreviation: ITSO) as a target.

Note that the electrode 551S contains ITSO and has a thickness of 100 nm and an area of 4 mm² (2 mm×2 mm).

Next, a base material over which the electrode 551S was formed was washed with water, baked at 200° C. for an hour, and then subjected to UV ozone treatment for 370 seconds. After that, the base material was transferred into a vacuum evaporation apparatus where the inside pressure was reduced to approximately 10⁻⁴ Pa, and vacuum baking was performed at 170° C. for 30 minutes in a heating chamber of the vacuum evaporation apparatus. Then, the base material was cooled down for approximately 30 minutes.

### [Third Step]

In a third step, the layer 104 was formed over the electrode 551S. Specifically, materials were co-evaporated by a resistance-heating method.

Note that the layer 104 contains N,N-bis(4-biphenyl)-6-phenylbenzo[b]naphtho[1,2-d]furan-8-amine (abbreviation: BBABnf) and an electron acceptor material (abbreviation: OCHD-003) at BBABnf:OCHD-003=1:0.1 (weight ratio) and has a thickness of 11 nm. Note that OCHD-003 contains fluorine, and has a molecular weight of 672.

### [Fourth Step]

In a fourth step, the layer 112 was formed over the layer 104. Specifically, a material was evaporated by a resistance-heating method.

The layer 112 contains BBABnf and has a thickness of 40 nm.

### [Fifth Step]

In a fifth step, the layer 114S was formed over the layer 112. Specifically, materials were co-evaporated by a resistance-heating method.

Note that the layer 114S contains N-phenyl-N-[4-(diphenylamino)-phenyl]-10-phenyl-9-anthracenamine (abbreviation: DPAPhA) and N,N′-bis(2-ethylhexyl)-3,4,9,10-perylenetetracarboxylic diimide (abbreviation: EtHex-PTCDI) at DPAPhA:EtHex-PTCDI=0.2:0.8 (weight ratio) and has a thickness of 60 nm.

### [Sixth Step]

In a sixth step, the layer 113B was formed over the layer 114S. Specifically, a material was evaporated by a resistance-heating method.

Note that the layer 113B contains 2-[3-(3′-dibenzothiophen-4-yl)biphenyl]dibenzo[f,h]quinoxaline (abbreviation: 2mDBTBPDBq-II) and has a thickness of 10 nm.

### [Seventh Step]

In a seventh step, the layer 113B was formed over the layer 113A. Specifically, a material was evaporated by a resistance-heating method.

The layer 113A contains 2,9-di(2-naphthyl)-4,7-diphenyl-1,10-phenanthroline (abbreviation: NBPhen) and has a thickness of 10 nm.

### [Eighth Step]

In an eighth step, the layer 105 was formed over the layer 113A. Specifically, a material was evaporated by a resistance-heating method.

Note that the layer 105 contains lithium fluoride (abbreviation: LiF) and has a thickness of 1 nm.

### [Ninth Step]

In a ninth step, the electrode 552S was formed over the layer 105. Specifically, materials were co-evaporated by a resistance-heating method.

Note that the electrode 552S contains silver (abbreviation: Ag) and magnesium (abbreviation: Mg) at Ag:Mg=3:0.3 (weight ratio) and has a thickness of 10 nm.

### [Tenth Step]

In a tenth step, a layer CAP was formed over the electrode 552S. Specifically, a material was evaporated by a resistance-heating method.

The layer CAP contains 4,4′,4″-(benzene-1,3,5-triyl)tri(dibenzothiophene) (abbreviation: DBT3P-II) and has a thickness of 80 nm.

### <<Operation Characteristics of Device 1>>

The operation characteristics of the device 1 were measured at room temperature (See FIG. 13 to FIG. 15).

In the state where a potential of −4 V relative to the potential of the electrode 552S was supplied to the electrode 551S, irradiation of monochromatic light was performed. The current density with respect to the amount of irradiation light was measured, and the external quantum efficiency (EQE) was calculated from the conversion efficiency (see FIG. 13). Note that the selected wavelengths of the monochromatic light were in steps of 25 nm in the range of 375 nm to 750 nm inclusive.

The density of current flowing through the device was measured while the potential of the electrode 551S was swept from −6 V to +2 V using the potential of the electrode 552S as a reference, in the state where the device was irradiated with 550-nm monochromatic light with an intensity of 12.5 μW/cm² (see FIG. 14).

In addition, the density of dark current flowing through the device was measured while the potential of the electrode 552S was swept from −6 V to +2 V using the potential of the electrode 552S as a reference, in the state where the device was irradiated with no light (see FIG. 15). Table 102 shows the characteristics of the device 1 and the other devices to be described later. Note that light enabling the highest EQE in the measurement range was used for the irradiation light.

TABLE 2

| | | Wavelength |
|---|---|---|
| | | of irradiation |
| | EQE/% | light/nm |
| Device 1 | 24.5 | @500 nm |
| Device 2 | 33.8 | @525 nm |
| Device 3 | 37.1 | @525 nm |
| Device 4 | 36.9 | @525 nm |
| Device 5 | 6.94 | @500 nm |
| Device 6 | 11.31 | @475 nm |
| Device 7 | 7.525 | @500 nm |
| Device 8 | 10.42 | @500 nm |

The device 1 was found to have favorable photoelectric conversion characteristics. For example, the device 1 had an EQE of greater than or equal to 1% and less than 25% with respect to light with a wavelength of greater than or equal to 425 nm and less than or equal to 600 nm (see FIG. 13). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 14 and FIG. 15).

### <Device 2>

The fabricated device 2, which is described in this example, has a structure similar to that of the photoelectric conversion device 550S (see FIG. 12A). The structure of the device 2 is different from that of the device 1 in the layer 114S. Specifically, the device 2 is different from the device 1 in that the layer 114S contains 9,10-bis[N,N-di-(p-tolyl)-amino]anthracene (abbreviation: TTPA) instead of DPAPhA.

### <<Structure of Device 2>>

Table 3 shows the structure of the device 2.

TABLE 3

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114S | TTPA:EtHex-PTCDI | 0.2:0.8 | 60 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 2>>

The device 2 described in this example was fabricated using a method including the following steps.

Note that the fabrication method of the device 2 is different from the fabrication method of the device 1 in that TPPA and EtHex-PTCDI were co-evaporated instead of DPAPhA and EtHex-PTCDI in the fifth step. Different portions are described in detail here, and the above description is referred to for portions formed by a similar method.

### [Fifth Step]

In the fifth step, the layer 114S was formed over the layer 112. Specifically, the materials were co-evaporated by a resistance-heating method.

Note that the layer 114S contains TPPA and EtHex-PTCDI at TPPA:EtHex-PTCDI=0.2:0.8 (weight ratio) and has a thickness of 60 nm.

### <<Operation Characteristics of Device 2>>

The operation characteristics of the device 2 were measured at room temperature (see FIG. 13 to FIG. 15).

Table 102 shows the main initial characteristics of the fabricated device.

The device 2 was found to have favorable photoelectric conversion characteristics. For example, the device 2 had an EQE of greater than or equal to 1% and less than 34% with respect to light with a wavelength of greater than or equal to 425 nm and less than or equal to 600 nm (see FIG. 13). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 14 and FIG. 15).

### <Device 3>

The fabricated device 3, which is described in this example, has a structure similar to that of the photoelectric conversion device 550S (see FIG. 12). The structure of the device 3 is different from that of the device 1 in the layer 114S. Specifically, the device 3 is different from the device 1 in that the layer 114S contains N,N′-(2-phenylanthracene-9,10-diyl)-N,N,N′,N′-tetrakis(3,5-di-tert-butylphenyl)diamine (abbreviation: 2Ph-mmtBuDPhA2Anth) instead of DPAPhA.

### <<Structure of Device 3>>

Table 4 shows the structure of the device 3.

TABLE 4

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114S | 2Ph-mmtBuDPhA2Anth:EtHex-PTCDI | 0.2:0.8 | 60 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 3>>

The device 3 described in this example was fabricated using a method including the following steps.

Note that the fabrication method of the device 3 is different from the fabrication method of the device 1 in that 2Ph-mmtBuDPhA2Anth and EtHex-PTCDI were co-evaporated instead of DPAPhA and EtHex-PTCDI in the fifth step. Different portions are described in detail here, and the above description is referred to for portions formed by a similar method.

### [Fifth Step]

In the fifth step, the layer 114S was formed over the layer 112. Specifically, the materials were co-evaporated by a resistance-heating method.

Note that the layer 114S contains 2Ph-mmtBuDPhA2Anth and EtHex-PTCDI at 2Ph-mmtBuDPhA2Anth:EtHex-PTCDI=0.2:0.8 (weight ratio) and has a thickness of 60 nm.

### <<Operation Characteristics of Device 3>>

The operation characteristics of the device 3 were measured at room temperature (see FIG. 13 to FIG. 15).

Table 2 shows main initial characteristics of the fabricated device.

In the state where a potential of −4V relative to the potential of the electrode 552S was supplied with to the electrode 551S, irradiation of monochromatic light was performed. The current density with respect to the amount of irradiation light was measured, and the EQE was calculated from the conversion efficiency (see FIG. 13). Note that the selected wavelengths of the monochromatic light were in steps of 25 nm in the range of 425 nm to 625 nm inclusive.

The device 3 was found to have favorable photoelectric conversion characteristics. For example, the device 3 had an EQE of greater than or equal to 1% and less than 38% with respect to light with a wavelength of greater than or equal to 425 nm and less than or equal to 600 nm (see FIG. 13). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 14 and FIG. 15).

### <Device 4>

The fabricated device 4, which is described in this example, has a structure similar to that of the photoelectric conversion device 550S (see FIG. 12). The structure of the device 4 is different from that of the device 1 in the layer 114S. Specifically, the device 4 is different from the device 1 in that the layer 114S contains N,N′-bis[3,5-bis(2-adamantyl)phenyl]-N,N′-bis[3,5-bis(3,5-di-tert-butylphenyl)phenyl]-2-phenylanthracene-9,10-diamine (abbreviation: 2Ph-mmAdtBuDPhA2Anth-02) instead of DPAPhA.

### <<Structure of Device 4>>

Table 5 shows the structure of the device 4.

TABLE 5

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114S | 2Ph-mmAdtBuDPhA2Anth-02:EtHex-PTCDI | 0.2:0.8 | 60 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 4>>

The device 4 described in this example was fabricated using a method including the following steps.

Note that the fabrication method of the device 4 is different from the fabrication method of the device 1 in that 2Ph-mmAdtBuDPhA2Anth-02 and EtHex-PTCDI were co-evaporated instead of DPAPhA and EtHex-PTCDI in the fifth step. Different portions are described in detail here, and the above description is referred to for portions formed by a similar method.

### [Fifth Step]

In the fifth step, the layer 114S was formed over the layer 112. Specifically, the materials were co-evaporated by a resistance-heating method.

Note that the layer 114S contains 2Ph-mmAdtBuDPhA2Anth-02 and EtHex-PTCDI at 2Ph-mmAdtBuDPhA2Anth-02:EtHex-PTCDI=0.2:0.8 (weight ratio) and has a thickness of 60 nm.

### <<Operation Characteristics of Device 4>>

The operation characteristics of the device 4 were measured at room temperature (see FIG. 13 to FIG. 15).

In the state where a potential of −4V relative to the potential of the electrode 552S was supplied to the electrode 551S, irradiation of monochromatic light was performed. The current density with respect to the amount of irradiation light was measured, and the EQE was calculated from the conversion efficiency (see FIG. 13). Note that the selected wavelengths of the monochromatic light were in steps of 25 nm in the range of 425 nm to 625 nm inclusive.

Table 2 shows main initial characteristics of the fabricated device.

The device 4 was found to have favorable photoelectric conversion characteristics. For example, the device 4 had an EQE of greater than or equal to 1% and less than 37% with respect to light with a wavelength of greater than or equal to 425 nm and less than or equal to 600 nm (see FIG. 13). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 14 and FIG. 15).

### Example 2

In this example, a device 5, a device 6, a device 7, and a device 8 of one embodiment of the present invention will be described with reference to FIG. 12 and FIG. 16 to FIG. 18.

FIG. 12B is a diagram illustrating a structure of the photoelectric conversion device 550S.

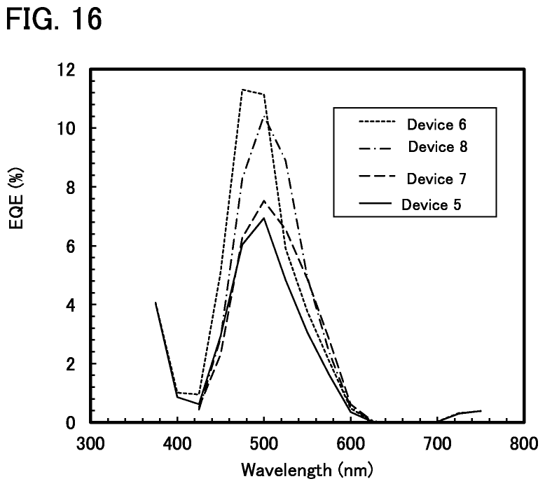

FIG. 16 is a graph showing spectral sensitivity characteristics of the device 5, the device 6, the device 7, and the device 8.

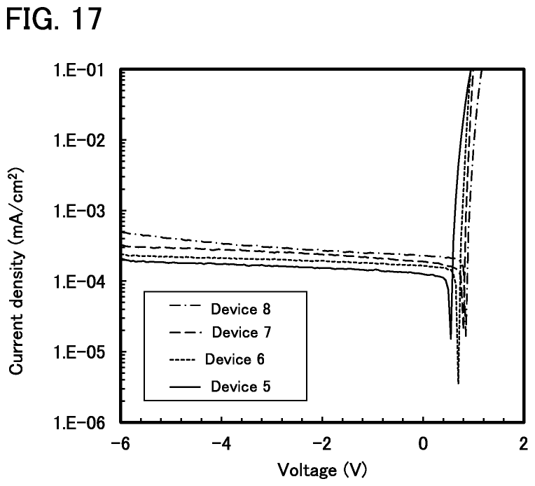

FIG. 17 is a graph showing voltage-current density characteristics of the device 5, the device 6, the device 7, and the device 8 in a state in a state where light irradiation was performed.

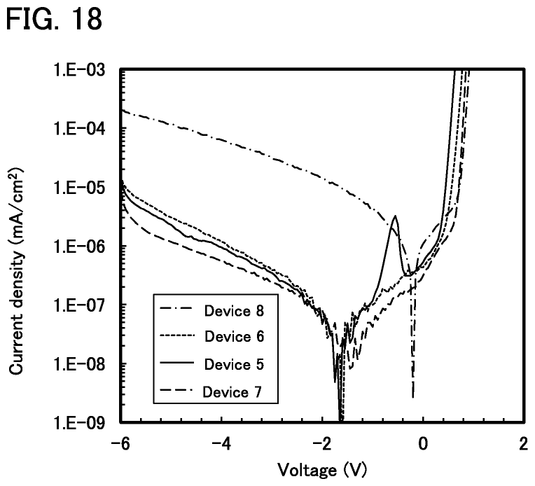

FIG. 18 is a graph showing voltage-current density characteristics of the device 5, the device 6, the device 7, and the device 8 in a state where light irradiation was not performed.

### <Device 5>

The fabricated device 5, which is described in this example, has a structure similar to that of the photoelectric conversion device 550S (see FIG. 12B). The structure of the device 5 is different from that of the device 1 described in Example 1. Specifically, the device 5 is different from the device 1 in that the layer 114P and the layer 114N were provided instead of the layer 114S.

The photoelectric conversion device 550S includes the electrode 551S, the electrode 552S, the layer 112, a layer 114, and the layer 113. The layer 112 is held between the electrode 551S and the electrode 552S, the layer 114S is held between the electrode 552S and the layer 112, the layer 113 is held between the electrode 552S and the layer 114S, and the layer 113 has higher electron mobility than the layer 112.

### <<Structure of Device 5>>

Table 6 shows the structure of the device 5.

TABLE 6

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114N | EtHex-PTCDI | | 48 |
| Layer | 114P | DPAPhA | | 12 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 5>>

The device 5 described in this example was fabricated using a method including the following steps.

Note that the fabrication method of the device 5 is different from the fabrication method of the device 1 in that the layer 114P was formed instead of the layer 114S in the fifth step and that a step 5-2 is provided between the fifth step and the sixth step. Different portions are described in detail here, and the above description is referred to for portions formed by a similar method.

### [Fifth Step]

In the fifth step, the layer 114P was formed over the layer 112. Specifically, a material was evaporated by a resistance-heating method.

Note that the layer 114P contains DPAPhA and has a thickness of 12 nm.

### [Step 5-2]

In the 5-2 step, the layer 114N was formed over the layer 114P. Specifically, materials were co-evaporated by a resistance-heating method.

Note that the layer 114N contains EtHex-PTCDI and has a thickness of 48 nm.

### <<Operation Characteristics of Device 5>>

The operation characteristics of the device 5 were measured at room temperature (see FIG. 16 to FIG. 18).

Table 2 shows main initial characteristics of the fabricated device.

The device 5 was found to have favorable photoelectric conversion characteristics. For example, the device 5 had an EQE of greater than or equal to 0.6% and less than 7% with respect to light with a wavelength of greater than or equal to 425 nm and less than or equal to 600 nm (see FIG. 16). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 17 and FIG. 18).

### <Device 6>

The fabricated device 6, which is described in this example, has a structure similar to that of the photoelectric conversion device 550S (see FIG. 12B). The structure of the device 6 is different from that of the device 5 in the layer 114P. Specifically, the device 6 is different from the device 5 in that the layer 114P contains TTPA instead of DPAPhA.

### <<Structure of Device 6>>

Table 7 shows the structure of the device 6.

TABLE 7

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114N | EtHex-PTCDI | | 48 |
| Layer | 114P | TTPA | | 12 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 6>>

The device 6 described in this example was fabricated using a method including the following steps.

Note that the fabrication method of the device 6 is different from the fabrication method of the device 5 in that TPPA was deposited by evaporation instead of DPAPhA in the fifth step. Different portions are described in detail here, and the above description is referred to for portions formed by a similar method.

### [Fifth Step]

In the fifth step, the layer 114P was formed over the layer 112. Specifically, the material was evaporated by a resistance-heating method.

Note that the layer 114P contains TPPA and has a thickness of 12 nm.

### <<Operation Characteristics of Device 6>>

The operation characteristics of the device 6 were measured at room temperature (see FIG. 16 to FIG. 18).

Table 2 shows main initial characteristics of the fabricated device.

The device 6 was found to have favorable photoelectric conversion characteristics. For example, the device 6 had an EQE of greater than or equal to 0.47% and less than 12% with respect to light with a wavelength of greater than or equal to 425 nm and less than or equal to 600 nm (see FIG. 16). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 17 and FIG. 18).

### <Device 7>

The fabricated device 7, which is described in this example, has a structure similar to that of the photoelectric conversion device 550S (see FIG. 12B). The structure of the device 7 is different from that of the device 5 in the layer 114P. Specifically, the device 7 is different from the device 5 in that the layer 114P contains 2Ph-mmtBuDPhA2Anth instead of DPAPhA.

### <<Structure of Device 7>>

Table 108 shows the structure of the device 7.

TABLE 8

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114N | EtHex-PTCDI | | 48 |
| Layer | 114P | 2Ph-mmAdtBuDPhA2Anth-02 | | 12 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 7>>

The device 7 described in this example was fabricated using a method including the following steps.

Note that the fabrication method of the device 7 is different from the fabrication method of the device 5 in that 2Ph-mmtBuDPhA2Anth was deposited by evaporation instead of DPAPhA in the fifth step. Different portions are described in detail here, and the above description is referred to for portions formed by a similar method.

### [Fifth Step]

In the fifth step, the layer 114P was formed over the layer 112. Specifically, the material was evaporated by a resistance-heating method.

Note that the layer 114P contains 2Ph-mmtBuDPhA2Anth and has a thickness of 12 nm.

### <<Operation Characteristics of Device 7>>

The operation characteristics of the device 7 were measured at room temperature (see FIG. 16 to FIG. 18).

Table 102 shows main initial characteristics of the fabricated device.

The device 7 was found to have favorable photoelectric conversion characteristics. For example, the device 7 had an EQE of greater than or equal to 0.5% and less than 8% with respect to light with a wavelength of greater than or equal to 425 nm and less than or equal to 600 nm (see FIG. 16). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 17 and FIG. 18).

### <Device 8>

The fabricated device 8, which is described in this example, has a structure similar to that of the photoelectric conversion device 550S (see FIG. 12B). The structure of the device 8 is different from that of the device 5 in the layer 114P. Specifically, the device 8 is different from the device 5 in that the layer 114P contains 2Ph-mmAdtBuDPhA2Anth-02 instead of DPAPhA.

### <<Structure of Device 8>>

Table 9 shows the structure of the device 8.

TABLE 9

| | Reference | | Composition | |
|---|---|---|---|---|
| Component | numeral | Material | ratio | Thickness/nm |
| Layer | CAP | DBT3P-II | | 80 |
| Electrode | 552S | Ag:Mg | 3:0.3 | 10 |
| Layer | 105 | LiF | | 1 |
| Layer | 113A | NBPhen | | 10 |
| Layer | 113B | 2mDBTBPDBq-II | | 10 |
| Layer | 114N | EtHex-PTCDI | | 48 |
| Layer | 114P | 2Ph-mmAdtBuDPhA2Anth-02 | | 12 |
| Layer | 112 | BBABnf | | 40 |
| Layer | 104 | BBABnf:OCHD-003 | 1:0.1 | 11 |
| Electrode | 551S | ITSO | | 100 |
| Reflective film | REF | APC | | 100 |

### <<Fabrication Method of Device 8>>

The device 8 described in this example was fabricated using a method including the following steps.

Note that the fabrication method of the device 8 is different from the fabrication method of the device 5 in that 2Ph-mmAdtBuDPhA2Anth-02 was deposited by evaporation instead of DPAPhA in the fifth step. Different portions are described in detail here, and the above description is referred to for portions formed by a similar method.

### [Fifth Step]

In the fifth step, the layer 114P was formed over the layer 112. Specifically, the material was evaporated by a resistance-heating method.

Note that the layer 114P contains 2Ph-mmAdtBuDPhA2Anth-02 and has a thickness of 12 nm.

### <<Operation Characteristics of Device 8>>

The operation characteristics of the device 8 were measured at room temperature (see FIG. 16 to FIG. 18).

Table 2 shows main initial characteristics of the fabricated device.

The device 8 was found to have favorable photoelectric conversion characteristics. For example, the device 8 had an EQE of greater than or equal to 0.4% and less than 11% with respect to light with a wavelength of greater than or equal to 425 nm and less than or equal to 600 nm (see FIG. 16). When the electrode 551S was supplied with a potential in the range of −6 V to 0 V inclusive relative to the potential of the electrode 522S, the density of current flowing in a state where light irradiation was performed was sufficiently higher than the density of current flowing in a state where light irradiation was not performed (see FIG. 17 and FIG. 18).

### REFERENCE NUMERALS

ANO: conductive film, AMP1: amplifier circuit, AMP2: amplifier circuit, CF: coloring layer, C21: capacitor, C22: capacitor, C31: capacitor, CAPSEL: conductive film, CDSVDD: conductive film, CDSVSS: conductive film, CDSBIAS: conductive film, CL: conductive film, FD: node, G1: conductive film, G2: conductive film, IN1: terminal, IN2: terminal, IN3: terminal, M21: transistor, M31: transistor, M32: transistor, N21: node, N22: node, OUT: terminal, RS: conductive film, S1: conductive film, S1g: conductive film, S2: conductive film, SE: conductive film, SW21: switch, SW22: switch, SW23: switch, SW31: switch, SW32: switch, SW33: switch, TX: conductive film, V0: conductive film, VCOM2: conductive film, VCL: conductive film, VCP: conductive film, VIV: conductive film, VLEN: conductive film, VPD: conductive film, VPI: conductive film, VR: conductive film, WX: conductive film, 103S: unit, 103X: unit, 104: layer, 105: layer, 111X: layer, 112: layer, 113: layer, 114N: layer, 114P: layer, 114S: layer, 231: region, 520: functional layer, 521: insulating film, 528: insulating film, 530S: pixel circuit, 530X: pixel circuit, 540: functional layer, 550S: photoelectric conversion device, 550X: light-emitting device, 551S: electrode, 551X: electrode, 551XS: gap, 552S: electrode, 552X: electrode, 610: package substrate, 611: package substrate, 620: cover glass, 621: lens cover, 630: adhesive, 635: lens, 640: bump, 641: land, 650: image sensor chip, 651: image sensor chip, 660: electrode pad, 661: electrode pad, 670: wire, 671: wire, 690: IC chip, 700: device, 702S: pixel, 702X: pixel, 703: pixel, 770: functional layer, 911: housing, 912: display portion, 913: speaker, 919: camera, 932: display portion, 933: housing and wristband, 939: camera, 951: support base, 952: camera unit, 953: protection cover, 961: housing, 962: shutter button, 963: microphone, 965: lens, 967: light-emitting portion, 971: housing, 972: housing, 973: display portion, 974: operation key, 975: lens, 976: connection portion, 977: speaker, 978: microphone, 981: housing, 982: display portion, 983: operation button, 984: external connection port, 985: speaker, 986: microphone, 987: camera

1-8. (canceled)

9. A material for a photoelectric conversion device, the photoelectric conversion device comprising a first electrode, a second electrode, a first layer, a second layer and a third layer,
wherein the first layer is between the first electrode and the second electrode,
wherein the second layer is between the first layer and the second electrode,
wherein the third layer is between the second layer and the second electrode,
wherein the third layer has higher electron mobility than the first layer,
wherein the material is used in the second layer,
wherein the material comprises an anthracene skeleton, and
wherein the anthracene skeleton is substituted with any one of a diarylamino group, a diheteroarylamino group and an arylheteroarylamino group.

10. The material for a photoelectric conversion device according to claim 9, wherein 9-position of the anthracene skeleton is substituted with any one of a diarylamino group, a diheteroarylamino group and an arylheteroarylamino group.

11. The material for a photoelectric conversion device according to claim 10,
wherein 9-position of the anthracene skeleton is substituted with an diarylamino group,
wherein the anthracene skeleton further has a substituent at either or both of 2-position and 6-position of the anthracene skeleton,
wherein the substituent is any of a substituted or unsubstituted aryl group having 6 to 25 carbon atoms, a substituted or unsubstituted heteroaryl group having 2 to 25 carbon atoms, and a substituted or unsubstituted diarylamino group, and
wherein an aryl group of the substituted or unsubstituted diarylamino group is a substituted or unsubstituted aryl group having 6 to 25 carbon atoms or a substituted or unsubstituted heteroaryl group having 2 to 25 carbon atoms.

12. The material for a photoelectric conversion device according to claim 10,
wherein said any one of a diarylamino group, a diheteroarylamino group and an arylheteroarylamino group comprises a first ring and a second ring each bonded to a nitrogen atom, and
wherein the first ring and the second ring are same.

13. The material for a photoelectric conversion device according to claim 10,
wherein said any one of a diarylamino group, a diheteroarylamino group and an arylheteroarylamino group comprises a first ring and a second ring each bonded to a nitrogen atom, and
wherein the first ring and the second ring are different from each other.

14. The material for a photoelectric conversion device according to claim 10,
wherein said any one of a diarylamino group, a diheteroarylamino group and an arylheteroarylamino group comprises a first ring and a second ring each bonded to a nitrogen atom, and
wherein the first ring and the second ring form a ring to bond to each other.

15. The material for a photoelectric conversion device,
wherein the material is represented by Formula (G1): 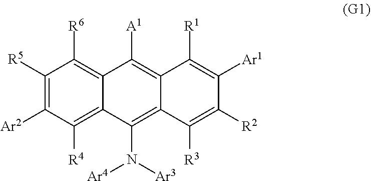
wherein each of A¹, Ar¹ and Ar² independently represents any one of hydrogen or deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, and a substituted or unsubstituted diarylamino group,
wherein two aryl groups of the substituted or unsubstituted diarylamino group in each of A¹, Ar¹ and Ar² are each a substituted or unsubstituted aryl group having 6 to 25 carbon atoms.
wherein each of R¹ to R⁶ independently represents any one of hydrogen or deuterium, a substituted or unsubstituted alkyl group having 1 to 13 carbon atoms, a substituted or unsubstituted cycloalkyl group having 3 to 10 carbon atoms, a substituted or unsubstituted aryl group having 6 to 25 carbon atoms, a substituted or unsubstituted heteroaryl group having 2 to 25 carbon atoms, and a substituted or unsubstituted alkoxy group having 1 to 13 carbon atoms, and
wherein each of Ar³ and Ar⁴ independently represents one of a substituted or unsubstituted aryl group having 6 to 25 carbon atoms and a substituted or unsubstituted heteroaryl group having 2 to 25 carbon atoms.

16. A display device comprising a pixel set,
wherein the pixel set comprises a first pixel and a second pixel,
wherein the first pixel comprises a light-emitting device,
wherein the second pixel comprises a photoelectric conversion device,
wherein the photoelectric conversion device is adjacent to the light-emitting device, and
wherein the photoelectric conversion device comprises the material according to claim 9.

17. The display device according to claim 16, further comprising:
a first functional layer comprising the photoelectric conversion device and the light-emitting device; and
a second functional layer comprising:
a first pixel circuit electrically connected to the light-emitting device; and
a second pixel circuit electrically connected to the photoelectric conversion device,
wherein the first functional layer and the second functional layer overlap each other,
wherein the first pixel circuit is included in the first pixel, and
wherein the second pixel circuit is included in the second pixel.

18. The display device according to claim 16,
wherein the light-emitting device comprises a third electrode, a fourth electrode, and a unit between the third electrode and the fourth electrode,
wherein the unit comprises:
the first layer between the third electrode and the fourth electrode;
the third layer between the first layer and the fourth electrode; and
a fourth layer between the third layer and the first layer,
wherein the third layer has higher electron mobility than the first layer, and
wherein the fourth layer comprises a light-emitting material.

19. A photoelectric conversion device comprising:
a first electrode;
a photoelectric conversion layer over the first electrode, the photoelectric conversion layer comprising a first material; and
a second electrode over the photoelectric conversion layer,
wherein the first material has an anthracene skeleton,
wherein the anthracene skeleton is substituted with an amino group at 9-position,
wherein the amino group is substituted with a first substituent and a second substituent, and
wherein each of the first substituent and the second substituent is one of a substituted or unsubstituted aryl group having 6 to 25 carbon atoms and a substituted or unsubstituted heteroaryl group having 2 to 25 carbon atoms.

20. The photoelectric conversion device according to claim 19, wherein the photoelectric conversion layer further comprises an electron-accepting material.
